(19) United States
(12) Patent Application Publication
(10) Pub. No.: US 2024/0400596 A1
(43) Pub. Date: Dec. 5, 2024

(54) PLATINUM-BASED PHOTOINITIATOR AND PHOTOCROSSLINKER, THEIR USE FOR ADDUCT FORMATION

(71) Applicant: City University of Hong Kong, Kowloon (HK)

(72) Inventors: Guangyu Zhu, Kowloon (HK); Jiaqian Xu, Kowloon (HK)

(21) Appl. No.: 18/326,243

(22) Filed: May 31, 2023

(51) Int. Cl. C07F 15/00; A61K 9/06; A61P 31/04; C08J 3/075; C08J 3/28; C08J 7/12; C08K 5/00

(52) U.S. Cl. CPC C07F 15/0093; A61K 9/06; A61P 31/04; C08J 3/075; C08J 3/28; C08J 7/123; C08K 5/0091; C08J 2333/26

(57) ABSTRACT

A platinum-based adduct including a substrate to which a photolyzed active platinum species is attached, of the photolyzed platinum active species is generated from a platinum(IV) complex having a structure of Formula (Ia. A method of preparing the platinum-based adduct. An antibacterial platinum-based adduct. Uses of the platinum(IV) complex as a photoinitiator for hydrogelation, and as a photocrosslinker for hydrogelation or protein labeling are also addressed.

### TECHNICAL FIELD

The present invention relates to a platinum-based adduct for example particularly, but not exclusively, a platinum-hydrogel or a platinum-biomolecule adduct comprising a hydrogel or a biomolecule to which a photolyzed active species of a platinum(IV) complex is attached and a method for preparing the same.

### BACKGROUND OF THE INVENTION

Biomaterials play an important role in biomedical applications. In particular, polymeric materials such as hydrogel-based materials and protein-based materials have been attracting much attention and being used in a wide range of biomedical applications such as skin scaffolds, drug delivery, medical imaging, tissue engineering, etc. However, it is believed that typical fabrication for the above materials, such as those fabrication involving photoresponsive compounds/molecules may suffer from drawbacks such as complicated fabrication procedures, low crosslinking and/or labeling efficiency, etc.

In one example, in considering the fabrication of antibacterial hydrogel such as an antibacterial gelatin, it may require separate fabrication steps including initial functionalization of gelatin with methacrylamide, followed by photocrosslinking the modified peptides with a photoinitiator, along with or subsequently along with the indrocution of antibacterial agents. However, it is believed that antibacterial agents such as inorganic antibacterial agents, particularly those taking the form of nanoparticles, may lack stability and dispersion in the hydrogel, which further enhances the fabrication difficulty. Thus, it is appreciated that a simple, facile approach for fabricating as such would be barely achieved.

In another example, it is appreciated that many of the photoreactive protein labeling agents may involve carbine-enabled C—H and N—H insertion chemistry, which may suffer from low crosslinking efficiency as a result of the short-ranged species.

Thus, there remains a strong need in providing a new or otherwise improved photoresponsive compound such as a photoinitiator and/or photocrosslinker for biomaterial fabrication in biomedical applications.

### SUMMARY OF THE INVENTION

In a first aspect of the present invention, there is provided a platinum-based adduct comprising a substrate to which a photolyzed active platinum species is attached, the photolyzed active platinum species is generated from a platinum(IV) complex having a structure of Formula (Ia):

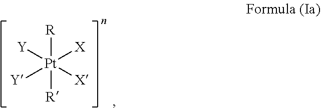

wherein X, X′, Y, Y′, R and R′ are electron donor ligands; and n is zero, any positive charge or negative charge. Optionally, at least one of the electron donor ligand comprises one or more of nitrogen-containing ligands, oxygen-containing ligands, phosphorous-containing ligands, sulfur-containing ligands or halogen-containing ligands. Optionally, the photolyzed active platinum species comprises a platinum(I) radical.

In an optional embodiment, the platinum(IV) complex has a structure of Formula (Ib):

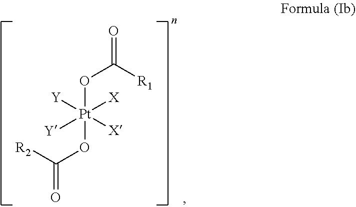

wherein:
X, X′, Y, and Y′ are independently selected from the group consisting of ammonia, halide, oxalate, diamines, dicarboxylate, and glycolate,
R₁ and R₂ are independently selected from the group consisting of a linear or branched C1-C4 alkyl chain, a carboxyl group, and a carboxylate ester-containing group; and
n is zero charge.

Optionally, X and X′ are linked to form a first bidentate ligand. Optionally, Y and Y′ are linked to form a second bidentate ligand. It is optional that the substrate is hydrogel-based or biomolecule-based.

In an optional embodiment, R₁ and R₂ are independently selected from the group consisting of —CH₃, —CH₂CH₃, —CH₂CH₂CH₃, —CH₂COOH, —CH₂CH₂COOH, —CH₂COOCH₃, —CH2CH2COOCH₃ and —CH₂COOCH₂CH₃.

In an optional embodiment, X and X′ are ammonia and/or chloride; the second bidentate ligand is a diamine; R₁ and R₂ are —CH₃, —CH₂CH₂COOH, or —CH₂CH₂COOCH₃; and n is zero charge.

In an optional embodiment, Y and Y′ are ammonia and/or chloride, the first bidentate ligand is cyclobutane dicarboxylate and/or oxalate, R₁ and R₂ are —CH₃, —CH₂CH₂COOH or —CH₂CH₂COOCH₃; and n is zero charge.

In an embodiment of the invention, the platinum(IV) complex has a structure of Formula (IIa), Formula (IIb), Formula (IIc), Formula (IId), Formula (IIe), or Formula (IIf):

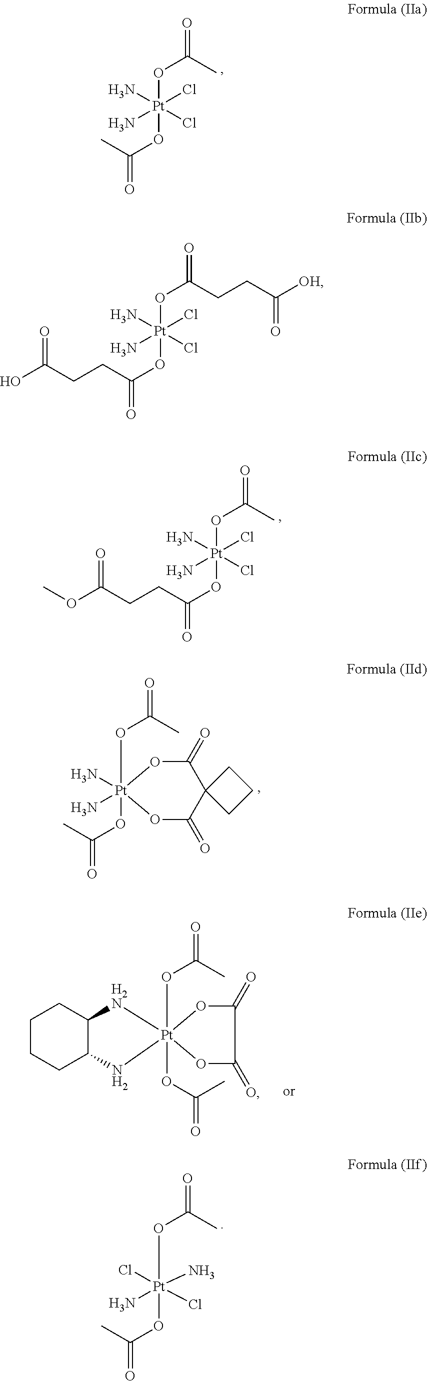

Optionally, the platinum(IV) complex of Formula (IIa) generates one or more of acetate radical, cisplatin, chlorine radical. ROS, and the photolzyed active platinum species.

In an optional embodiment, the chemical structure of the platinum(IV) complex remains unchanged for at least 30 min in the absence of radiation.

Optionally, the hydrogel-based substrate comprises any one of polyacrylamide or gelatin. In an embodiment of the invention, the hydrogel-based substrate comprises any one of 10% acrylamide/bis-acrylamide (29:1) and 5% gelatin.

Optionally, the biomolecule-based substrate comprises any one of an amino acid or polypeptide. In an embodiment of the invention, the amino acid is selected from cysteine, methionine, tryptophan, and tyrosine, and the polypeptide is BSA.

It is optional that the platinum(IV) complex further comprises a functional moiety that is attached to the platinum(IV) complex for functionalizing the platinum-based adduct. Optionally, the platinum(IV) complex has a structure of Formula (IIIa):

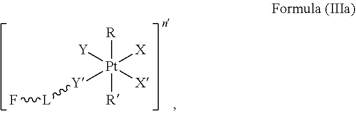

wherein:
X, X′, Y, Y′, R and R′ are as defined above;
L is a linker group;
F is the functional moiety for functionalizing the platinum-based adduct; and
n′ is zero, any positive charge or negative charge.

It is optional that the functional moiety has a reactive group that is complementary to the reactive group of a fluorescent reporting unit. In an optional embodiment, the platinum(IV) complex has a structure of Formula (IIIb):

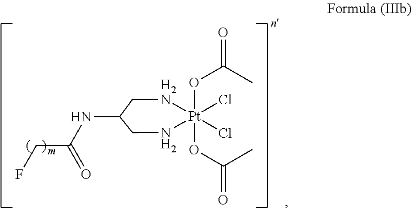

wherein:
m is 1-6;
F is the functional moiety which is a biorthogonal group selected from the group consisting of azide, terminal alkyne, activated cyclooctyne, and tetrazine; and
n′ is zero charge.

In an embodiment of the invention, the platinum(IV) has a structure of Formula (IV):

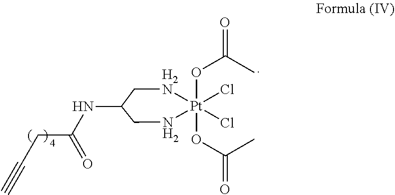

In a second aspect of the present invention, there is provided a photoinitiator for hydrogelation comprising a photolyzed active platinum species generated from a platinum(IV) complex having a structure of Formula (Ia):

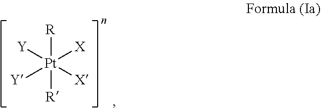

wherein X, X′, Y, Y′, R and R′ are electron donor ligands; and n is zero, any positive charge or negative charge.

In an optional embodiment, the platinum(IV) complex has a structure of Formula (Ib):

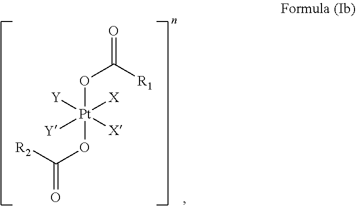

wherein:
X, X′, Y, and Y′ are independently selected from the group consisting of ammonia, halide, oxalate, diamines, dicarboxylate, and glycolate,
R₁ and R₂ are independently selected from the group consisting of a linear or branched C1-C4 alkyl chain, a carboxyl group, and a carboxylate ester-containing group; and
n is zero charge.

It is optional that 75% of photolyzed platinum species is retained after exposure to radiation.

In an embodiment of the invention, the platinum(IV) complex has a structure of Formula (IIa):

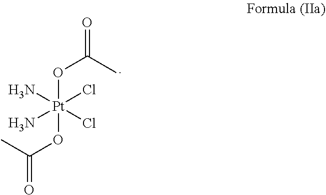

In a third aspect of the present invention, there is provided a photocrosslinker for hydrogelation or protein labeling comprising a photolyzed active platinum species generated from a platinum(IV) complex having a structure of Formula (Ia) or Formula (IIIa):

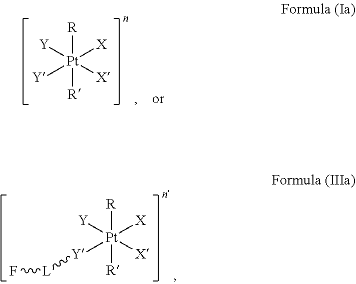

wherein:
X, X, Y, Y, R and R′ are electron donor ligands;
L is a linker group;
F is a functional moiety for protein labeling; and
n and n′ are each independently being zero, any positive charge or negative charge.

In an optional embodiment, the platinum(IV) complex has a structure of Formula (Ib):

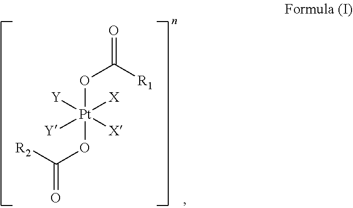

wherein:
X, X′, Y, and Y′ are independently selected from the group consisting of ammonia, halide, oxalate, diamines, dicarboxylate, and glycolate,
R₁ and R₂ are independently selected from the group consisting of a linear or branched C1-C4 alkyl chain, a carboxyl group, and a carboxylate ester-containing group; and
n is zero charge.

In an embodiment of the invention, the platinum(IV) complex has a structure of Formula (IIa):

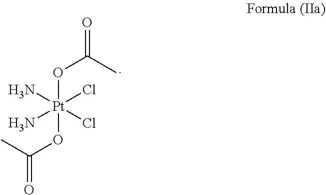

In an optional embodiment, the platinum(IV) complex has a structure of Formula (IIIb):

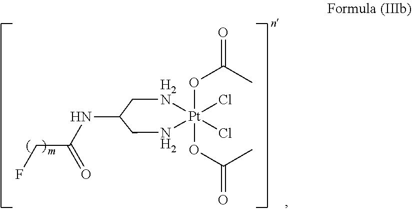

wherein:
m is 1-6;
F is the functional moiety which is a biorthogonal group selected from the group consisting of azide, terminal alkyne, activated cyclooctyne, and tetrazine; and
n′ is zero charge.

In an embodiment of the invention, the platinum(IV) has a structure of Formula (IV):

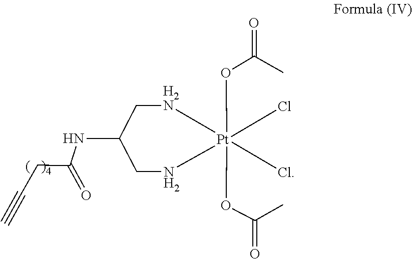

In a fourth aspect of the present invention, there is provided an antibacterial platinum-based adduct comprising a substrate to which a photolyzed active platinum species is attached, the photolyzed active platinum species is generated from a platinum(IV) complex having a structure of Formula (IIa):

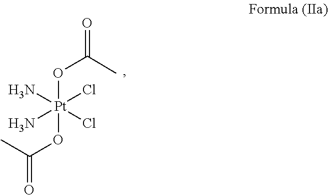

Optionally, the substrate comprises polyacrylamide and the adduct has an antibacterial activity against E. coli (DH5α).

In the fifth aspect of the present invention, there is provided a method of preparing the platinum-based adduct in accordance with the first aspect, comprising the steps of: providing a solution mixture comprising a hydrogel precursor or a biomolecule, and a platinum(IV) complex having a structure of Formula (Ia); and administering to the solution mixture radiation in an amount and of a wavelength effective to activate the platinum(IV) complex to form a photolyzed active platinum species comprising a platinum(I) radical, which reacts with the hydrogel precursor or the biomolecule to form the platinum-based adduct.

Optionally, the radiation is UV radiation. It is optional that the UV radiation has a wavelength of about 360 nm to about 370 nm. It is also optional that the UV radiation is applied at a power density from about 9 mW/cm² to about 10 mW/cm².

### BRIEF DESCRIPTION OF DRAWINGS

The invention will now be more particularly described, by way of example only, with reference to the accompanying drawings, in which:

FIG. 1A is a ¹H NMR spectrum of cisPt(IV)-1 (i.e., platinum(IV) complex of Formula (IIa)) in DMSO-d₆;

FIG. 1B is a ¹³C NMR spectrum of cisPt(IV)-1 in DMSO-d₆;

FIG. 1C is a ¹⁹⁵Pt NMR spectrum of cisPt(IV)-1 in DMSO-d₆;

FIG. 1D is an ESI-MS spectrum of cisPt(IV)-1;

FIG. 1E is a HPLC chromatogram of cisPt(IV)-1;

FIG. 2A is a ¹H NMR spectrum of cisPt(IV)-2 (i.e., platinum(IV) complex of Formula (IIb)) in DMSO-d₆;

FIG. 2B is a ¹³C NMR spectrum of cisPt(IV)-2 in DMSO-d₆;

FIG. 2C is a ¹⁹⁵Pt NMR spectrum of cisPt(IV)-2 in DMSO-d₆;

FIG. 2D is an ESI-MS spectrum of cisPt(IV)-2;

FIG. 2E is a HPLC chromatogram of cisPt(IV)-2;

FIG. 3A is a ¹H NMR spectrum of cisPt(IV)-3 (i.e., platinum(IV) complex of Formula (IIc)) in DMSO-d₆;

FIG. 3B is a ¹³C NMR spectrum of cisPt(IV)-3 in DMSO-d₆;

FIG. 3C is a ¹⁹⁵Pt NMR spectrum of cisPt(IV)-3 in DMSO-d₆;

FIG. 3D is an ESI-MS spectrum of cisPt(IV)-3;

FIG. 3E is a HPLC chromatogram of cisPt(IV)-3;

FIG. 4A is a ¹H NMR spectrum of carPt(IV) (i.e., platinum(IV) complex of Formula (IId)) in DMSO-d₆;

FIG. 4B is a ¹³C NMR spectrum of carPt(IV) in DMSO-d₆;

FIG. 4C is a ¹⁹⁵Pt NMR spectrum of carPt(IV) in DMSO-d₆;

FIG. 4D is an ESI-MS spectrum of carPt(IV);

FIG. 4E is a HPLC chromatogram of carPt(IV);

FIG. 5A is a ¹H NMR spectrum of oxaPt(IV) (i.e., platinum(IV) complex of Formula (IIe)) in DMSO-d₆;

FIG. 5B is a ¹³C NMR spectrum of oxaPt(IV) in DMSO-d₆;

FIG. 5C is a ¹⁹⁵Pt NMR spectrum of oxaPt(IV) in DMSO-d₆;

FIG. 5D is an ESI-MS spectrum of oxaPt(IV);

FIG. 5E is a HPLC chromatogram of oxaPt(IV);

FIG. 6A is a ¹H NMR spectrum of transPt(IV) (i.e., platinum(IV) complex of Formula (IIf)) in DMSO-d₆;

FIG. 6B is a ¹³C NMR spectrum of transPt(IV) in DMSO-d₆;

FIG. 6C is a ¹⁹⁵Pt NMR spectrum of transPt(IV) in DMSO-d₆;

FIG. 6D is an ESI-MS spectrum of transPt(IV);

FIG. 6E is a HPLC chromatogram of transPt(IV);

FIG. 7A is a ¹H NMR spectrum of alkyne-Pt(IV) (i.e., platinum(IV) complex of Formula (IV)) in DMSO-d₆;

FIG. 7B is a ¹³C NMR spectrum of alkyne-Pt(IV) in DMSO-d₆.

FIG. 7C is a ¹⁹⁵Pt NMR spectrum of alkyne-Pt(IV) in DMSO-d₆;

FIG. 7D is an ESI-MS spectrum of alkyne-Pt(IV);

FIG. 7E is a HPLC chromatogram of alkyne-Pt(IV);

FIG. 8A is a ¹H NMR spectrum of PC-1 in chloroform-d;

FIG. 8B is a ¹³C NMR spectrum of PC-1 in chloroform-d;

FIG. 8C is an ESI-MS spectrum of PC-1;

FIG. 8D is a HPLC chromatogram of PC-1;

FIG. 9A is a ¹H NMR spectrum of PC-2 in chloroform-d;

FIG. 9B is a ¹³C NMR spectrum of PC-2 in chloroform-d;

FIG. 9C is an ESI-MS spectrum of PC-2;

FIG. 9D is a HPLC chromatogram of PC-2;

FIG. 10A is a ¹H NMR spectrum of alkyne-Pt(II) in DMF-d₇;

FIG. 10B is a ¹³C NMR spectrum of alkyne-Pt(II) in DMF-d₇;

FIG. 10C is a ¹⁹⁵Pt NMR spectrum of alkyne-Pt(II) in DMF-d₇;

FIG. 10D is an ESI-MS spectrum of alkyne-Pt(II);

FIG. 11 is a schematic diagram showing the chemical structures of the Pt(II) and Pt(IV) complexes disclosed in the present disclosure;

FIG. 12 shows a LED light source (365 nm) used in the present disclosure.

FIG. 13A shows the RP-HPLC (254 nm) chromatograms of cisPt(IV)-1 in water with or without irradiation (365 nm, 9.8 mW/cm²) for 10 min;

FIG. 13B is an ESI-HRMS spectrum of cisPt(IV)-1 showing cisplatin as the photolyzed product of cisPt(IV)-1;

FIG. 14 shows the photolyzed product of cisPt(IV)-2 in water upon UV (365 nm) irradiation as characterized by HPLC (254 nm) and ESI-MS;

FIG. 15 shows the photolyzed product of cisPt(IV)-3 in water upon UV (365 nm) irradiation as characterized by HPLC (254 nm) and ESI-MS;

FIG. 16 shows the photolyzed product of carPt(IV) in water upon UV (365 nm) irradiation as characterized by HPLC (254 nm) and ESI-MS;

FIG. 17 shows the photolyzed product of oxaPt(IV) in water upon UV (365 nm) irradiation as characterized by HPLC (254 nm) and ESI-MS;

FIG. 18 shows the photolyzed product of transPt(IV) in water upon UV (365 nm) irradiation as characterized by HPLC (254 nm);

FIG. 19 shows the percentage of intact Pt(II) and Pt(IV) complexes upon irradiation (365 nm, 9.8 mW/cm²) for 10 mins;

FIG. 20A shows the photolysis of cisplatin in water by UV (365 nm) irradiation, monitored by HPLC (254 nm);

FIG. 20B shows the photolysis of oxaliplatin in water by UV (365 nm) irradiation, monitored by HPLC (254 nm);

FIG. 20C shows the photolysis of carboplatin in water by UV (365 nm) irradiation, monitored by HPLC (254 nm);

FIG. 20D shows the photolysis of transplatin in water by UV (365 nm) irradiation, monitored by HPLC (254 nm);

FIG. 21A shows the absorption spectra of methylene blue aqueous solution with or without UV irradiation (365 nm, 9.8 mW/cm²);

FIG. 21B shows the absorption spectra of methylene blue aqueous solution containing cisPt(IV)-1 with or without UV irradiation (365 nm, 9.8 mW/cm²);

FIG. 21C shows the absorption spectra of methylene blue aqueous solution containing cisPt(IV)-2 with or without UV irradiation (365 nm, 9.8 mW/cm²);

FIG. 21D shows the absorption spectra of methylene blue aqueous solution containing cisPt(IV)-3 with or without UV irradiation (365 nm, 9.8 mW/cm²);

FIG. 21E shows the absorption spectra of methylene blue aqueous solution containing cisplatin with or without UV irradiation (365 nm, 9.8 mW/cm²);

FIG. 22A shows the absorption spectra of methylene blue aqueous solution with or without UV irradiation (365 nm, 9.8 mW/cm²);

FIG. 22B shows the absorption spectra of methylene blue aqueous solution containing carPt(IV) with or without UV irradiation (365 nm, 9.8 mW/cm²);

FIG. 22C shows the absorption spectra of methylene blue aqueous solution containing carboplatin with or without UV irradiation (365 nm, 9.8 mW/cm²);

FIG. 23A shows the absorption spectra of methylene blue aqueous solution with or without UV irradiation (365 nm, 9.8 mW/cm²);

FIG. 23B shows the absorption spectra of methylene blue aqueous solution containing oxaPt(IV) with or without UV irradiation (365 nm, 9.8 mW/cm²);

FIG. 23C shows the absorption spectra of methylene blue aqueous solution containing oxaliplatin with or without UV irradiation (365 nm, 9.8 mW/cm²);

FIG. 24A shows the absorption spectra of methylene blue aqueous solution with or without UV irradiation (365 nm, 9.8 mW/cm²);

FIG. 24B shows the absorption spectra of methylene blue aqueous solution containing transPt(IV) with or without UV irradiation (365 nm, 9.8 mW/cm²);

FIG. 24C shows the absorption spectra of methylene blue aqueous solution containing transplatin with or without UV irradiation (365 nm, 9.8 mW/cm²);

FIG. 25A shows the RP-HPLC (254 nm) chromatograms of cisPt(IV)-1 upon irradiation (365 nm, 9.8 mW/cm²) with or without DMPO. A new peak at 8.9 min was characterized as the acetate radical spin adducts DMPO-Ac;

FIG. 25B shows the RP-HPLC (254 nm) chromatograms of cisPt(IV)-1 upon irradiation (365 nm, 9.8 mW/cm²) with or without TEMPO;

FIG. 25C shows the RP-HPLC (254 nm) chromatograms of DMPO with or without irradiation (365 nm, 9.8 mW/cm²);

FIG. 25D shows the RP-HPLC (254 nm) chromatograms of TEMPO with or without irradiation (365 nm, 9.8 mW/cm²);

FIG. 26A shows the photostability of cisPt(IV)-1 in the presence of TEMPO in the dark;

FIG. 26B shows the photostability of cisPt(IV)-1 in the presence of DMPO in the dark;

FIG. 26C shows the photostability of cisPt(IV)-1 in the dark;

FIG. 27 shows photolysis of cisplatin with or without acetic acid in the presence of DMPO, monitored by HPLC (254 nm). The acetate radical-DMPO adduct (DMPO-Ac) was characterized by ¹H NMR as shown in the inserted ¹H NMR spectrum;

FIG. 28 is a schematic diagram illustrating the proposed mechanism of the spin-trapping of acetate radical with DMPO during the photolysis of cisPt(IV)-1. Acetate radical was formed during the photolysis of cisPt(IV)-1. In the presence of DMPO, the DMPO spin-adduct was formed, a nitroxide, and subsequently converted to its stable end product, nitrone;

FIG. 29A shows the platinum radical spin-adduct with TEMPO in the photolysis of cisPt(IV)-1, namely [Pt(NH₃)₂(TEMPO)Cl], characterized by ESI-HRMS (positive mode). Control experiments were also carried out with cisplatin;

FIG. 29B is a schematic diagram illustrating the proposed mechanism of the spin-trapping of platinum(I) radical with TEMPO during the photolysis of cisPt(IV)-1;

FIG. 29C shows the platinum radical spin-adduct with TEMPO in the photolysis of cisPt(IV)-1, namely [Pt(NH₃)₂(TEMPO)OH], characterized by ESI-HRMS (positive mode). Control experiments were also carried out with cisplatin;

FIG. 29D is a schematic diagram illustrating the proposed mechanism of the spin-trapping of platinum(I) radical with TEMPO during the photolysis of cisPt(IV)-1;

FIG. 30 shows an EPR spectrum of cisPt(IV)-1 in the presence of DMPO;

FIG. 31A shows UV/Vis absorption spectra of DPD (chlorine radical probe) in the photolysis of cisPt(IV)-1. Control experiments were carried out with cisplatin;

FIG. 31B shows UV/Vis absorption spectra of TMB (ROS, hydroxyl radical probe) in the photolysis of cisPt(IV)-1. Control experiments were carried out with cisplatin;

FIG. 31C shows UV/Vis absorption spectra of OPD (ROS, hydroxyl radical probe) in the photolysis of cisPt(IV)-1. Control experiments were carried out with cisplatin;

FIG. 32 shows the EPR spectrum of cisPt(IV)-1 in water containing TEMP upon irradiation, and the schematic diagram illustrating the proposed mechanism of the radical quenching with TEMPO during the photolysis of cisPt(IV)-1;

FIG. 33 shows the fluorescence spectra of DCFH for monitoring the ROS generated in the photolysis of cisPt(IV)-1;

FIG. 34A shows the fluorescence spectra of the DCFH solution containing cisPt(IV)-1 upon irradiation under normoxia and hypoxia conditions;

FIG. 34B shows the extracted ion intensity (cisplatin: 300.9608) chromatogram in the solution of cisPt(IV)-1 upon irradiation monitored by LC-HRMS. Unknown photolysis products that possess similar m/z as cisplatin and with the isotope distribution pattern containing platinum and chloride element were observed exclusively under normoxia condition;

FIG. 35A is a photograph showing the yellow precipitate visually observed in the saturated cisPt(IV)-1 solution containing 1 M DMPO.

FIG. 35B is a ESI-HRMS spectrum of the yellow precipitate in FIG. 35A, characterizing that the yellow precipitate as cisplatin;

FIG. 35C is a HPLC chromatogram of the s yellow precipitate in FIG. 35A, characterizing that the yellow precipitate as cisplatin;

FIG. 35D is a ¹H NMR spectrum of the yellow precipitate in FIG. 35A, characterizing that the yellow precipitate as cisplatin;

FIG. 36 shows the yield of cisplatin in the photolysis of cisPt(IV)-1 in the presence of different concentrations of DMPO, monitored by LC-HRMS;

FIG. 37A shows the LC-HRMS chromatogram of different concentration of cisplatin dissolved in water;

FIG. 37B is a plot of peak area against the concentration of cisplatin showing the standard curve of cisplatin. Each point corresponds to the major ion intensity of cisplatin [M+H]⁺: 300.9608 extracted from the LC-HRMS detection in FIG. 37A;

FIG. 38A shows the LC-HRMS chromatogram of cisPt(IV)-1 upon irradiation in the presence of DMPO. The major ion intensity of cisplatin [M+H]⁺: 300.9608 was extracted;

FIG. 38B shows the LC-HRMS chromatogram of cisPt(IV)-1 upon irradiation in the presence of TEMPO. The major ion intensity of cisplatin [M+H]⁺: 300.9608 was extracted;

FIG. 38C shows the yield of cisplatin in the photolysis of cisPt(IV)-1 containing different concentrations of TEMPO, as quantified by the standard curve;

FIG. 39 shows the percentage of the cisPt(IV)-1 remained with and without the presence of DMPO or TEMPO upon irradiation (365 nm, 9.8 mW/cm²);

FIG. 40A is a HPLC chromatogram (254 nm) of photolysis of cisPt(IV)-1 in water upon irradiation;

FIG. 40B is a HPLC chromatogram (254 nm) of photolysis of cisPt(IV)-1 in water containing DMPO upon irradiation;

FIG. 40C is a HPLC chromatogram (254 nm) of photolysis of cisPt(IV)-1 in water containing TEMPO upon irradiation;

FIG. 41 is a schematic diagram illustrating the proposed primary photolysis mechanism of cisPt(IV)-1;

FIG. 42 is a schematic diagram illustrating the photopolymerization of monomers with Pt(IV) complex as a photoinitiator;

FIG. 43 is a photograph showing the hydrogelation behavior of acrylamide containing cisPt(IV)-1 upon irradiation (365 nm, 9.8 mW/cm²);

FIG. 44 is a SEM image of Pt-gel;

FIG. 45A shows the electrophoresis of protein marker, BSA and A2780 cell lysate with the conventional APS/TEMED-Gel;

FIG. 45B shows the electrophoresis of protein marker, BSA and A2780 cell lysate with the Pt-gel;

FIG. 46 shows the release profile of platinum from the Pt-gel scaffold;

FIG. 47 shows photographs of E. coli on the LB agar plate treated with Pt-gel, 12959-gel, cisplatin, and PBS;

FIG. 48 shows the bacterial density of DH5α in LB liquid medium and Pt-gel coculture.

FIG. 49A shows positive ions for the adducts of cisPt(IV)-1 and cysteine upon irradiation, observed by ESI-HRMS and the corresponding assignments;

FIG. 49B shows positive ions for the adducts of cisPt(IV)-1 and methionine upon irradiation, observed by ESI-HRMS and the corresponding assignments;

FIG. 49C shows positive ions for the adducts of cisPt(IV)-1 and tryptophan upon irradiation, observed by ESI-HRMS and the corresponding assignments;

FIG. 49D shows positive ions for the adducts of cisPt(IV)-1 and tyrosine upon irradiation, observed by ESI-HRMS and the corresponding assignments;

FIG. 50 shows the percentage of platinum bound to BSA;

FIG. 51 is a schematic diagram illustrating the hydrogelation of gelatin;

FIG. 52 shows the photographs of Pt(IV)-Gelatin hydrogels at a gelatin concentration of 5% with or without irradiation (365 nm, 9.8 mW/cm²). Pt(II) as controls were carried out simultaneously;

FIG. 53A is a schematic diagram illustrating the design of the Pt(IV)-based photocrosslinker in accordance with an embodiment of the present invention;

FIG. 53B is a schematic diagram illustrating the chemical structures of the photocrosslinker disclosed in the present disclosure;

FIG. 54A shows the gel fluorescence (FL) and Coomassie blue (CBB) staining of photolabeled BSA by the Pt(IV)-based photocrosslinker;

FIG. 54B shows the quantification of the gel fluorescence intensity in FIG. 54A;

FIG. 55A shows the time-resolved BSA labeling by switching of the photostimuli;

FIG. 55B shows the gel fluorescence (FL) and Coomassie blue (CBB) staining of the pulse-chase labeling of BSA of FIG. 55A;

FIG. 56 shows the synthetic scheme 1 which summarizes the synthetic procedure of cisPt(IV)-1;

FIG. 57 shows the synthetic scheme 2 which summarizes the synthetic procedure of cisPt(IV)-2;

FIG. 58 shows the synthetic schemes 3A and 3B which respectively summarize the synthetic procedures of cisPt(IV)-3 and MeSuc-NHS;

FIG. 59 shows the synthetic scheme 4 which summarizes the synthetic procedure of carPt(IV);

FIG. 60 shows the synthetic scheme 5 which summarizes the synthetic procedure of oxaPt(IV);

FIG. 61 shows the synthetic scheme 6 which summarizes the synthetic procedure of transPt(IV);

FIG. 62 shows the synthetic scheme 7 which summarizes the synthetic procedure of alkyne-Pt(IV);

FIG. 63 shows the synthetic scheme 8 which summarizes the synthetic procedure of PC-1;

FIG. 64 shows the synthetic scheme 9 which summarizes the synthetic procedure of PC-2; and

FIG. 65 shows the synthetic scheme 10 which summarizes the synthetic procedure of alkyne-Pt(II).

### DETAILED DESCRIPTION OF OPTIONAL EMBODIMENT

As used herein, the forms “a”, “an”, and “the” are intended to include the singular and plural forms unless the context clearly indicates otherwise.

The words “example” or “exemplary” used in this invention are intended to serve as an example, instance, or illustration. Any aspect or design described in this disclosure as “exemplary” is not necessarily to be construed as preferred or advantageous over other aspects or designs. Rather, use of the words “example” or “exemplary” is intended to present concepts in a concrete fashion.

It is believed that much focus has been put on the exploitation of photolabile ligands to construct photoactivatable Pt(IV) complexes as a prodrug for chemotherapeutic applications. Without intending to be limited by theory, the inventors have, through their own research, trials, and experiments, devised that the Pt(IV) complexes may be used for biomedical applications, particularly by employing the Pt(IV) complexes with a Pt(II) anticancer drug core such as cisplatin, carboplatin, oxaliplatin and innocent axial ligands of, for example, acetic acid, succinic acid, and succinic ester and the like. It is found that the Pt(IV) complexes may be photolyzed readily upon photoexcitation, yielding various reactive radical species that enables the formation of biomaterials such as biochemical conjugates and/or biochemical adduct. Thus, it is appreciated that the Pt(IV) complexes may be used as a photoinitiator, photocrosslinker, and/or photoreactive labeling agent in various areas, including but not limited to chemical biology, medicinal chemistry, antineoplastic agents, etc.

According to the invention, there is provided a platinum-based adduct comprising a substrate to which a photolyzed active platinum species is attached, the photolyzed active platinum species is generated from a platinum(IV) complex (also known as Pt(IV) complex.

The term “substrate” as used herein denotes a molecule or a chemical compound which provides a surface or a three-dimensional (3D) space for which a reagent may be added thereto, such that a chemical product may be generated through a chemical reaction. For example, in this embodiment, the Pt(IV) complex may generate a photolyzed active platinum species such as a platinum(I) radical upon UV irradiation, and the radical may react with the substrate, by way of, for instance, polymerization such as radical polymerization, crosslinking reaction, etc. to form a reaction adduct, in particular a Pt-substrate adduct.

Depending on the nature of the substrate, the Pt-substrate adduct as formed may be different. In an embodiment, the substrate may be a hydrogel-based substrate such as polyacrylamide or gelatin. In another embodiment, the substrate may be a biomolecule-based substrate such as an amino acid or polypeptide. That said, in these embodiments, the Pt-substrate adduct may be any one of a Pt-polyacrylamide, Pt-gelatin, Pt-amino acid or Pt-protein adducts.

The Pt(IV) complex as descried herein is particularly a photoactivatable Pt(IV) complex. The term “photoactivatable Pt(IV) complex” as used herein refers to a platinum complex with a Pt core having an oxidation state of +4, and the complex may be activated to generate reactive species, particularly including reactive radical species, by way of photolysis upon exposure to radiation particularly UV radiation. In other words, the Pt(IV) may be photoactivated upon UV radiation and to generate photolyzed active species such as reactive radical species including a platinum(I) radical as mentioned.

The platinum(IV) complex may have a structure of Formula (Ia):

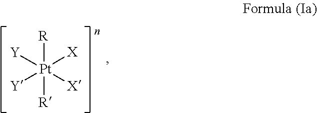

wherein X, X′, Y, Y′, R and R′ are electron donor ligands; and n is zero, any positive charge or negative charge.

An “electron donor ligand” means a ligand which is electron donating, i.e. has a donor atom with electron-donating ability such as a nitrogen atom, an oxygen atom, a phosphorous (P) atom or a sulfur atom. Examples may include nitrogen-containing ligands, oxygen-containing ligands, phosphorous-containing ligands, sulfur-containing ligands and halogen containing ligands. In particular, the electron donor ligand may include, for example, imine, aqua, halido (i.e. halide ions in particular including chlorido, bromido or fluorido), amines, diamines, triamines, ammine (NH₃), alkyl, cyanido, nitrato, hydroxido, alkoxy, phenoxy, anions of an alkyl mono- or poly-, such as di-, carboxylic acid such as oxalato or dianions of glycolic acid, an alcoholato ligand, alkylthio, thiolato, phosphito, phosphane, β-diketone, nitrato, a heterocycle such as pyridine or 2-methylpyridine. The term “alkyl” as used herein refers to saturated, straight-chain or branched hydrocarbons which may, for example, contain between 1 and 20 carbon atoms such as 1 to 5 carbon atoms.

In an embodiment, the platinum(IV) complex may have at least one of the nitrogen-containing ligands, oxygen-containing ligands, phosphorous-containing ligands, sulfur-containing ligands or halogen-containing ligands as mentioned above.

In a particular embodiment, the platinum(IV) complex may have a structure of Formula (Ib):

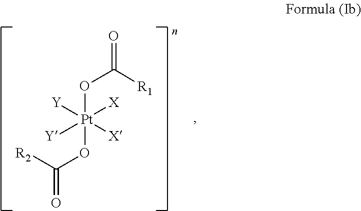

wherein:
X, X′, Y, and Y′ are independently selected from the group consisting of ammonia, halide, oxalate, diamines, dicarboxylate, and glycolate; R₁ and R₂ are independently selected from the group consisting of a linear or branched C1-C4 alkyl chain, a carboxyl group, and a carboxylate ester-containing group; and n is zero charge. The term “halide” generally denotes halide ions, in particular, including chloride, bromide, or fluoride.
R₁ and R₂, in particular, are independently selected from the group consisting of —CH₃, —CH₂CH₃, —CH₂CH₂CH₃, —CH₂COOH, —CH₂CH₂COOH, —CH₂COOCH₃, —CH₂CH₂COOCH₃, and —CH₂COOCH₂CH₃. That said, the innocent axial ligands of the Pt(IV) complex may be one of a carboxylic acid, succinic acid, adipic acid, succinic ester, or adipic ester. It is believe that these ligands would lack a strong absorbance at 365 nm, thus minimizing their competitive absorption of UV radiation at 365 nm with the Pt core, thereby facilitating the generation of the reactive radical species as mentioned herein, by way of, for example, charge-transfer such as ligand-to-metal charge transfer (LMCT) process. In particular embodiments, R₁ and R₂ may be independently selected from the group consisting of —CH₃, —CH₂CH₂COOH, or —CH₂CH₂COOCH₃, and —CH₂COOCH₂CH₃.

In an embodiment, X and X′ may be linked to form a first bidentate ligand. In a particular embodiment, the first bidentate ligand may be cyclobutane dicarboxylate and/or oxalate, Y and Y′ may be ammonia and/or chloride, R₁ and R₂ are —CH₃, —CH₂CH₂COOH or —CH₂CH₂COOCH₃; and n is zero charge.

In another embodiment, Y and Y′ may be linked to form a second bidentate ligand. In a particular embodiment, the second bidentate ligand may be a diamine; X and X′ may be ammonia and/or chloride, R₁ and R₂ are —CH₃, —CH₂CH₂COOH, or —CH₂CH₂COOCH₃; and n is zero charge.

As specific embodiments, the platinum(IV) complex has a structure of Formula (IIa), Formula (IIb), Formula (IIc), Formula (IId), Formula (IIe), or Formula (IIf):

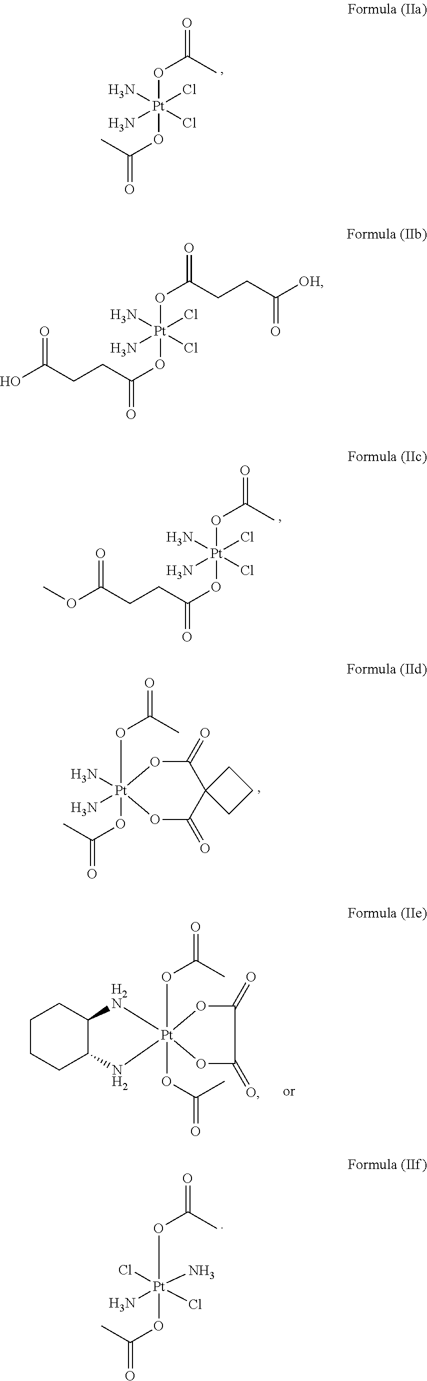

As mentioned, the Pt(IV) complexes of the present invention are photoactivatable to generate reactive species in the presence of radiation but being chemically unchanged in the absence of radiation. For example, in an embodiment where the Pt(IV) complex may comprise a cisplatin core, such as the platinum(IV) complex having a Formula (IIa), the structure of the complex may remain unchanged for at least 30 min in the absence of radiation, but being arranged to generate one or more of acetate radical, cisplatin, chlorine radical, ROS and the photolyzed active platinum species upon photoexcitation. In particular, the Pt(IV) complex having a structure of Formula (IIa) may be excited to the transition state via ligand-to-metal charge transfer (LMCT). Then, each acetato ligand may donate one electron to the Pt(IV) center, resulting in homolytic cleavage of the Pt—O bond and subsequent reductive elimination, yielding cisplatin and acetate radicals. The latter may further generate ROS in the presence of oxygen and water. These reactive radical species may then attack the cisplatin, yielding various active platinum species (including platinum(I)) and chlorine radicals.

As mentioned, the photolyzed active platinum species generated from the platinum(IV) complex would react with the substrate such as the hydrogel-based or biomolecule-based substrate as defined herein to form various Pt-based adduct. For example, in a specific embodiment where the substrate is a hydrogel-based substrate of polyacrylamide, such as a hydrogel-based substrate comprising 10% acrylamide/bis-acrylamide (29:1), the Pt(IV) complex may act as a photoinitiator by generating photolyzed active platinum species such as the platinum(I) radical and/or other radical species as described herein to induce hydrogelation/polymerization of acrylamide and bis-acrylamide, resulting in a Pt-gel adduct with a porous network and homogeneous microstructure. Advantageously, it is found that the hydrogel adduct may retain at least about 75% of the photolyzed Pt species and possesses antibacterial activity against E. coli (DH5α).

In another specific embodiment where the hydrogel-based is a hydrogel-based substrate of gelatin, such as 5% gelatin, the Pt(IV) complex may act as a photocrosslinker by generating photolyzed active platinum species such as the platinum(I) radical and/or other radical species as described herein to produce a gelatin hydrogel. In particular, the photolyzed Pt species may act a photocrosslinker to directly produce the gelatin hydrogel (i.e., Pt-gelatin adduct). That said, given that an additional (photo) crosslinker and/or (photo) initiator is not required, the gelatin hydrogel may be produced by way of a facile synthesis.

In a further specific embodiment, the Pt(IV) complex may act as a photoreactive labeling agent by generating photolyzed active platinum species such as the platinum(I) radical and/or other radicals as described herein to produce a Pt-amino acid adduct or a Pt-protein adduct. In particular, the photolyzed Pt species may crosslinks the amino acid, such as cysteine, methionine, tryptophan, and tyrosine or proteins, such as BSA upon photoexcitation of the Pt(IV) complex.

In an embodiment, the platinum(IV) complex may further comprise a functional moiety that is attached to the platinum(IV) complex for functionalizing the platinum-based adduct. The platinum(IV) complex, for example, may have a structure of Formula (IIIa):

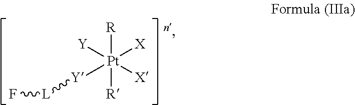

wherein:
X, X′, Y, Y′, R and R′ are as defined herein;
L is a linker group;
F is the functional moiety for functionalizing the platinum-based adduct; and
n′ is zero, any positive charge or negative charge.

The term “functional moiety” as used herein refers to a functional part of the platinum(IV) complex which may allow the complex to add new functions, features, and/or properties to (i.e., functionalizing) a material such as the platinum-based adduct as mentioned herein. The functional moiety may be, for example, a reactive group that is complementary to the reactive group of another molecule such as a chemical reporter molecule (e.g. fluorescent reporter/fluorophore, MRI reporter, etc.), a drug molecule, or biomolecules (e.g., biotin, peptides such as cell penetrating peptides and the like).

The linker group L is an organic unit of any lengths comprising atoms or groups to link, i.e. to connect, two parts of the platinum(IV) complex, namely the ‘platinum part’ of the platinum complex and the ‘functional part’ F together. Examples may include a straight chain or branched alkanediyl or amidyl chain. In particular embodiments of the present invention, L may be an amidyl chain of —(CH₂)ₘCONH—, wherein m is an integer which is >0 such as 1-6.

In a particular embodiment, the platinum(IV) complex may have a functional group having a reactive group that is complementary to the reactive group of a fluorescent reporting unit. The phrase “complementary” generally denotes that the reaction between two reactive groups occurs in pair and such reaction would not occur without the presence of one another reactive groups. Examples of such kind may include, redox reaction between reductant and oxidant, bioorthogonal reaction, reaction between antibodies and antigens, etc. The terms “fluorescent reporting unit” or “fluorescent reporter” refer to a fluorescent compound or molecule that emits a radiation at a particular wavelength or a particular range of wavelengths upon photoexcitation, and may emit a radiation at another wavelength and/or intensity upon reacting with the functional moiety of the Pt(IV) complex, where such change of emission wavelength and/or intensity may reflect the properties, such as reaction rate, reaction yield/efficiency, etc. of the Pt(IV) complex. Examples of fluorescent report unit may include fluorescein, rhodamine, cyanine dyes, BODIPY dyes, and the like.

Preferably, the platinum(IV) complex may have a structure of Formula (IIIb):

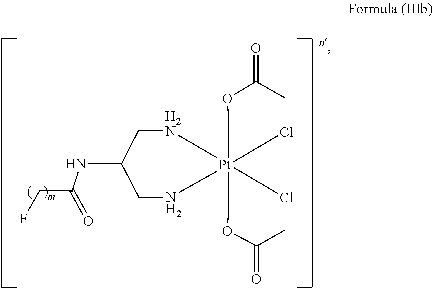

wherein:
m is 1-6;
F is the functional moiety which is a biorthogonal group selected from the group consisting of azide, terminal alkyne, activated cyclooctyne, and tetrazine;
n′ is zero charge.

In a specific embodiment, the platinum(IV) complex may have a structure of Formula (IV):

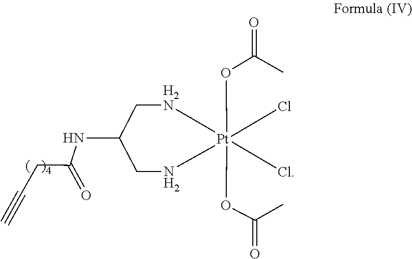

The method of preparing the platinum-based adduct is described below. The method may comprise the steps of:
providing a solution mixture comprising a hydrogel precursor or a biomolecule, and a platinum(IV) complex having a structure of Formula (Ia) as defined herein; and
administering to the solution mixture radiation in an amount and of a wavelength effective to activate the platinum(IV) complex to form a photolyzed platinum species comprising a platinum(I) radical, which reacts with the hydrogel precursor or the biomolecule to form the platinum-based adduct.

The hydrogel precursor may be prepared by providing a monomer solution. Optionally or additionally, the monomer solution may further include a crosslinking agent. For example, in an embodiment where the platinum-based adduct is a Pt-hydrogel adduct such as Pt-polyacrylamide adduct, the hydrogel precursor may be a solution such as an aqueous solution containing such as 10% of acrylamide (i.e., monomer) and bis-acrylamide (i.e. crosslinking agent) in a ratio of, for example 29:1. In another example embodiment where the Pt-based adduct is a Pt-gelatin adduct, the hydrogel precursor may be an aqueous solution of 5% gelatin (i.e., monomer).

In an embodiment where the platinum-based adduct is a Pt-biomolecule adduct such as a Pt-amino acid or a Pt-BSA adduct, the solution mixture may be a buffer solution such as a PBS solution at pH about 7.4 containing the amino acid or BSA. In particular, the method of this embodiment may further comprise the step of incubating the solution mixture in the dark at 37° C. for about 1 hour prior to proceed to the subsequent irradiation step.

The concentration of platinum(IV) complex present in the solution mixture may be varied according to practical needs. In particular, the concentration of the platinum(IV) complex may be from about 10 μM to about 1000 μM.

After the solution mixture is prepared, radiation may be administered to the solution mixture with an effective wavelength and/or amount to generate reactive radical species as described herein. Preferably, the radiation is UV radiation, particularly UV radiation having a wavelength of about 360 nm to about 370 nm, such as about 365 nm. The UV radiation may be applied to the solution mixture at a power density from about 9 mW/cm² to about 10 mW/cm², such as about 9.8 mW/cm².

The UV radiation may be applied to the solution mixture for about 5 mins to about 10 mins, about 4 mins to about 11 mins, about 6 mins to about 10 mins, about 8 mins to about 10 mins or particularly 10 mins.

Optionally or additionally, the as-formed adduct may be isolated and/or washing with suitable solvents. The expression “isolated” means separating the Pt-based adduct from the bulk reaction mixture/system, such as separating the Pt-hydrogel from a mold or container, precipitating the Pt-protein adduct from the reaction mixture, etc.

Another aspect of the invention relates to a photoinitiator for hydrogelation comprising a photolyzed active platinum species generated from a platinum(IV) complex having a structure of Formula (Ia):

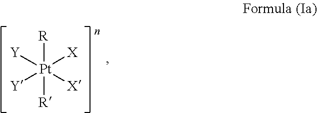

wherein X, X′, Y, Y′, R and R′ are electron donor ligands as defined herein; and n is zero, any positive charge or negative charge.

In an embodiment, the platinum(IV) complex has a structure of Formula (Ib):

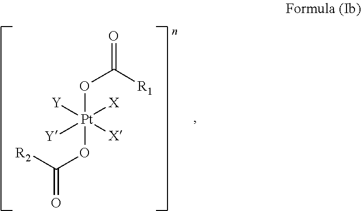

with X, X′, Y, and Y′, R₁, R₂, and n being defined herein.

In particular, as mentioned, in an embodiment where the substrate is a hydrogel-based substrate, about 75% of photolyzed platinum species may be retained in the adduct after the complex is exposed to radiation such as UV radiation at about 365 nm.

As a specific embodiment, the platinum(IV) complex has a structure of Formula (IIa):

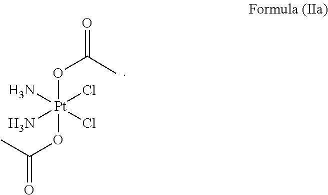

The present invention also relates to a photocrosslinker for hydrogelation or protein labeling comprising a photolyzed active platinum species generated from a platinum(IV) complex having a structure of Formula (Ia) or Formula (IIIa):

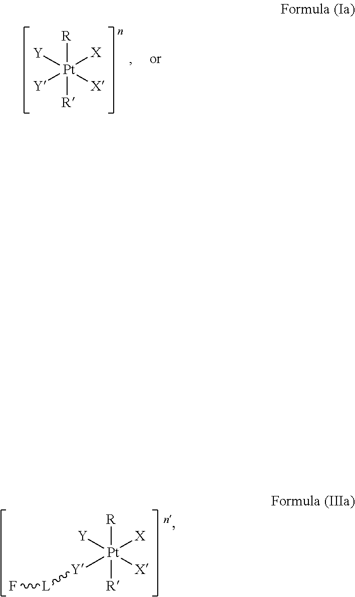

wherein:
X, X′, Y, Y′, R and R′ are electron donor ligands as defined herein;
L is a linker group as defined herein;
F is a functional moiety for protein labeling; and
n and n′ are each independently being zero, any positive charge or negative charge.

In an embodiment, the platinum(IV) complex, particularly the platinum(IV) complex that acts as a photocrosslinker for hydrogelation, may have a structure of Formula (Ib):

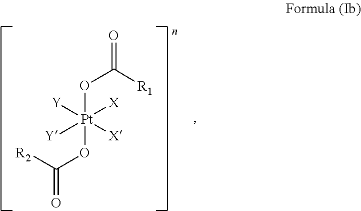

with X, X′, Y, Y′, R₁R₂, and n being defined herein.

As a specific embodiment, the platinum(IV) complex has a structure of Formula (IIa):

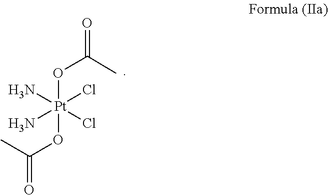

In another embodiment, the platinum(IV) complex, particularly the platinum(IV) complex that acts as a photocrosslinker for protein labeling, may have a structure of Formula (IIIb):

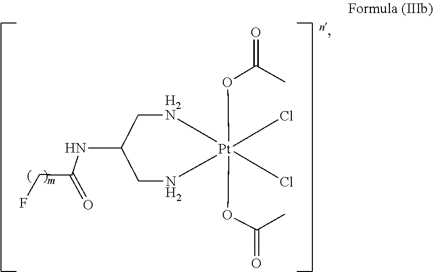

wherein:
m is 1-6;
F is the functional moiety which is a biorthogonal group selected from the group consisting of azide, terminal alkyne, activated cyclooctyne, and tetrazine; and
n′ is zero charge.

As a specific embodiment, the platinum(IV) complex has a structure of Formula (IV):

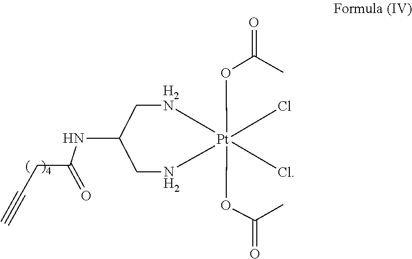

Further pertaining to the present invention is an antibacterial platinum-based adduct comprising a substrate to which a photolyzed active platinum species is attached, the photolyzed active platinum species is generated from a platinum(IV) having a structure of Formula (IIa):

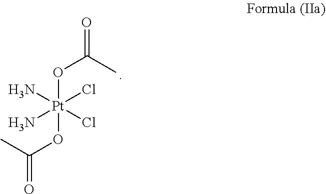

In an example embodiment, the substrate may comprise polyacrylamide and the adduct may have an antibacterial activity against E. coli (DH5α), particularly when about 1 mM of the platinum(IV) complex is used for the preparation of the adduct.

Hereinafter, the present invention is described more specifically by way of examples, but the present invention is not limited thereto.

### EXAMPLES

### Materials, Instruments, and Reagents

Unless otherwise noted, all the reactions were carried out under normal atmospheric conditions with protection from light. All chemicals and solvents were purchased from commercial resources. Dulbecco's Modified Eagle Medium (DMEM), Minimum Essential Medium (MEM), Roswell Park Memorial Institute (RPMI) 1640 medium, trypsin, phosphate-buffered saline (PBS), and fetal bovine serum (FBS) were obtained from Life Technologies.

Unless otherwise noted, the light source is a LED UV light (365 nm, 9.8 mW/cm²). NMR data were recorded with Bruker AVANCE III 300 MHZ, 400 MHZ, and 600 MHz spectrometers at room temperature with protection from light. ESI-MS data were recorded with a Liquid Chromatography-Mass Spectrometer (API-3200 Triple-Q MS/MS). High-resolution MS data were recorded with Bruker Daltonics microTOF and Sciex X500R Q-TOF.

HPLC analysis was carried out by Phenomenex 00G-4435-E0 Gemini 5 μm C18 110 Å 250 mm×4.6 mm HPLC Column on a Shimadzu LC-20A HPLC instrument. The samples were monitored by UV absorbance at 254 nm and ESI-HRMS at scan mode. Solvent A (H₂O) and Solvent B (acetonitrile) were used for a gradient elution at a flow rate of 1 mL/min. The samples were eluted by using a program as follows: 0% B (0 min)—100% B (9 min)—100% (11 min)—0% (12 min)—0% (16 min).

Pt content was measured by an Inductively Coupled Plasma-Optical Emission Spectrometer (ICP-OES) (PE Optima 8000) or an inductively Coupled Plasma-Mass Spectrometer (ICP-MS) (PE Nexion 2000). Gel fluorescence scanning was performed with Fujifilm FLA9000.

### Methods and Characterization

### Photolysis of Pt Complexes

The photolysis of Pt complexes in test tubes was examined by reversed-phase high performance liquid chromatography (RP-HPLC). 1 mM Pt complexes were dissolved in water. Then the mixed solution was irradiated with UV light (365 nm, 9.8 mW/cm²) for 10 min at room temperature, and the solution was examined by HPLC. The percentage of remained Pt complexes was obtained from the ratio of the peak area of Pt complexes upon irradiation to that of the intact Pt complexes.

### Radical Spin Trap Assay

1 mM cisPt(IV)-1 (Pt(IV) complex of Formula (IIa)) and cisplatin in water was mixed with 1 or 10 mM DMPO or TEMPO. The mixed solution was irradiated with UV or kept in the dark for 10 min. The solution was analyzed by HPLC. The Pt(I) adduct products were characterized by ESI-HRMS, and the acetate radical adduct was separated and characterized by proton NMR. For the study of photolysis rate, the solution was irradiated with UV (365 nm, 9.8 mW/cm²) for 0.5, 1, 2, 3, 4, 5 and 10 min, with or without the radical spin trap and analyzed by HPLC. For the study of dark stability, the solution was incubated at room temperature in the dark for 30 min before examined by HPLC.

### Electron Paramagnetic Resonance (EPR) Analysis

EPR spectra were recorded with an ADANI SpinscanX spectrometer, operating at 100 kHz field modulation using the 150 μT modulation amplitude. 5,5-dimethyl-1-pyrroline N-oxide (DMPO) and 2,2,6,6-tetramethylpiperidine (TEMP) were chosen as the spin trap. Typically, 500 mM DMPO or TEMP was mixed with 25 mM cisPt(IV)-1 in water. The mixture was then irradiated with UV or kept in the dark for 10 min at room temperature. The samples were transferred to a quartz capillary, and the EPR spectra were recorded at room temperature.

### Reactive Species Detection by Probes

500 UM of Pt complexes were mixed with 250 μM radical probes: N,N-diethyl-p-phenylenediamine (DPD) (for the detection of chlorine radicals), methylene blue (MB) (for the detection of ROS, chlorine, and hydroxyl radicals), 2,3-diaminophenazine (OPD) (for the detection of ROS and hydroxyl radicals), 3,3′,5,5′-tetramethylbenzidine (TMB) (the detection of ROS, hydroxyl radicals) in H₂O. The mixtures were irradiated with UV (365 nm, 9.8 mW/cm²) for 10 min or kept in the dark. The absorbance of probes was recorded by the plate reader.

### Total ROS Generation in Solution Detected by DCFH

20 μL of dichlorodihydrofluorescein diacetate (DCFH2-DA) (1 mM) were added to 80 μL of sodium hydroxide in water (10 mM) and stirred for 30 min at room temperature in the dark to obtain DCFH2. Next, 400 μL of phosphate-buffered saline (pH 7.4) was added to the mixture to obtain a DCFH2 solution (40 μM). Then 5 μM DCFH2 was added to 50 μM Pt complexes in PBS buffer, followed by irradiation with UV for 10 min or kept in the dark. For the hypoxia experiment, the complex solution was deoxygenated by infusion of argon gas for 15 min. The fluorescence was monitored by the plate reader with the excitation at 450 nm, and emission at 490-700 nm.

### Quantification of Cisplatin by LC-HRMS

1 mM cisPt(IV)-1 was added to different concentrations (0, 0.1, 1, 10, 50/100 mM) DMPO/TEMPO in water. The mixture was irradiated with UV light (365 nm, 9.8 mW/cm²) for 10 min at room temperature. After that, the solution was diluted times before being analyzed by the LC-HRMS. For the hypoxia experiment, the complex solution was deoxygenated by infusion of argon gas for 15 min. The major ion intensity of cisplatin at 300.9608 was selected for quantification, and the concentration of cisplatin was quantified from the peak area of the standard curve.

### Gelation Assay

Hydrogelation experiments were carried out in glass vials. In a typical experiment, 100 μM of platinum complexes were added to 10% acrylamide/bis-acrylamide (29:1) in water. For hydrogelation with gelatin, 1 mM of platinum complexes was mixed with 5% gelatin in water. The mixture was irradiated with 365 nm light (9.8 mW/cm²) for 10 min at room temperature, and the formed hydrogel was tested by the vial inversion method.

For protein electrophoresis, 1 mM cisPt(IV)-1 was added to 10% acrylamide/bis-acrylamide (29:1), 375 mM Tris-HCl (pH 8.8), and 0.1% SDS in water. The mixture was irradiated at 365 nm for 10 min at room temperature to form the Pt-gel. Samples of 3 μL marker, 5 μg BSA, and 20 μg A2780 cell lysate were loaded. After separation, the gels were stained with Coomassie blue and imaged by a ChemiDoc Touch imaging system.

### Antibacterial Assay

100 μL hydrogel (0.1% 12959 or 1 mM cisPt(IV)-1+10% acrylamide/bis-acrylamide (29:1)) were prepared in a 96-well plate. The mixtures were irradiated (365 nm, 9.8 mW/cm²) for 20 min and washed with PBS twice. 20 μL of DH5α (around 9 107 CFU/mL) in PBS was added to the surface of the gel. Controls were carried out with 100 μM cisplatin in PBS or pure PBS concurrently. After incubation for 4 h at 37° C., the samples were washed with 100 μL PBS twice. The PBS were combined to collect the surviving bacteria and further diluted 10,000 times. 20 μL of bacteria in PBS were then evenly spread on the 10 cm agar plates and incubated for 18 h at 37° C., before being imaged by a ChemiDoc Touch imaging system. The optical density OD600 was also monitored every half an hour without or in the presence of Pt-gel treated with DH5α in LB medium with the microplate reader.

### Release Profile of Platinum in the Pt-Gel

500 μL Pt-gel was prepared by mixing 10% acrylamide/bis-acrylamide (29:1) and 1 mM cisPt(IV)-1 in water and irradiated with UV for 10 min in a 48-well plate. The Pt was washed with 1 mL PBS and transferred to a 15 mL tube. 5 mL PBS was added and the mixture was incubated at 37° C. with gentle shaking. 200 μL supernatant was taken at the given time points. The Pt concentration was ascertained by ICP-OES.

### BSA Binding

5 mg/mL BSA was mixed with Pt complexes (final Pt concentration=50 μM) in PBS (pH 7.4) buffer. The mixture was incubated for an hour at 37° C., prior to irradiation with UV or being kept in the dark for 10 min at room temperature. After that, acetone was added to the solution and kept at −20° C. for an hour to precipitate. The protein pellet was collected by centrifugation (14,000 rpm, 10 min at 4° C.) and resuspended in PBS buffer. The binding content of Pt was ascertained by ICP-OES.

In Vitro BSA Photolabeling 3 mg/mL BSA in PBS (pH 7.4) buffer was treated with different compounds at 10 μM (0.1% DMF). The mixture was incubated for 1 h at 37° C. prior to UV irradiation (365 nm, 9.8 mW/cm²) for 5 min at room temperature. The reaction was then quenched by the addition of acetone and stored at −20° C. for an hour. The precipitated protein pellet was then collected by centrifugation (14,000 rpm, 10 min at 4° C.). The samples were dissolved in PBS, followed by the addition of a freshly pre-mixed click chemistry reaction cocktail (100 μM TBTA, 1 mM CuSO₄, 1 mM TCEP, and 50 μM TAMRA-N₃). The reaction was incubated for an hour at ambient temperature before quenching with SDS-PAGE loading buffer. The labeled protein was separated by SDS-PAGE and visualized by in-gel fluorescence scanning.

For time-tracking analysis of the reaction, 3 mg/mL BSA in PBS was treated with Pt(IV)-based photo-crosslinker at 10 μM (0.1% DMF). 50 μL aliquot was taken every two minutes and precipitated with acetone. At given time points (5, 11, and 17 min), the reaction mixture was irradiated with UV for 30 s. The precipitated protein pellets were dissolved in PBS and analyzed by in-gel fluorescence scanning as described above.

### Example 1A

Synthesis of cisPt(IV)-1 (Pt(IV) Complex of Formula (IIa))

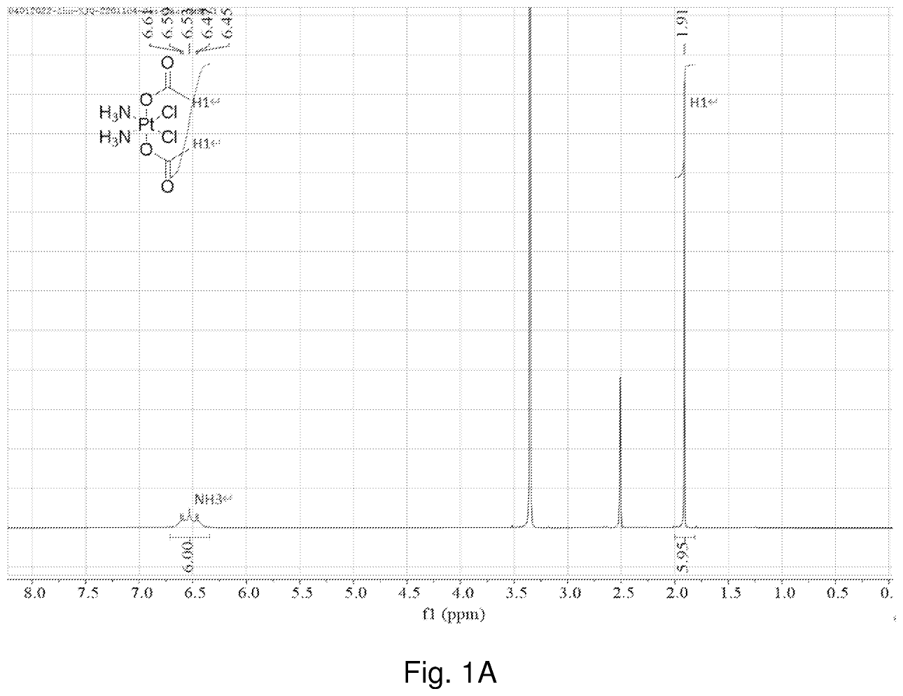

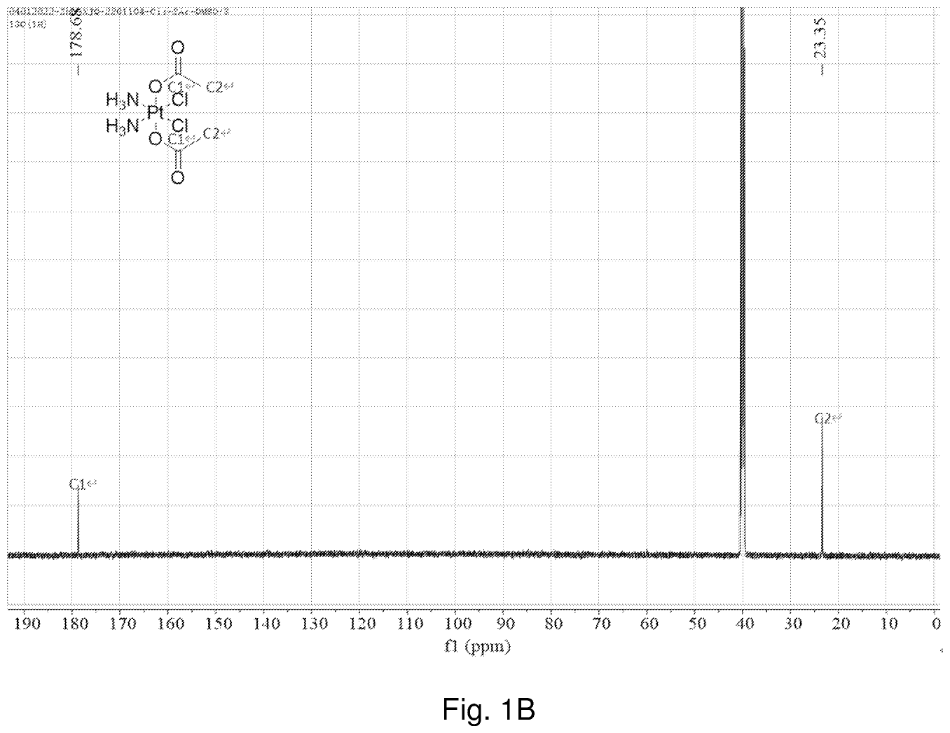

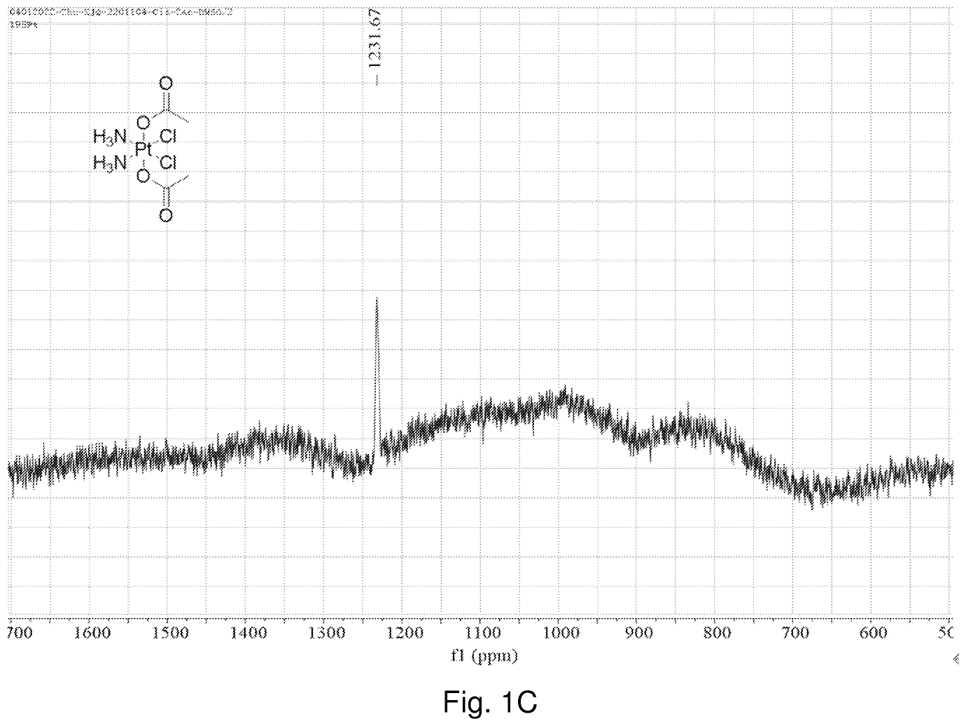

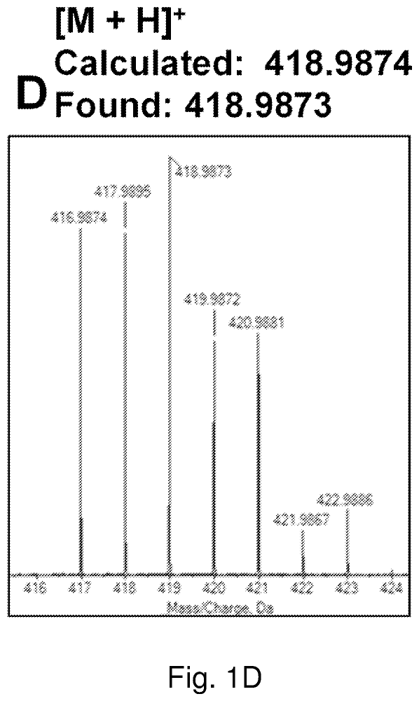

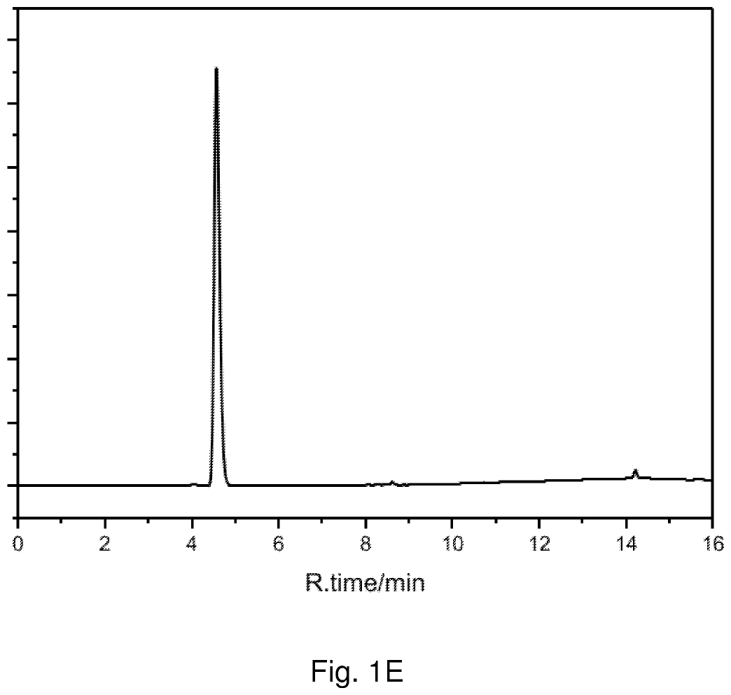

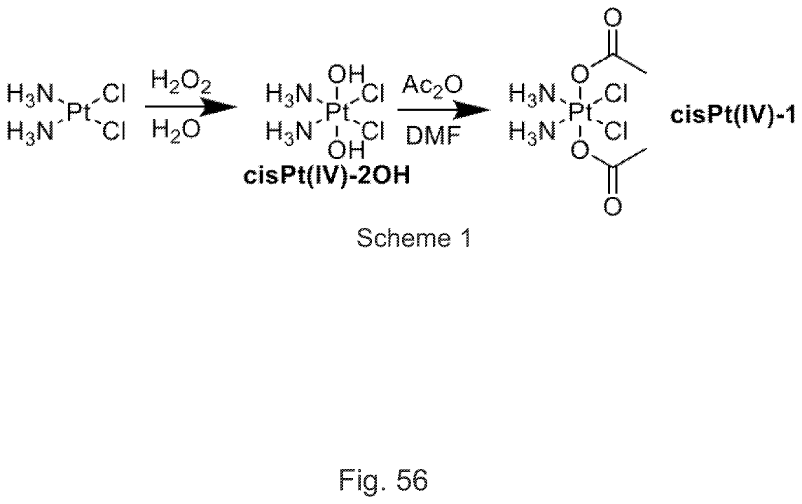

The synthesis of cisPt(IV)-1 is summarized in FIG. 56 (Scheme 1). Complex cisPt(IV)-2OH was synthesized according to reported method. 400 mg cisPt(IV)-2OH was suspended in 12 mL dry DMF. 170 μL acetic anhydride was added. The reaction was stirred for 24 h at room temperature. The solvent was removed by vacuum. The solid was washed with acetone and Et₂O and dried in vacuum to give the product. ¹H NMR (600 MHZ, DMSO-d6) § 6.53 (t, J=47.8 Hz, 6H), 1.91 (s, 6H) (FIG. 1A). ¹³C NMR (151 MHz, DMSO-d6) δ 178.68, 23.35. (FIG. 1B) ¹⁹⁵Pt NMR (129 MHz, DMSO-d6) δ 1231.67 (FIG. 1C). ESI-HRMS (positive mode) m/z: [M+H]⁺ calculated for C₄H₁₃Cl₂N₂O₄Pt: 418.9874, found: 418.9873 (FIG. 1D). HCLP chromatogram of the as-obtained cis-Pt(IV)-1 shows a single peak, suggesting the high purity of the complex (FIG. 1E).

### Example 1B

Synthesis of cisPt(IV)-2 (Pt(IV) Complex of Formula (IIb))

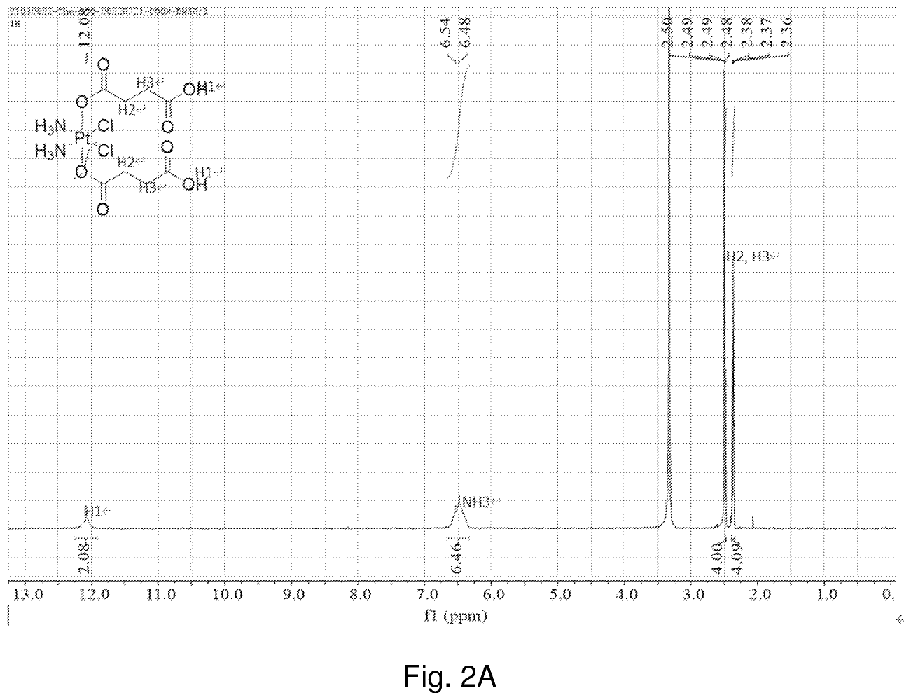

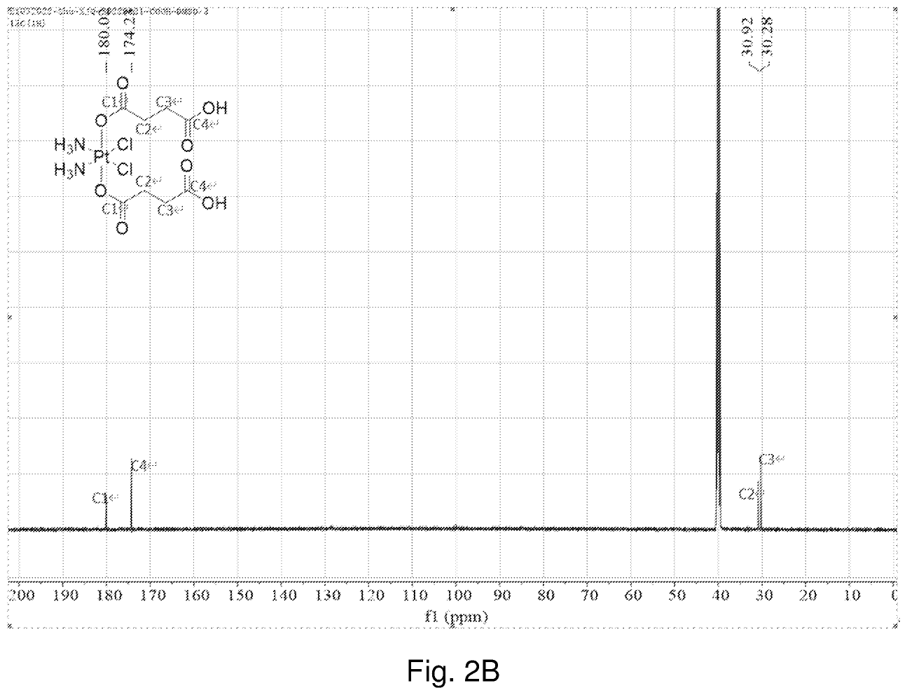

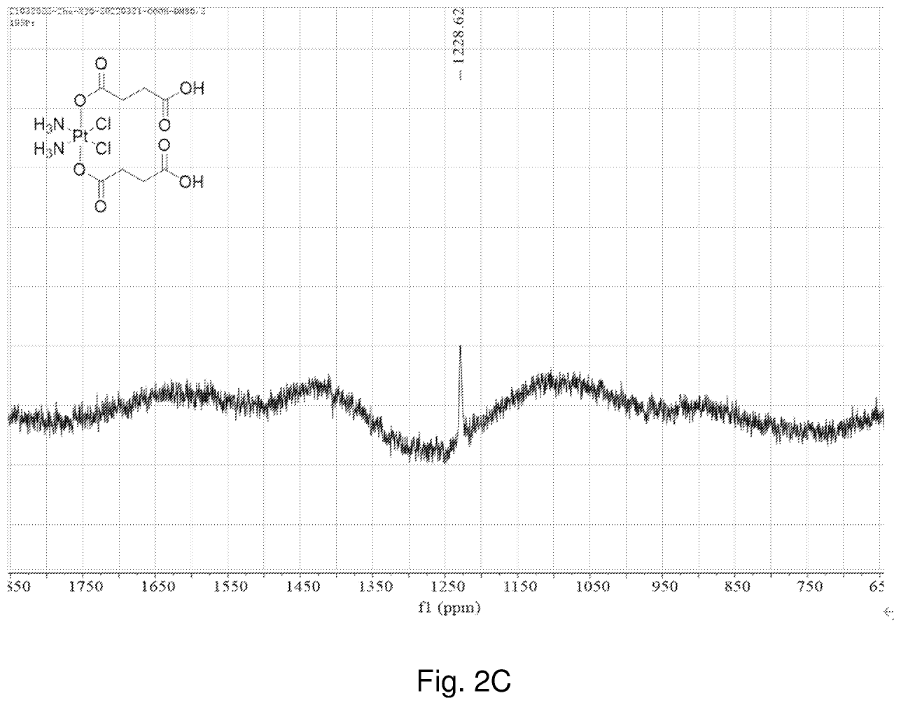

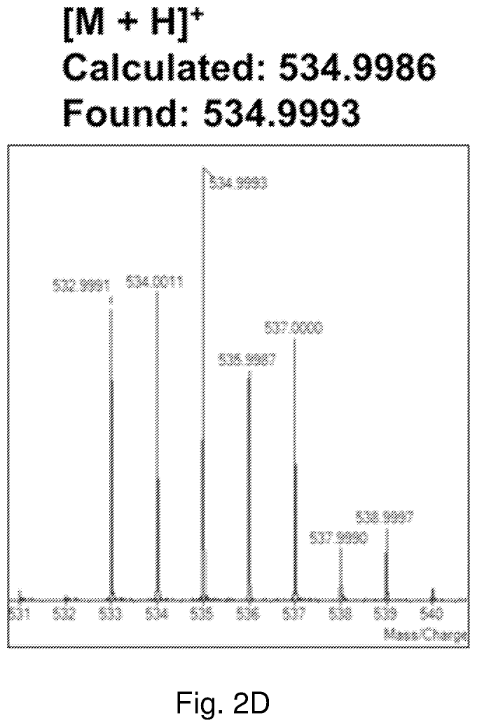

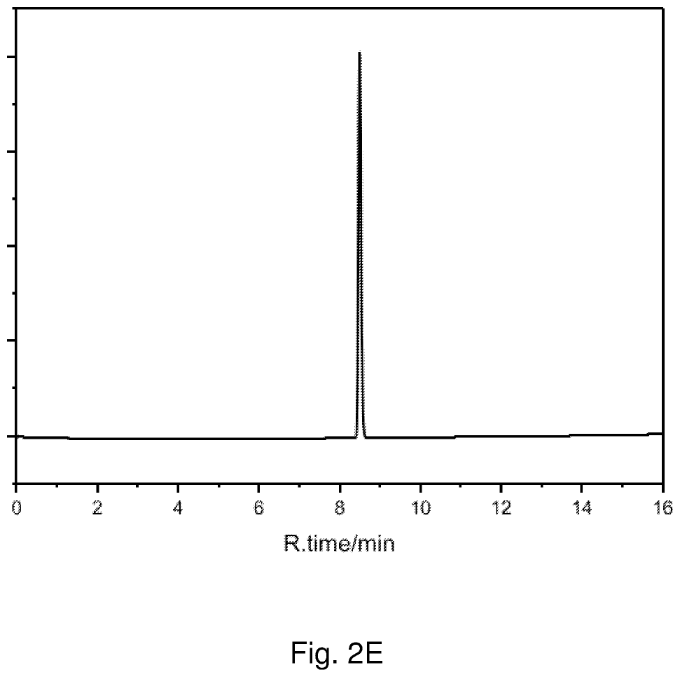

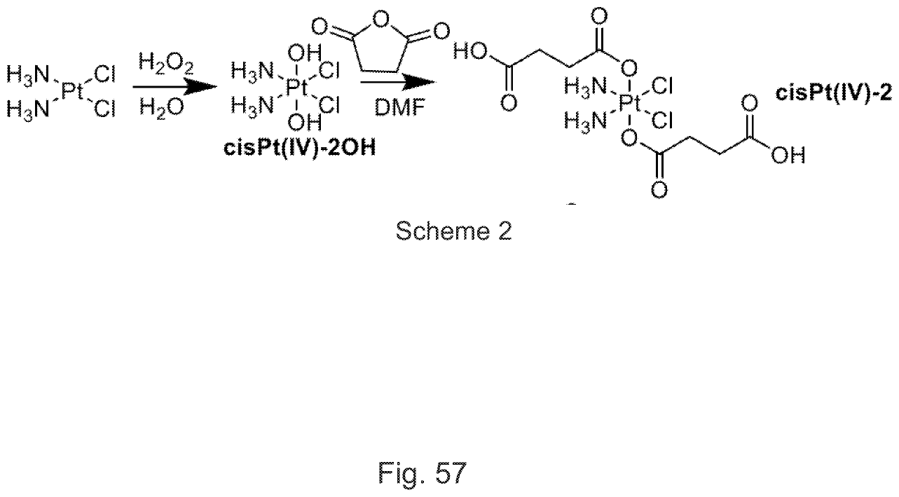

The synthesis of cisPt(IV)-2 is summarized in FIG. 57 (Scheme 2). 200 mg of cisPt(IV)-2OH and 216 mg of succinic anhydride were mixed in a 50 mL round bottom flask. The mixture was dissolved in 1.5 mL DMSO and further stirred at 80° C. for 3 h. 5 mL acetone was then added, followed by precipitation with 35 mL Et₂O. The solid was collected and washed with Et₂O, and finally dried in vacuum. ¹H NMR (600 MHZ, DMSO-d6) δ 12.08 (s, 2H), 6.48 (s, 6H), 2.50-2.47 (m, 4H), 2.37 (t, J=7.1 Hz, 4H) (FIG. 2A). ¹³C NMR (151 MHZ, DMSO-d6) δ 180.02, 174.23, 30.92, 30.28 (FIG. 2B). ¹⁹⁵Pt NMR (129 MHz, DMSO-d6) δ 1228.63 (FIG. 2C). ESI-HRMS (positive mode) m/z: [M+H]⁺ calculated for C₈H₁₇Cl₂N₂O₈Pt: 534.9986, found: 534.9993 (FIG. 2D). HCLP chromatogram of the as-obtained cis-Pt(IV)-2 shows a single peak, suggesting the high purity of the complex (FIG. 2E).

### Example 1C

Synthesis of cisPt(IV)-3 (Pt(IV) Complex of Formula (IIc))

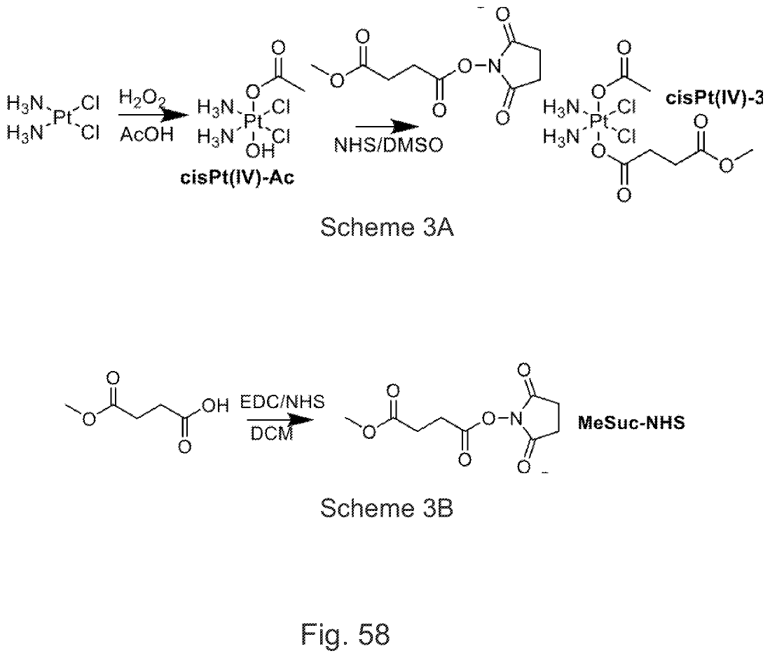

The synthesis of cisPt(IV)-3 is summarized in FIG. 58, top (Scheme 3A). As shown, the synthesis of cisPt(IV)-3 involves the reaction of cisPt(IV)-Ac with MeSuc-NHS, which is prepared as illustrated in FIG. 58, bottom (Scheme 3B).

Specifically, to a solution of 500 mg mono-methyl succinate in 35 mL DCM, 610 mg NHS and 1035 mg EDCI was added. The reaction mixture was stirred at room temperature for 6 h and quenched by the addition of water. The organic phase was washed with brine twice. The product was obtained by removing the solvent in vacuum and directly used without further purification.

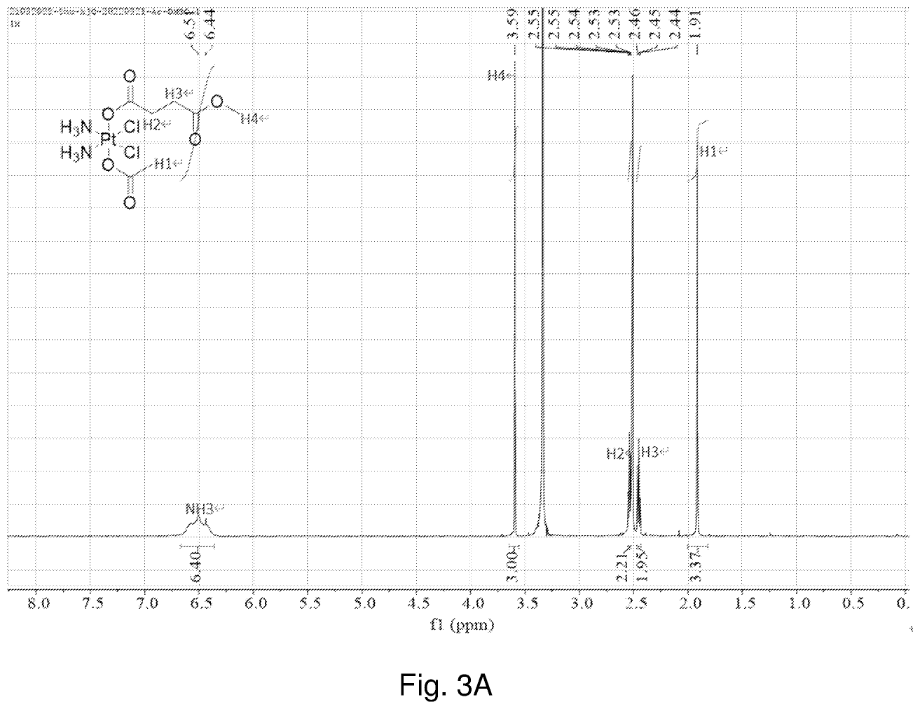

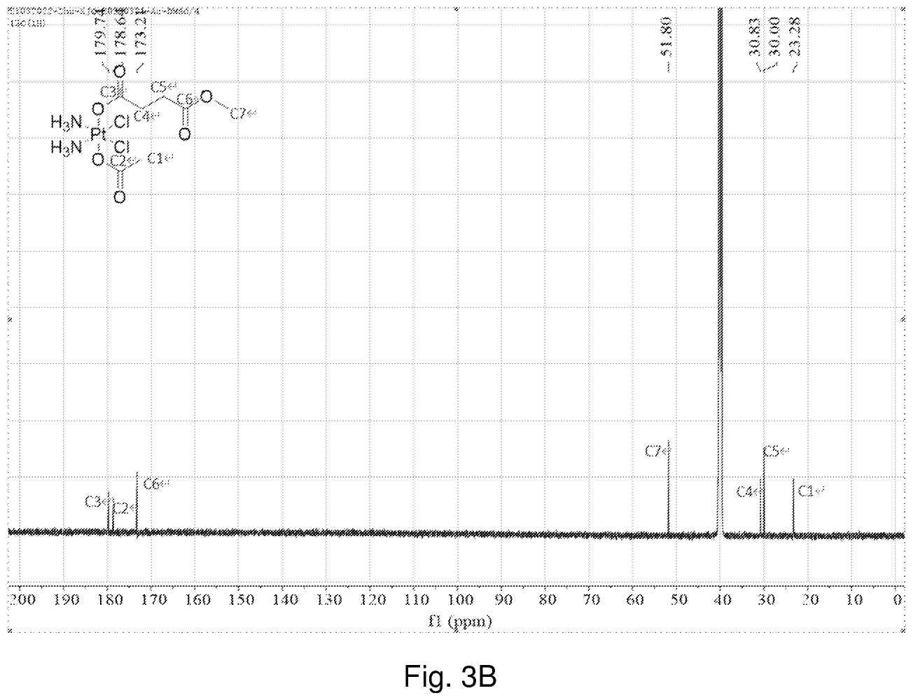

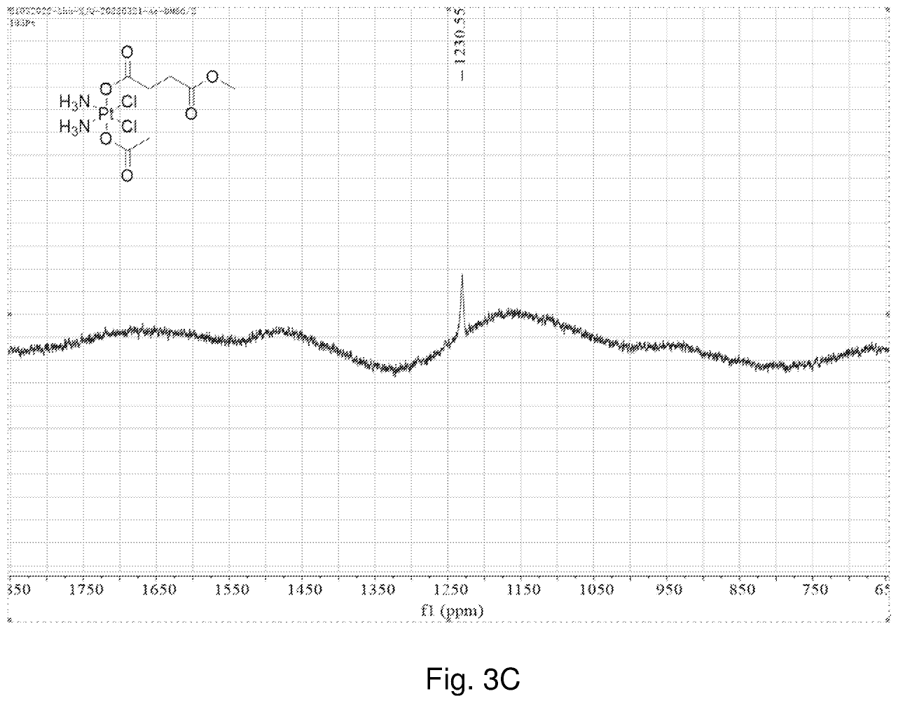

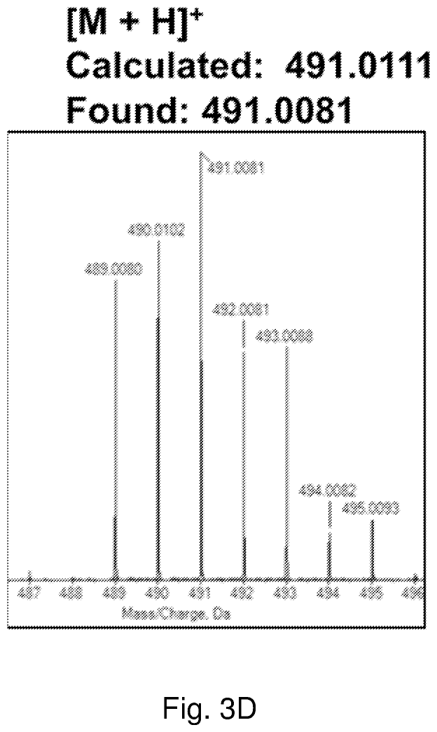

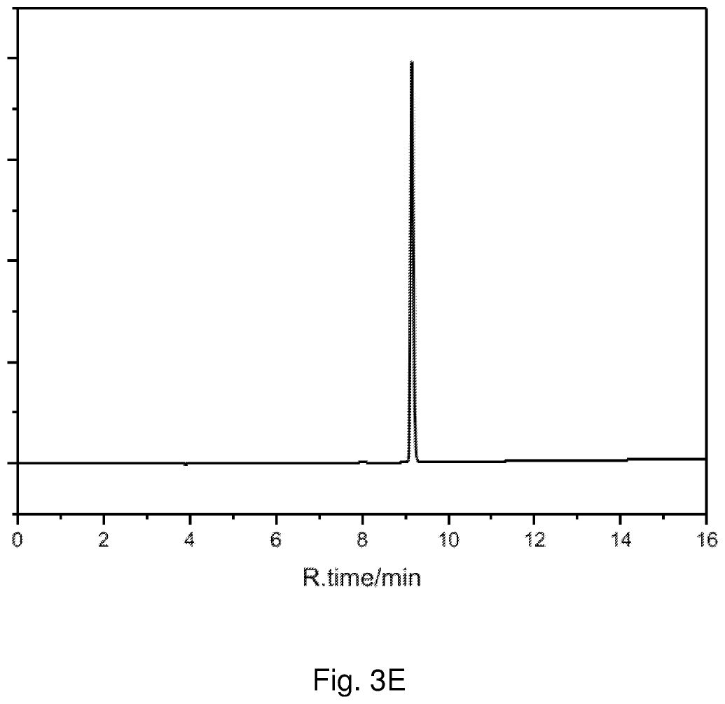

Complex cisPt(IV)-Ac was synthesized according reported method. Then, to a solution of 200 mg cisPt(IV)-Ac in 2 mL DMSO was added 122 mg MeSuc-NHS. The reaction mixture was stirred overnight at 45° C. The desired product was purified by HPLC. ¹H NMR (600 MHZ, DMSO-d6) δ 6.47 (d, J=43.7 Hz, 6H), 3.59 (s, 3H), 2.55-2.52 (m, 2H), 2.45 (t, J=7.0 Hz, 2H), 1.91 (s, 3H) (FIG. 3A). ¹³C NMR (151 MHZ, DMSO-d6) δ 179.74, 178.64, 173.21, 51.80, 30.83, 30.00, 23.28 (FIG. 3B). ¹⁹⁵Pt NMR (129 MHz, DMSO-d6) δ 1230.55 (FIG. 3C). ESI-HRMS (positive mode) m/z: [M+H]⁺ calculated for C₇H₁₇Cl₂N₂O₆Pt: 491.0111, found: 491.0081 (FIG. 3D). HCLP chromatogram of the as-obtained cis-Pt(IV)-3 shows a single peak, suggesting the high purity of the complex (FIG. 3E).

### Example 1D

Synthesis of carPt(IV) (Pt(IV) Complex of Formula (IId))

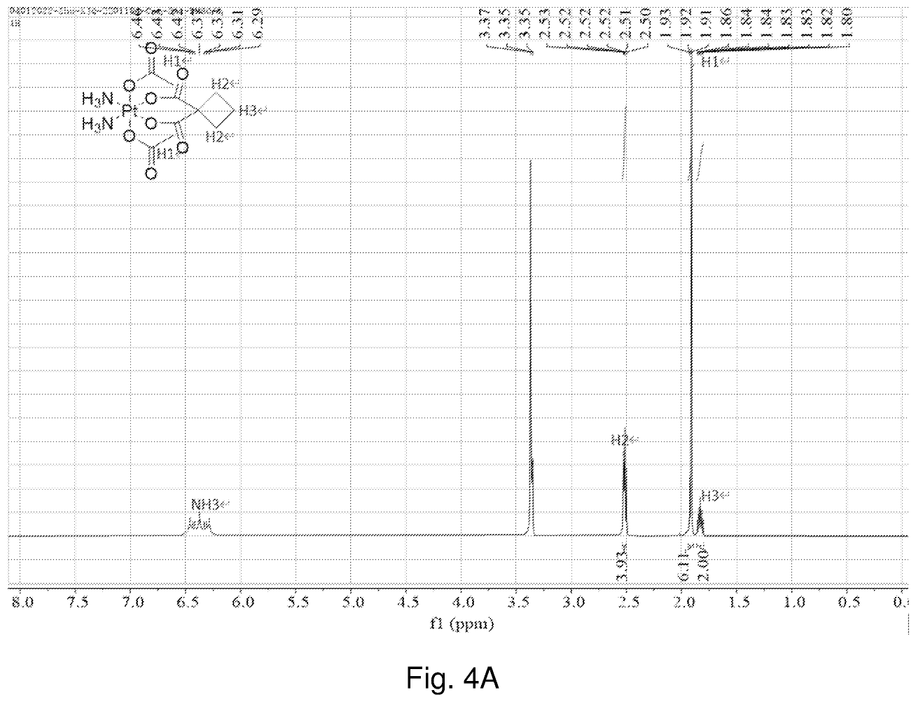

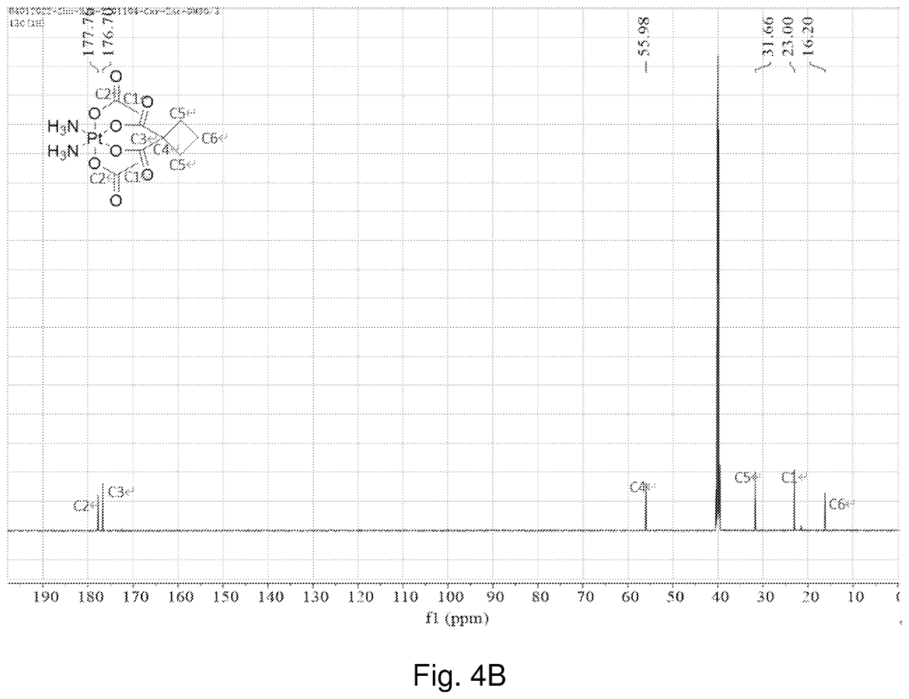

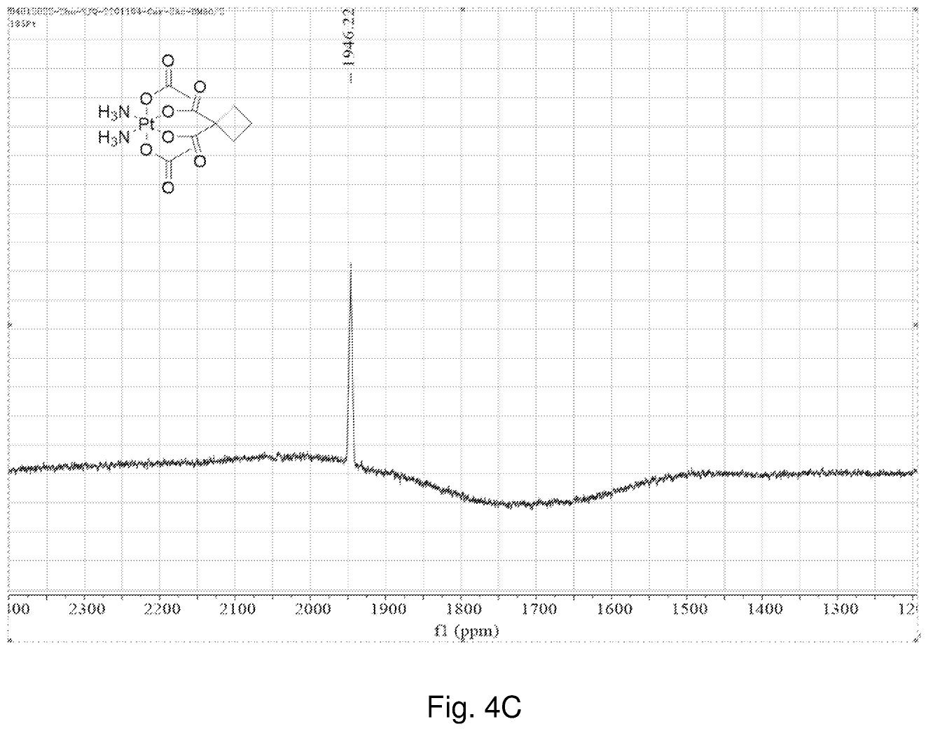

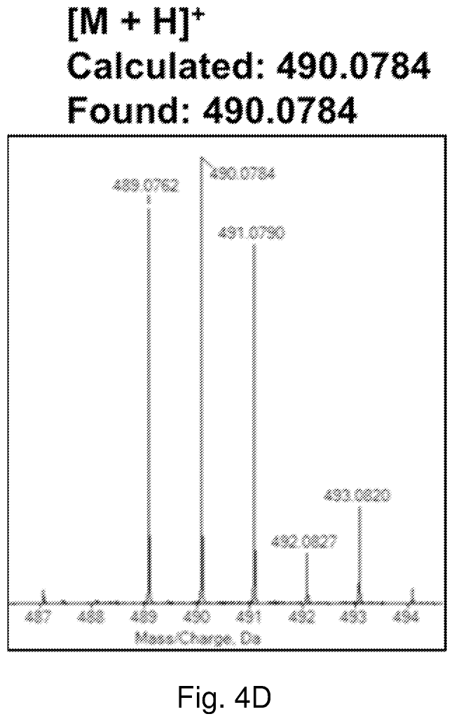

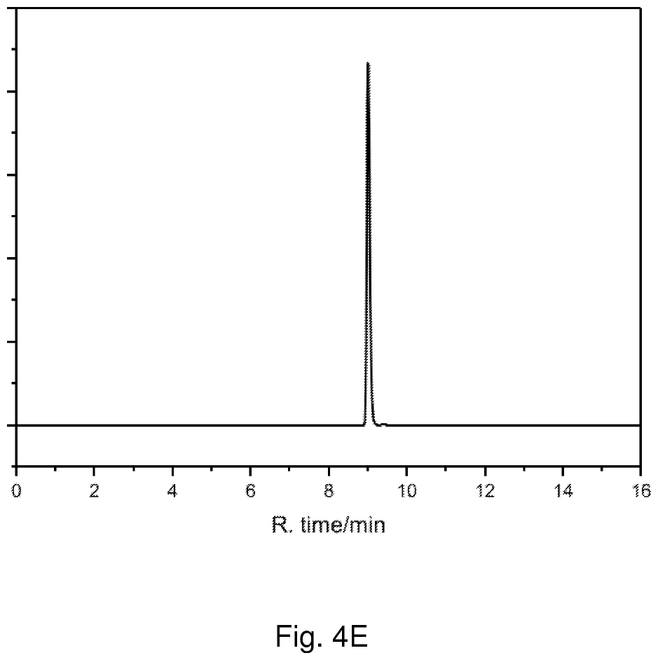

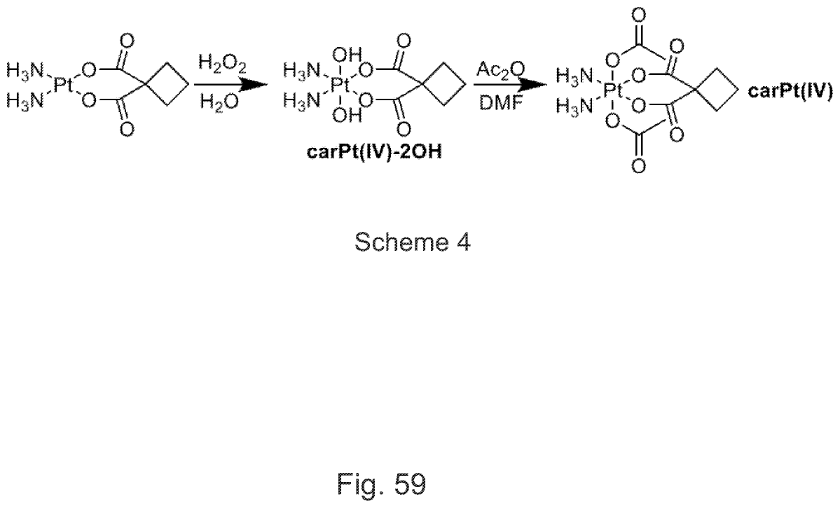

The synthesis of carPt(IV) is summarized in FIG. 59 (Scheme 4). Complex carPt(IV)-2OH was synthesized according to reported method. 400 mg carPt(IV)-2OH was suspended in 3 mL acetic anhydride. The reaction mixture was stirred overnight at room temperature. Et₂O was then added to the precipitate, and the product was collected, further washed with EtOH and Et₂O, and dried in vacuum. ¹H NMR (600 MHZ, DMSO-d6) δ 6.57-6.13 (m, 6H), 2.53-2.50 (m, 4H), 1.91 (s, 6H), 1.86-1.80 (m, 2H) (FIG. 4A). ¹³C NMR (151 MHz, DMSO-d6) δ 177.76, 176.70, 55.98, 31.66, 23.00, 16.20 (FIG. 4B). ¹⁹⁵Pt NMR (129 MHz, DMSO-d6) δ 1946.22 (FIG. 4C). ESI-HRMS (positive mode) m/z: [M+H]⁺ calculated for C¹⁰H₁₉Cl₂N₂O₈Pt: 490.0784, found: 490.0784 (FIG. 4D). HCLP chromatogram of the as-obtained carPt(IV) shows a single peak, suggesting the high purity of the complex (FIG. 4E).

### Example 1E

Synthesis of oxaPt(IV) (Pt(IV) Complex of Formula (IIe))

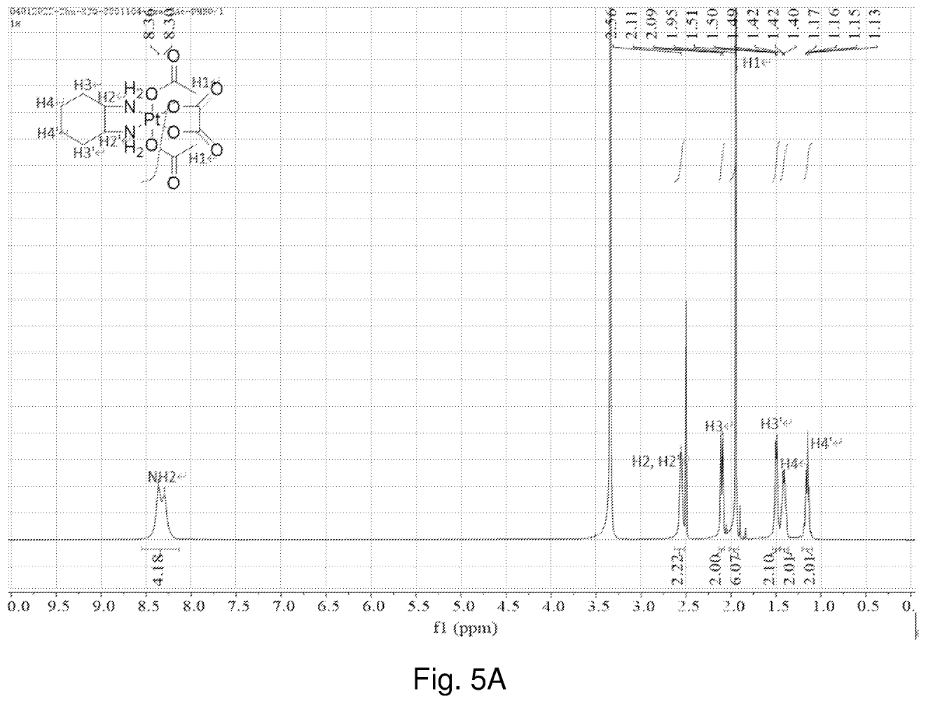

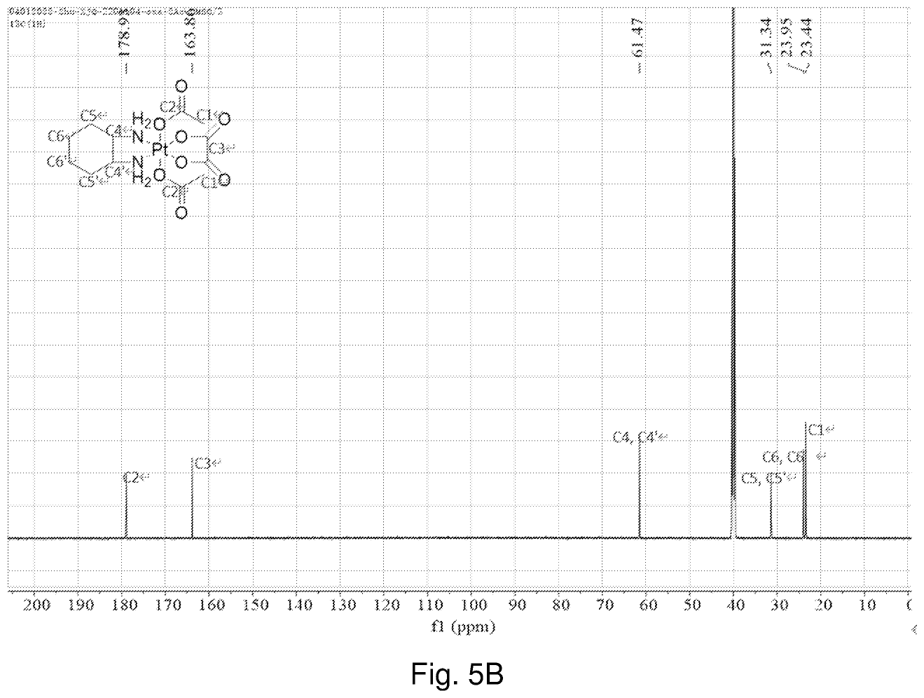

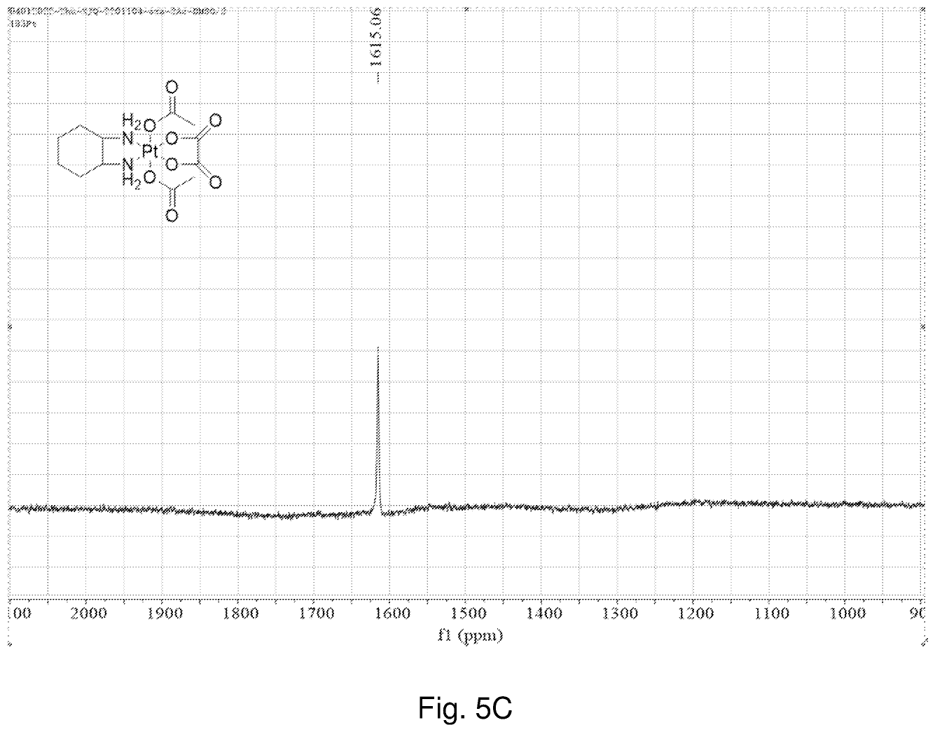

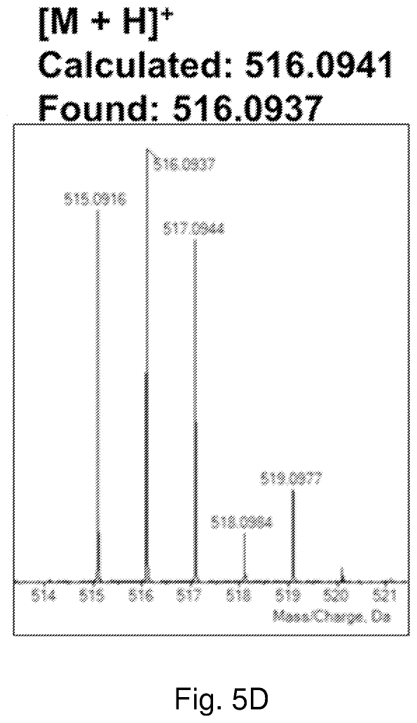

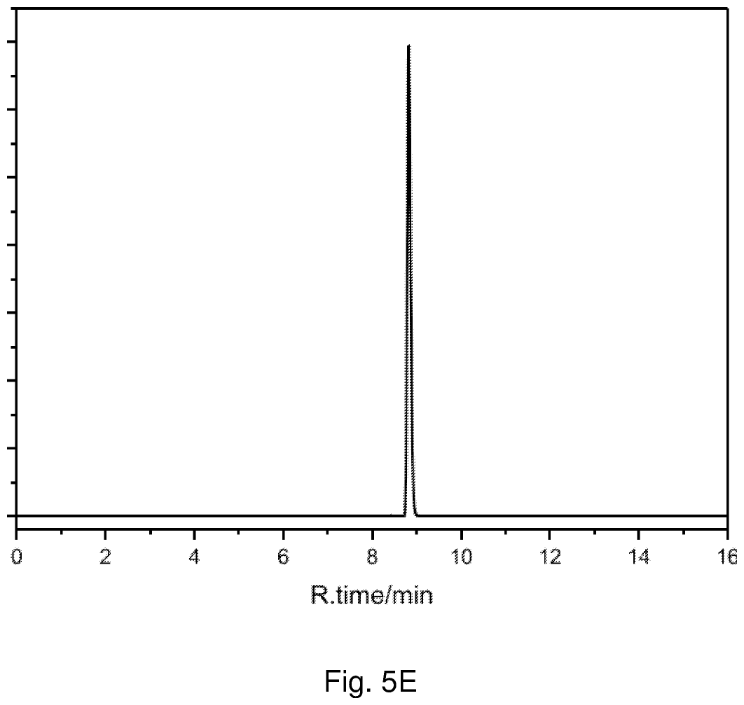

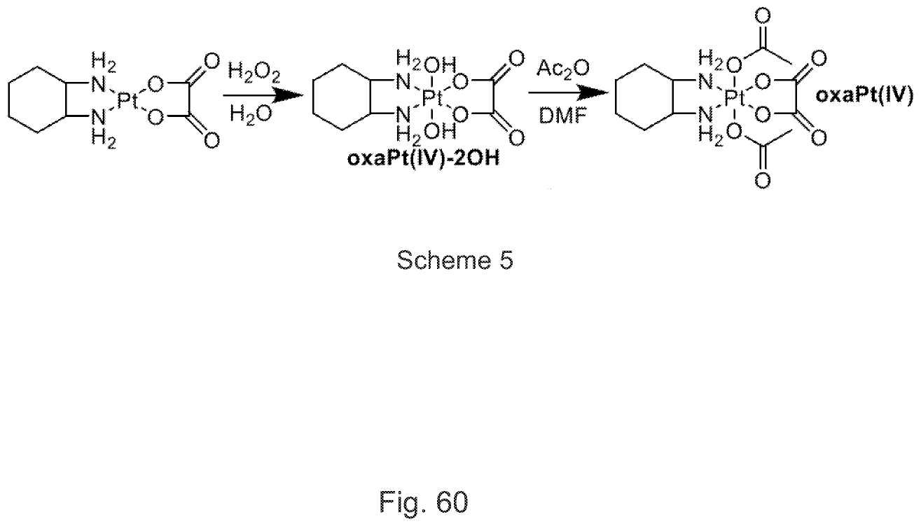

The synthesis of oxaPt(IV) is summarized in FIG. 60 (Scheme 5). Complex oxaPt(IV)-2OH was synthesized according to reported method. 400 mg oxaPt(IV)-2OH was suspended in 3 mL acetic anhydride. The reaction mixture was stirred for 6 h at room temperature. Et₂O was then added. The precipitate was collected by centrifuge, further washed with EtOH and Et₂O, and finally dried in vacuum. ¹H NMR (600 MHZ, DMSO-d6) δ 8.33 (d, J=40.2 Hz, 4H), 2.56 (s, 2H), 2.10 (d, J=12.4 Hz, 2H), 1.95 (s, 6H), 1.54-1.46 (m, 2H), 1.42 (s, 2H), 1.21-1.10 (m, 2H) (FIG. 5A). ¹³C NMR (151 MHz, DMSO-d6) δ 178.93, 163.86, 61.47, 31.34, 23.95, 23.44 (FIG. 5B). ¹⁹⁵Pt NMR (129 MHz, DMSO-d6) δ 1615.06 (FIG. 5C). [M+H]⁺ calculated for C₁₂H₂₁N₂O₈Pt: 510.0941, found: 510.0937 (FIG. 5D). HCLP chromatogram of the as-obtained oxaPt(IV) shows a single peak, suggesting the high purity of the complex (FIG. 5E).

### Example 1F

Synthesis of transPt(IV) (Pt(IV) Complex of Formula (IIf))

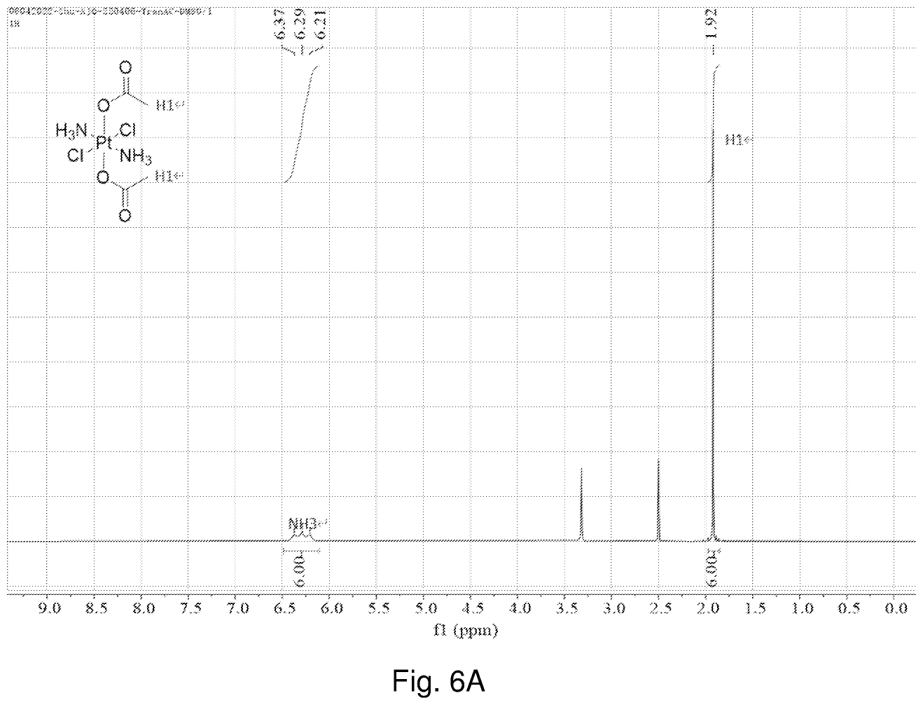

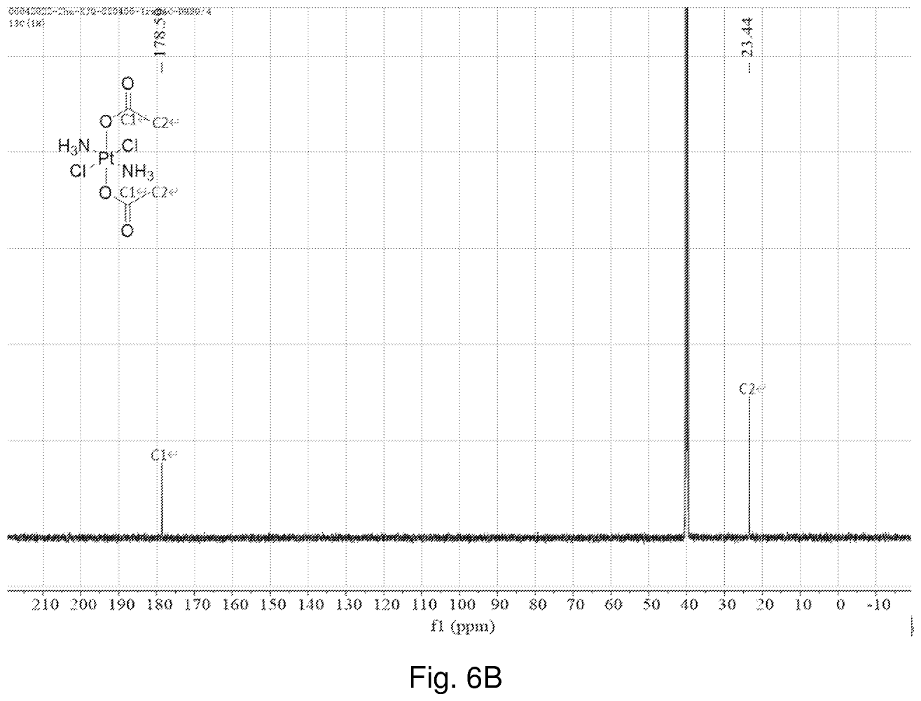

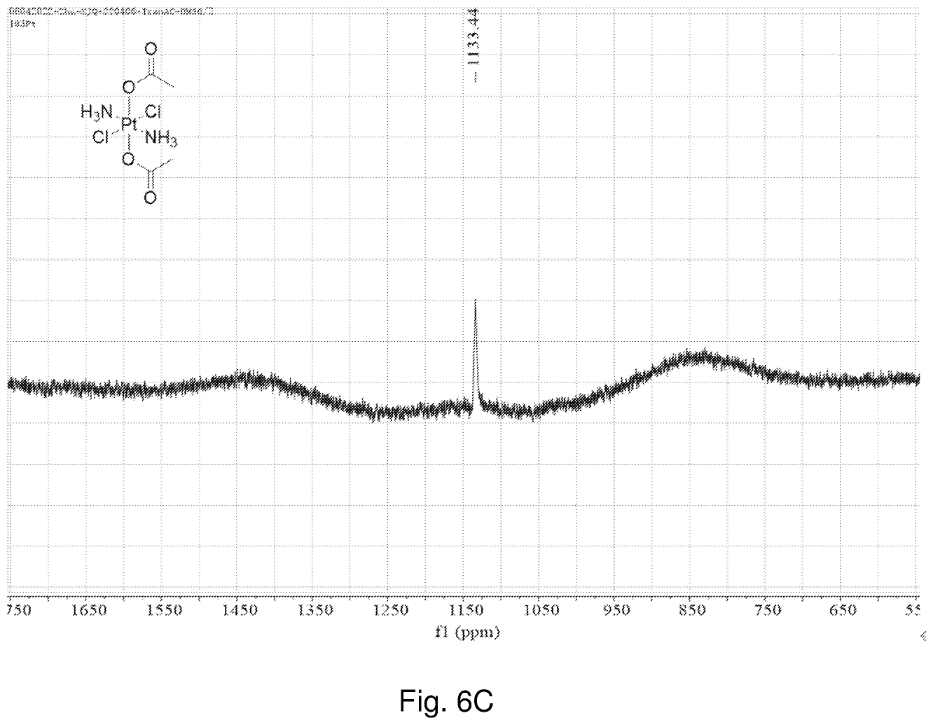

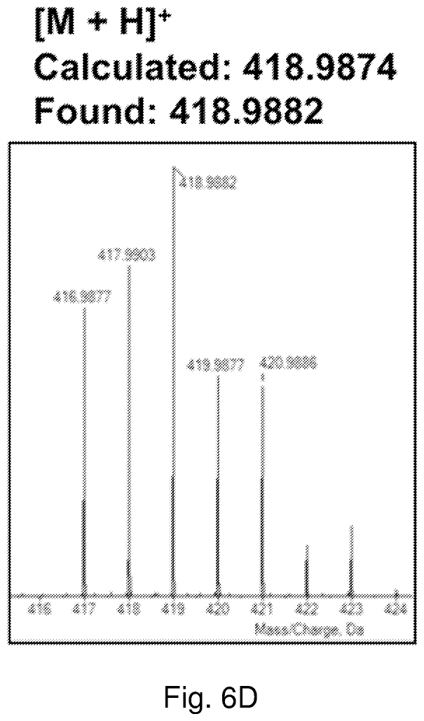

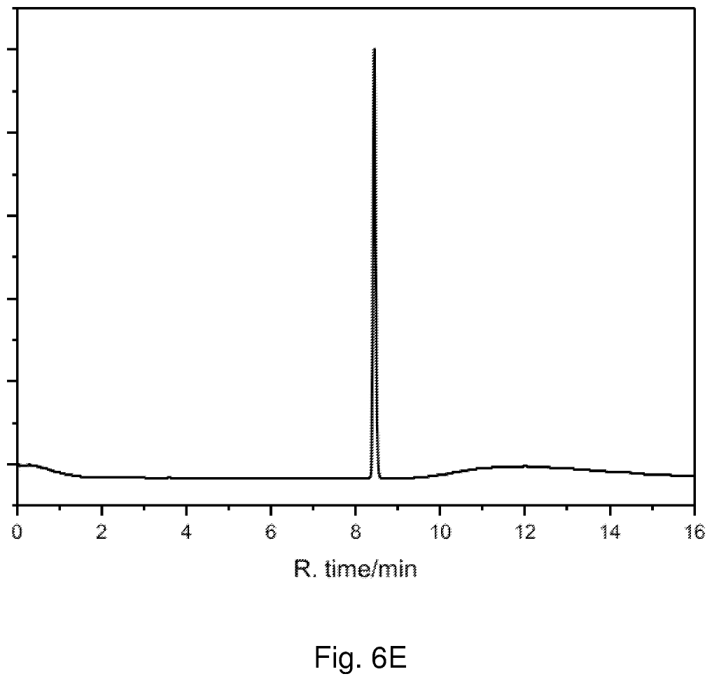

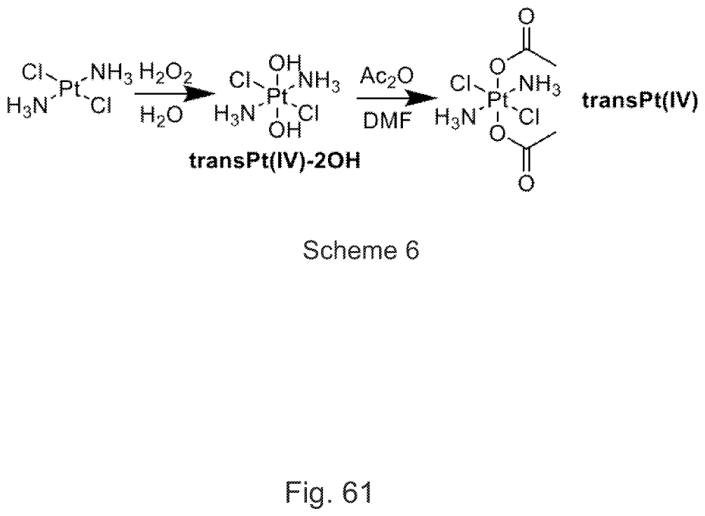

The synthesis of transPt(IV) is summarized in FIG. 61 (Scheme 6). 150 mg transplatin was suspended in 8 mL H₂O. 1 mL of 30% H₂O₂ was added, and the reaction mixture was stirred for 24 h at room temperature. The solvent was removed with vacuum. The solid was washed with methanol and Et₂O and dried. The solid was suspended in 1 mL acetic anhydride and stirred for 3 days at room temperature. The desired product was directly purified by HPLC. ¹H NMR (600 MHZ, DMSO-d6) δ 6.49-6.11 (m, 6H), 1.92 (s, 6H) (FIG. 6A). ¹³C NMR (151 MHz, DMSO-d6) δ 178.59, 23.44 (FIG. 6B). ¹⁹⁵Pt NMR (129 MHz, DMSO-d6) δ 1133.44 (FIG. 6C). ESI-HRMS (positive mode) m/z: [M+H]⁺ calculated for C₄H₁₃Cl₂N₂O₄Pt: 418.9874, found: 418.9882 (FIG. 6D). HCLP chromatogram of the as-obtained transPt(IV) shows a single peak, suggesting the high purity of the complex (FIG. 6E).

### Example 1G

### Synthesis of Alkyne-Pt(IV) (Pt(IV) Complex of Formula (IV))

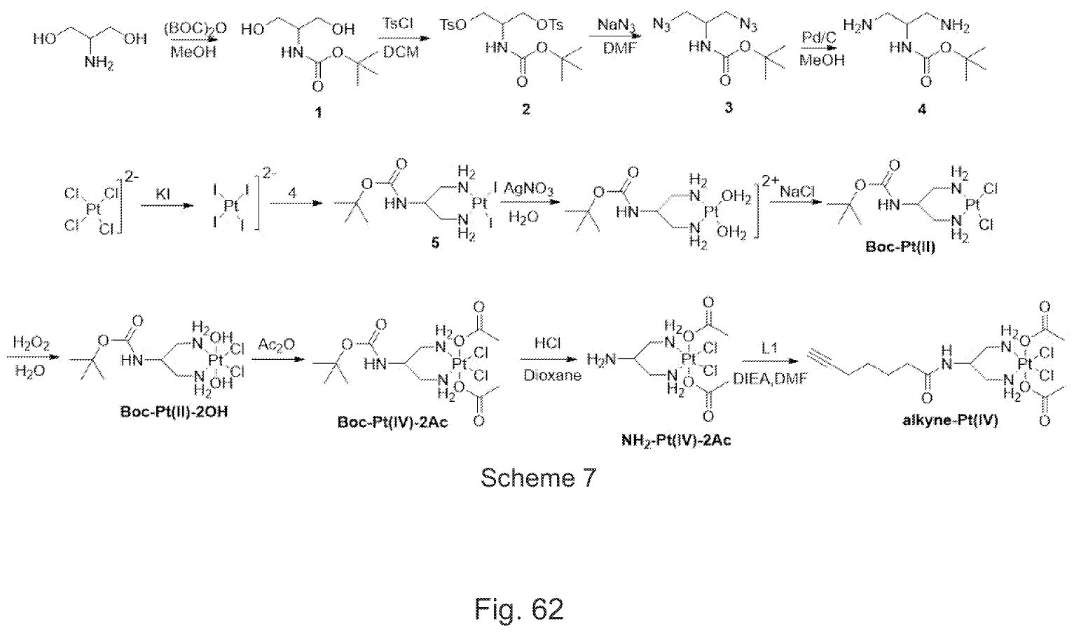

The synthesis of alkyne-Pt(IV) is summarized in FIG. 62 (Scheme 7). As shown, the synthesis of alkyne-Pt(IV) involves the reaction of NH₂—Pt(IV)-2Ac and compound L1, which are respectively prepared as follows:

Synthesis of compound L1

6-Heptynoic acid (126 mg, 1 mmol) was dissolved in 2 mL DCM, followed by addition of NHS (138 mg, 1.2 mmol) and EDCI (287.5 mg, 1.5 mmol) was then added. The resulting reaction mixture was stirred at room temperature for 8 h. Upon solvent evaporation, the residue was dissolved in EA, washed with brine, followed by solvent evaporation via vacuum to yield L1 as pale yellow liquid, which solidified upon standing. ¹H NMR (300 MHz, Chloroform-d) δ 2.87 (d, J=2.5 Hz, 4H), 2.68 (t, J=7.4 Hz, 2H), 2.28 (td, J=6.9, 2.7 Hz, 2H), 2.00 (t, J=2.7 Hz, 1H), 1.91 (dt, J=15.3, 7.4 Hz, 2H), 1.74-1.62 (m, 3H).

### Synthesis of NH₂—Pt(IV)-2Ac

As shown in Scheme 7, the synthesis of NH₂—Pt(IV)-2Ac commences with the synthesis of compound 1. Specifically, 2-aminopropane-1,3-diol (900 mg, 10 mmol) and TEA (1.38 mL, 10 mmol) was dissolved in 5 mL ethanol. Then Di-tert-butyl dicarbonate (2.5 mL, 10 mmol) in methanol (3 mL) was added dropwise. The reaction mixture was stirred at room temperature for 5 h, the solvent was removed under reduced pressure. The crude product was washed with brine and extracted with EA to yield compound 1 as a white powder.

Compound 1 is then converted to compound 2 as follows. To a solution of compound 1 (956 mg, 5 mmol) was added TEA (2.1 mL, 15 mmol), and then added tosyl chloride (2.85 g, 15 mmol) slowly. The reaction mixture was stirred at room temperature for 2 h. The mixture was extracted DCM, washed with 1 M HCl and saturated NaHCO₃. The organic phase was dried and purified with column (PE (petroleum ether):EA (ethyl acetate)=4:1) to yield compound 2 as white powder. ¹H NMR (600 MHZ, Chloroform-d) δ 7.78 (d, J=8.3 Hz, 4H), 7.38 (d, J=8.1 Hz, 4H), 4.82 (d, J=7.7 Hz, 1H), 4.09-4.02 (m, 4H), 2.49 (s, 6H), 1.42 (s, 9H).

Then compound 2 is converted to compound 3, which is subsequently converted to compound 4 as follows. To a solution of compound 2 (1.6 g, 3.2 mmol) in 5 mL DMF was added NaN₃ (1.66 g, 25.6 mmol). The reaction mixture was heated overnight at 60° C. Saturated NaHCO₃ was then added to quench the reaction. Upon extraction with DCM, the organic phase was washed with brine and dried over Na₂SO₄. The solvent was evaporated under vacuum and purified with column (PE:EA=10:1) to give compound 3 as yellow oil. ¹H NMR (600 MHZ, Chloroform-d) δ 3.88 (d, J=7.5 Hz, 1H), 3.48 (ddd, J=65.9, 12.3, 5.4 Hz, 4H), 1.45 (s, 9H).

After that, to a suspension containing compound 3 (280 mg, 1.16 mmol) and Pd—C (10%) in a 50 mL round-bottom flask was connected to a hydrogen balloon and deoxygenated methanol was carefully added. The resulting suspension was stirred at room temperature for 24 h and after this time, the reaction was stopped by filtering the mixture through celite. The solvent was evaporated under reduced pressure to yield compound 4 as colorless oil. ¹H NMR (600 MHZ, Methanol-d4) δ 3.48 (ddd, J=7.3, 5.4, 2.1 Hz, 1H), 3.31 (p, J=1.6 Hz, 4H), 2.71 (dd, J=13.2, 5.3 Hz, 2H), 2.59 (dd, J=13.2, 7.4 Hz, 2H), 1.45 (s, 9H).

The synthesis of NH₂—Pt(IV)-2Ac then proceed to the step of synthesis of compound 5. Specifically, potassium platinochloride (208 mg, 0.5 mmol) was dissolved in 2 mL water and allowed to stirred for 10 min. Potassium iodide was dissolved in 2 mL water and added to the mixture, resulting in a violet solution which was further stirred for 15 min. compound 4 (94.5 mg, 0.5 mmol) in 2 water was added to the mixture dropwise, resulting in yellow precipitate, which was further stirred overnight at room temperature in the dark. Then the precipitate was collected by centrifugation, and washed successively with water, ethanol and Et₂O to yield compound 5 as brown yellow precipitate.

Compound 5 is then converted, in order, to Boc-Pt(II), Boc-Pt(IV)-2OH, Boc-Pt(IV)-2Ac, and finally NH₂—Pt(IV)-2Ac. Specifically, compound 5 was suspended into water, and silver nitrate (153 mg, 0.9 mmol) dissolved in water was added to the slurry and stirred overnight at room temperature. The silver iodide precipitate was removed by centrifugation. And 2 mL saturated sodium chloride solution was added to the filtrate, and stirred overnight at room temperature in the dark. Then the precipitate was collected and washed with water, ethanol and Et₂O to yield Boc-Pt(II) as white powder. ¹H NMR (600 MHz, DMF-d7) δ 7.06-6.99 (m, 1H), 5.24 (s, 4H), 3.82 (d, J=8.7 Hz, 1H), 2.84-2.78 (m, 2H), 2.70 (dp, J=12.6, 4.0 Hz, 2H), 1.40 (s, 9H).

Boc-Pt(II) (60 mg, 0.132 mmol) was then suspended in 500 μL water, followed by addition of 50 μL 30% H₂O₂ in ice bath and stirred overnight at room temperature in the dark. The solid was washed with EtOH and Et₂O, and dried in vacuum to yield Boc-Pt(IV)-2OH as white solid. ¹H NMR (300 MHz, DMSO-d6) δ 7.20 (d, J=7.8 Hz, 1H), 6.50 (s, 4H), 2.34 (s, 4H), 1.39 (s, 9H).

After that, suspending the Boc-Pt(IV)-2OH in acetic anhydride and stirred overnight at room temperature. The solvent was evaporated under vacuum and precipitate with Et₂O. Collected the solid, washed with Et₂O to yield Boc-Pt(IV)-2Ac as white powder. ¹H NMR (400 MHZ, DMSO-d6) δ 8.23 (s, 2H), 7.63 (s, 2H), 6.98 (d, J=6.8 Hz, 1H), 3.72 (d, J=8.7 Hz, 1H), 2.47-2.24 (m, 4H), 1.95 (d, J=3.0 Hz, 6H), 1.39 (t, J=2.2 Hz, 9H).

Finally, to a centrifugation tube charged with 10 mg Boc-Pt(IV)-2Ac chilled on ice was added 50 μL 4 M HCl in dioxane. The mixture was stirred for 2 h at room temperature. Then the solvent was evaporated directly to yield crude product NH₂—Pt(IV)-2Ac as yellow solid. ¹H NMR (300 MHz, DMSO-d6) δ 8.50 (s, 2H), 8.21 (s, 3H), 7.75-7.36 (m, 2H), 2.58 (d, J=14.6 Hz, 4H), 1.96 (d, J=8.9 Hz, 6H). ESI-HRMS (positive mode) m/z: [M+H]⁺ calcd. for C₇H₁₇Cl₂N₃O₄Pt: 474.0297, found: 474.0304.

### Synthesis of Alkyne-Pt(IV)

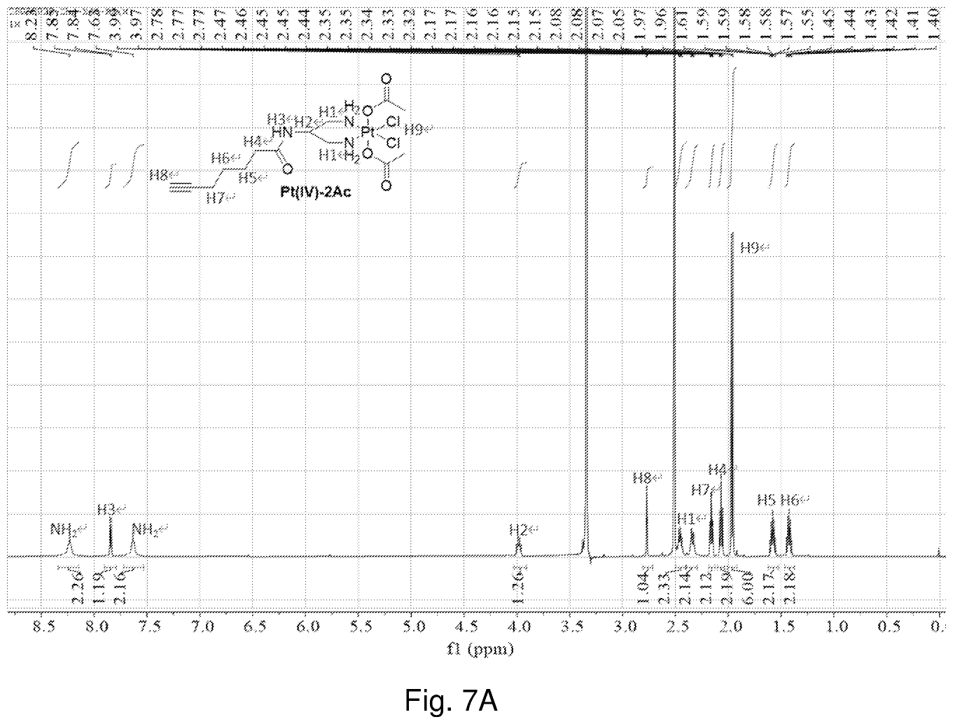

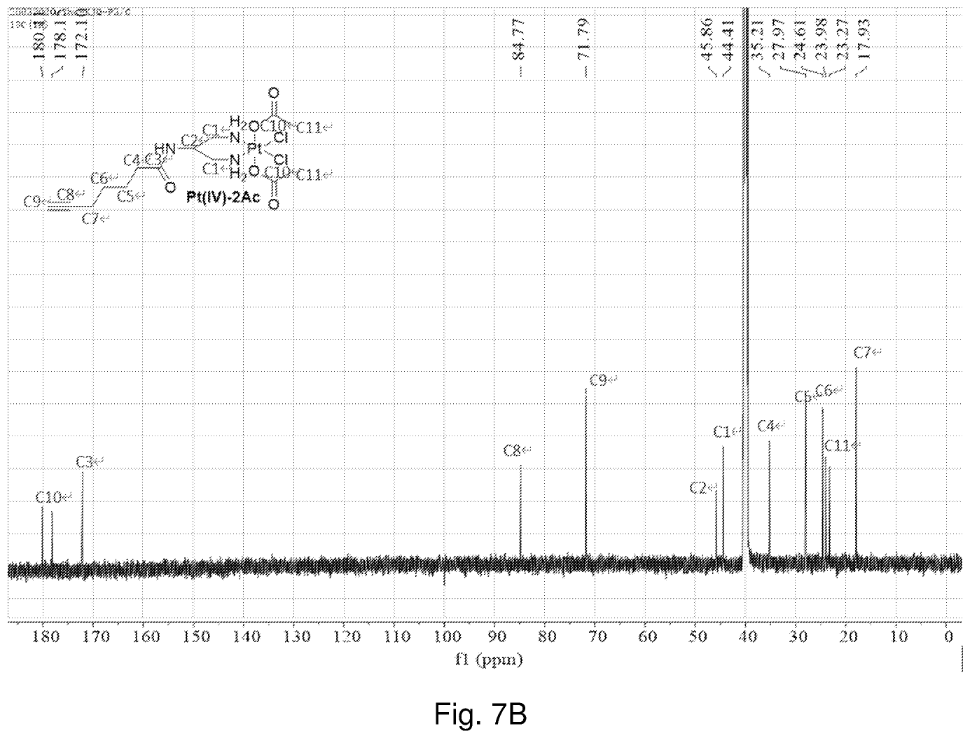

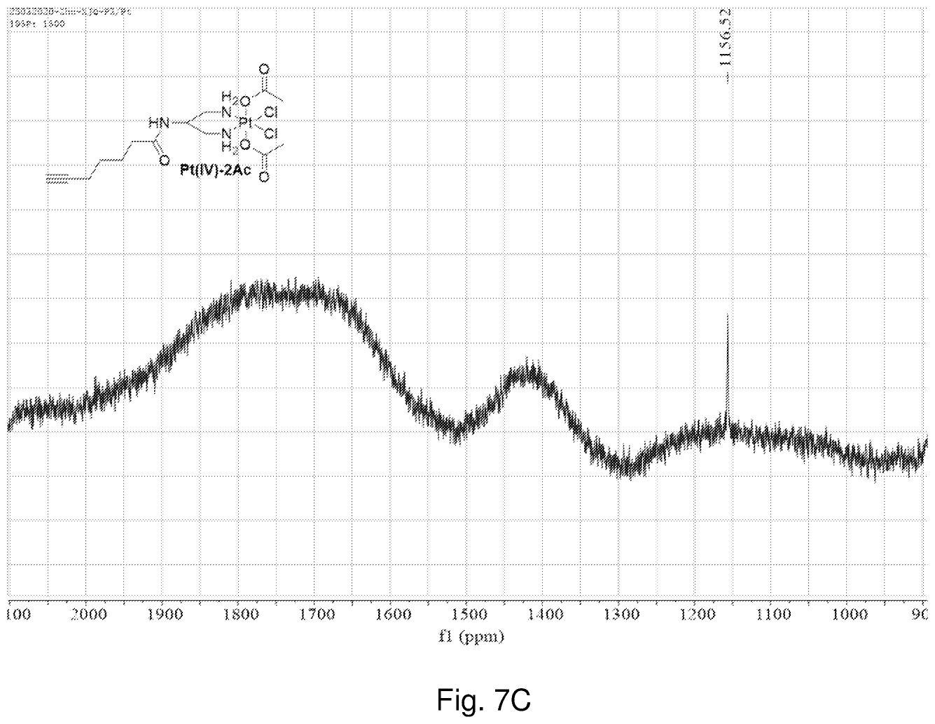

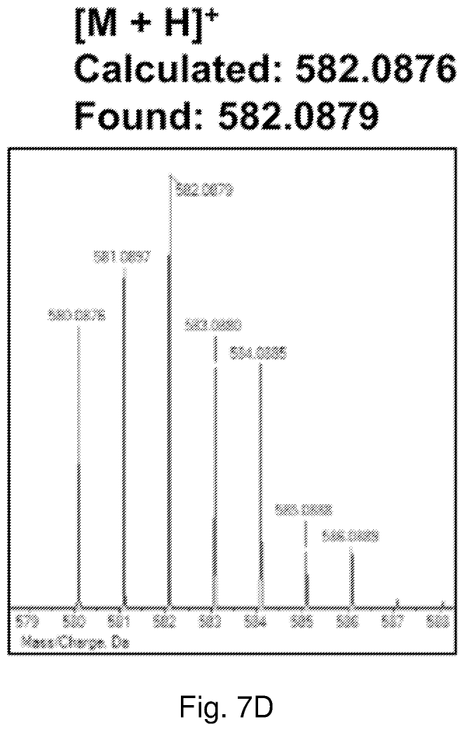

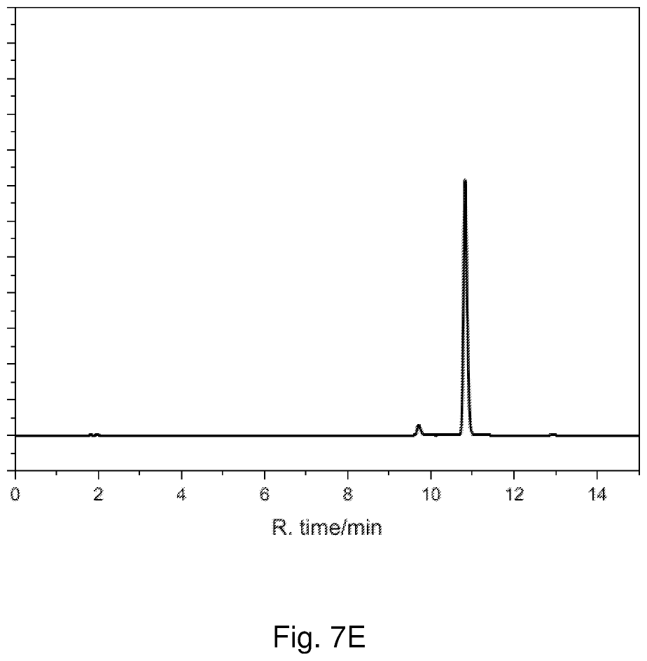

To a solution of NH₂—Pt(IV)-2Ac (5 mg, 0.0105 mmol) in 50 μL DMF was added L1 (2.8 mg, 0.0126 mmol) and TEA (7.5 μL, 0.0432 mmol). The mixture was stirred at room temperature overnight. The solvent was evaporated by vacuum and purified by preparative HPLC to yield alkyne-Pt(IV) as white solid. ¹H NMR (600 MHZ, DMSO-d6) δ 8.27 (s, 2H), 7.99 (d, J=7.1 Hz, 1H), 7.58 (s, 2H), 3.99-3.91 (m, 1H), 2.84 (t, J=2.7 Hz, 1H), 2.44 (dq, J=9.2, 5.2, 4.6 Hz, 2H), 2.30 (dd, J=13.2, 3.4 Hz, 2H), 2.22 (s, 2H), 2.02 (td, J=7.4, 2.7 Hz, 2H), 1.95 (d, J=4.3 Hz, 6H), 1.66 (t, J=7.4 Hz, 2H) (FIG. 7A). ¹³C NMR (151 MHz, DMSO-d6) δ 180.11, 178.15, 172.10, 84.77, 71.79, 45.86, 44.41, 35.21, 27.97, 24.61, 23.98, 23.27, 17.93 (FIG. 7B). ¹⁹⁵Pt NMR (129 MHz, DMSO-d6): δ 1156.52 (s) (FIG. 7C). ESI-HRMS (positive mode) m/z: [M+H]⁺ calcd. for C₁₄H₂₅Cl₂N3O5Pt: 582.0876, found: 582.0879 (FIG. 7D). HCLP chromatogram of the as-obtained alkyne-Pt(IV) shows a single peak, suggesting the high purity of the complex (FIG. 7E).

### Example 2A

### Synthesis of Comparison Compound PC-1

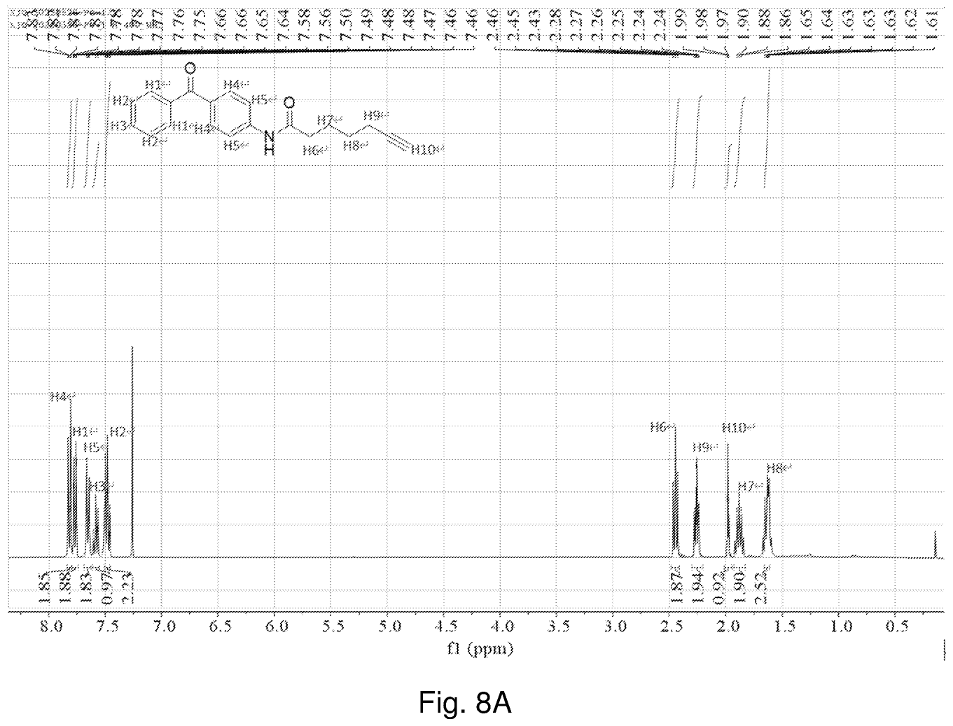

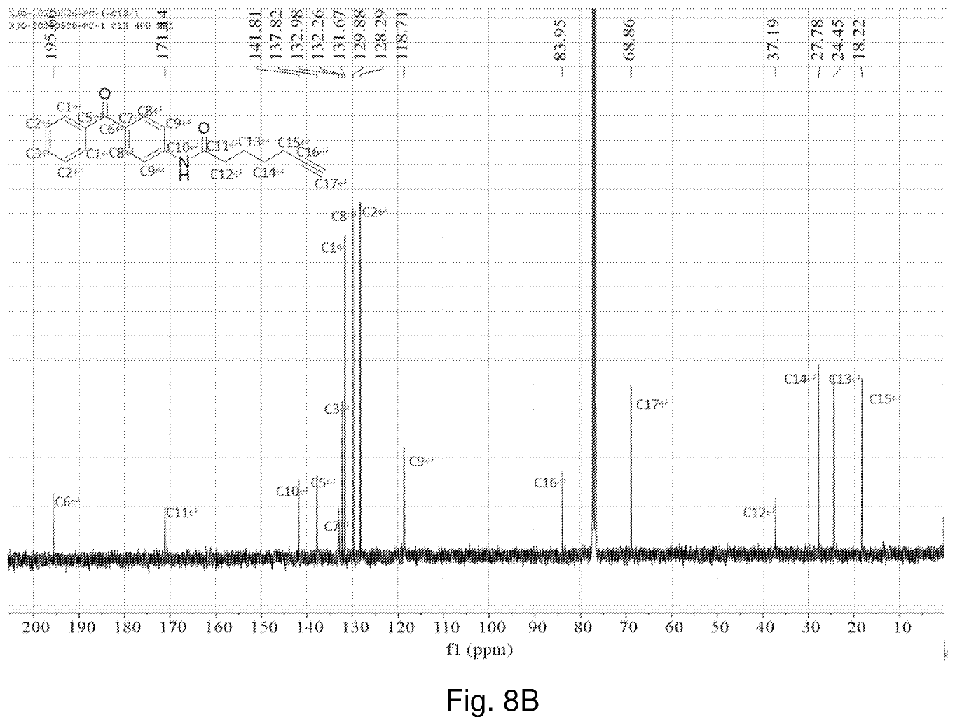

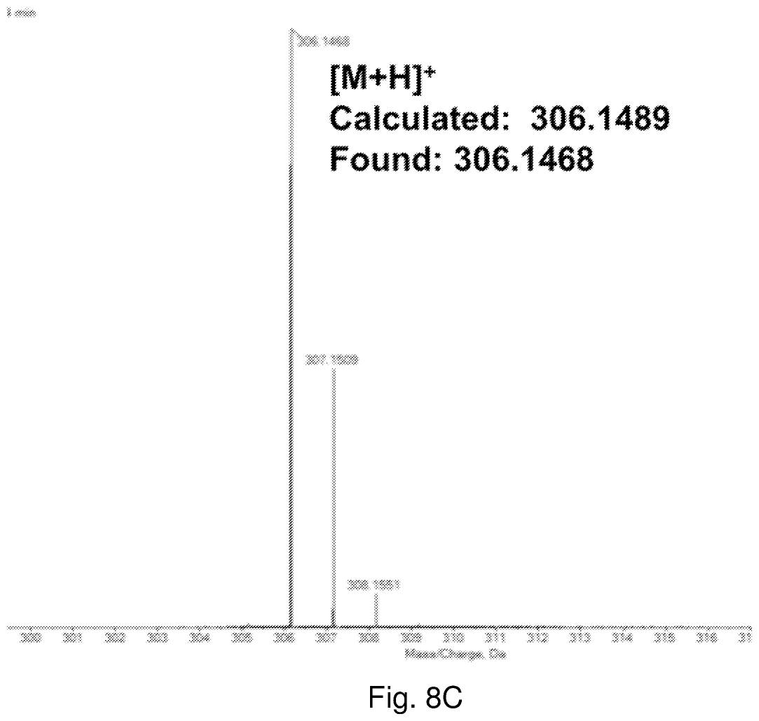

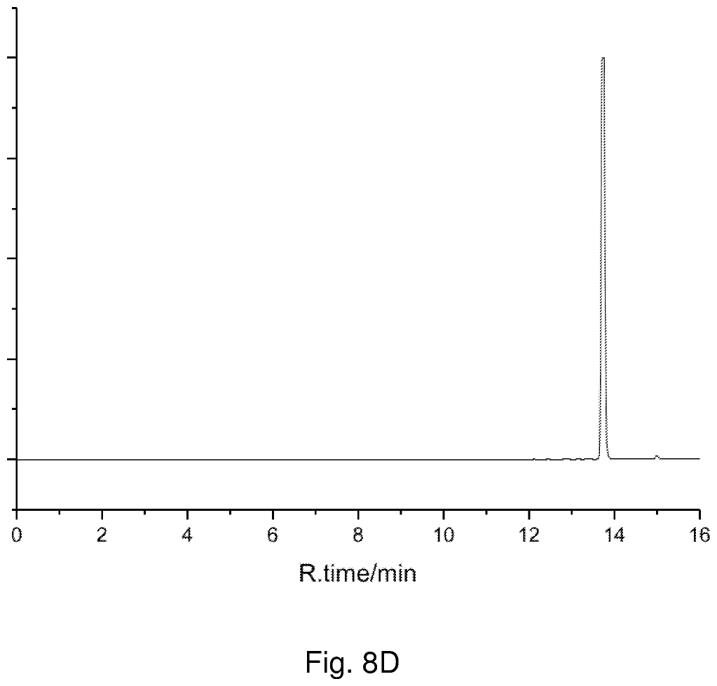

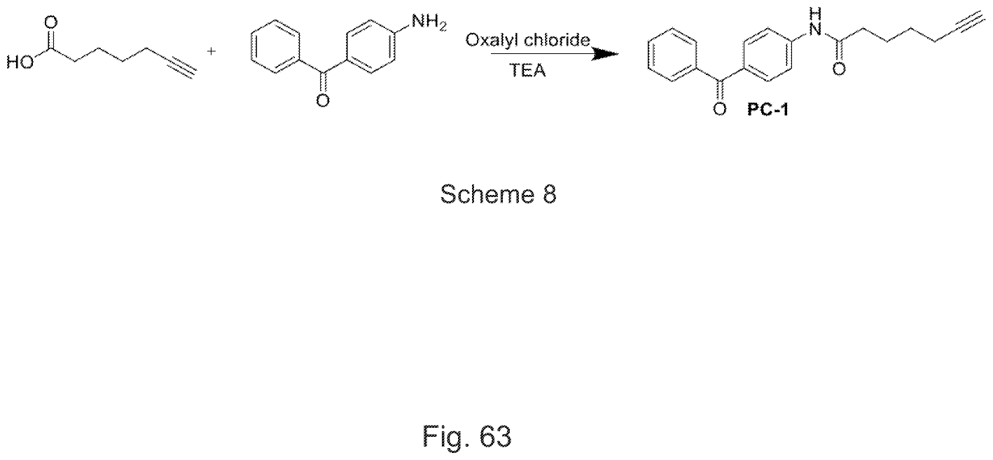

The synthesis of PC-1 is summarized in FIG. 63 (Scheme 8). To a solution of 225 mg (1 eq.) 6-heptynoic acid in 12 mL DCM was added 40 μL DMF. The mixture was cooled in an ice bath, and oxalyl chloride 304.8 mg (1.3 eq.) was added. The mixture was stirred for 1 h. 4-Aminobenzophenone (352 mg, 1 eq.) and TEA (0.4 mL) were added. The mixture was stirred at room temperature for 5 h, and the reaction was quenched by water. The organic phase was washed with brine, and the product was purified by column chromatography (PE:EA=3:1). ¹H NMR (400 MHZ, Chloroform-d) δ 7.84-7.80 (m, 2H), 7.80-7.74 (m, 2H), 7.69-7.62 (m, 2H), 7.61-7.55 (m, 1H), 7.50-7.46 (m, 2H), 2.45 (t, J=7.4 Hz, 2H), 2.26 (td, J=7.0, 2.6 Hz, 2H), 1.98 (t, J=2.6 Hz, 1H), 1.93-1.83 (m, 2H), 1.65-1.61 (m, 2H) (FIG. 8A). ¹³C NMR (101 MHZ, Chloroform-d) δ 171.14, 141.81, 137.82, 132.26, 131.67, 129.88, 128.29, 118.71, 83.95, 68.86, 37.19, 27.78, 24.45, 18.22 (FIG. 8B). ESI-HRMS (positive mode) m/z: [M+H]⁺ calculated for C₂₀H₂₀NO₂: 306.1489, found: 306.1468 (FIG. 8C). HCLP chromatogram of the as-obtained PC-1 shows a single peak, suggesting the high purity of the compound (FIG. 8D).

### Example 2B

### Synthesis of Comparison Compound PC-2

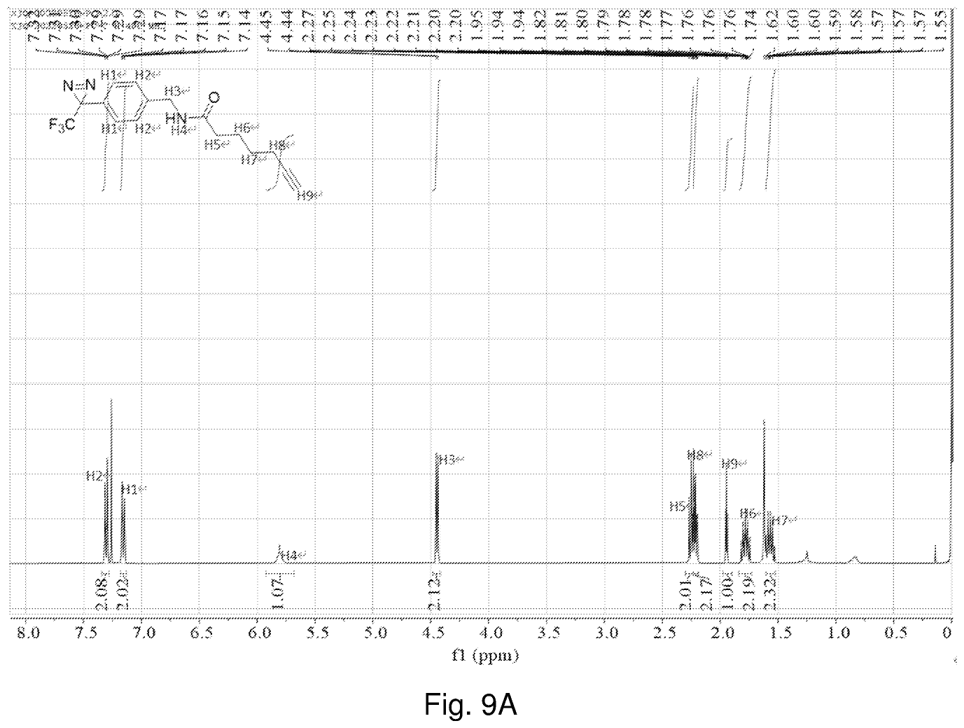

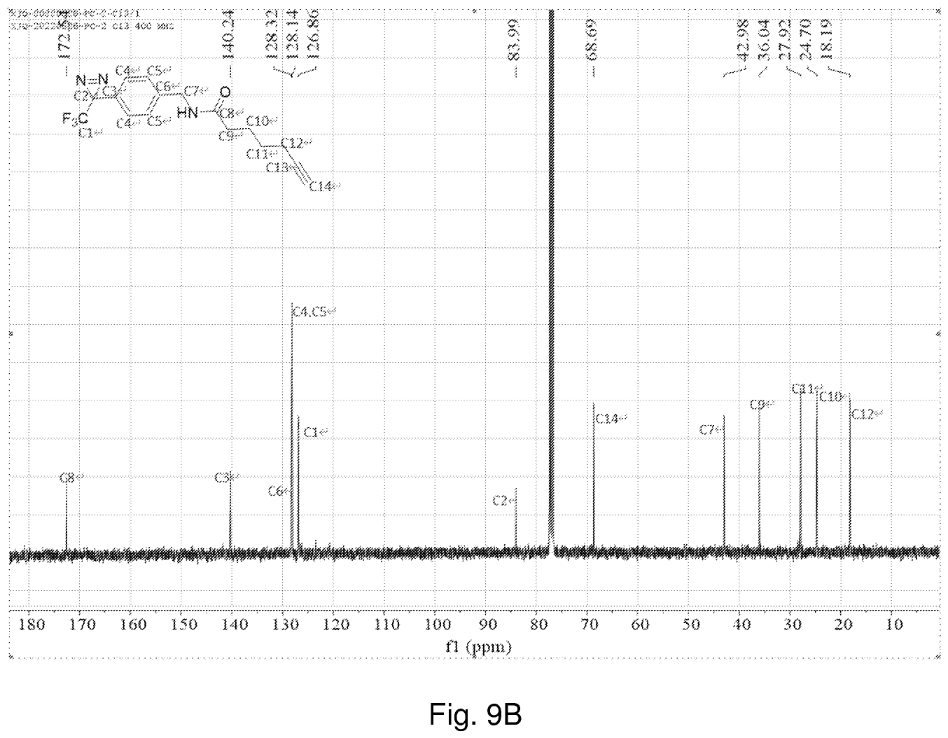

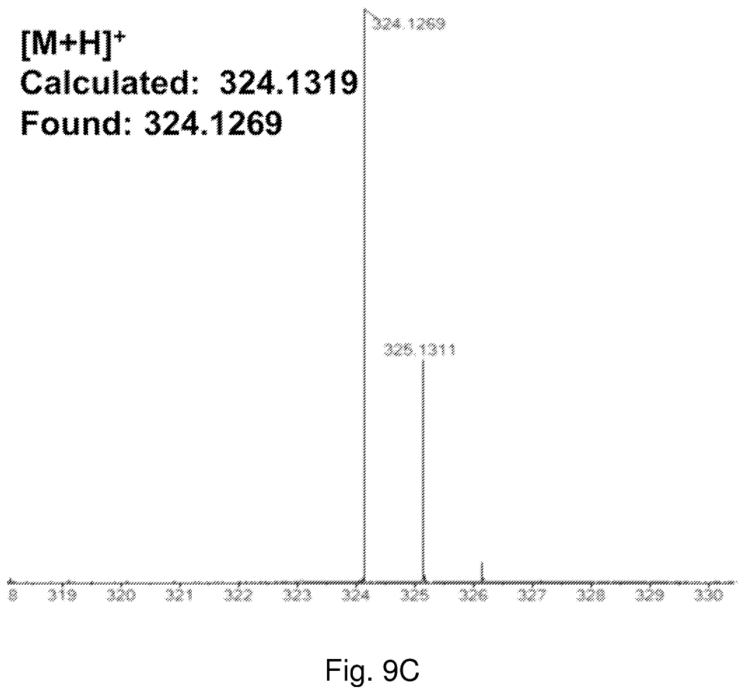

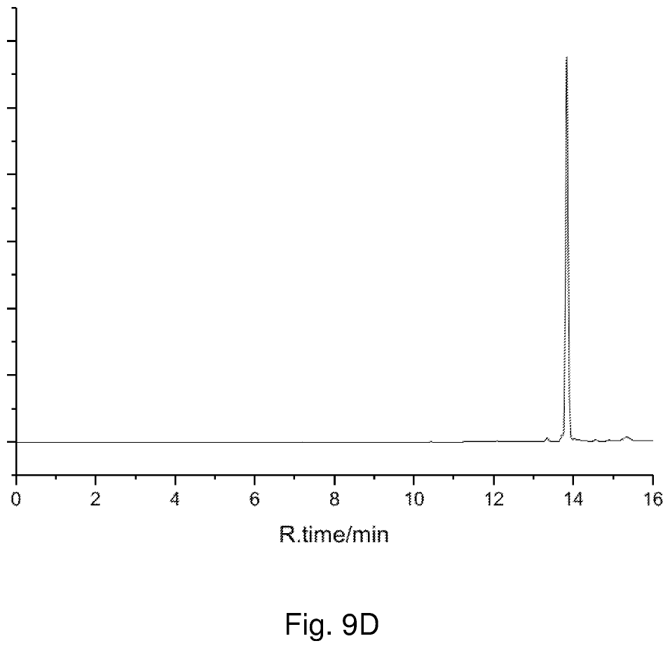

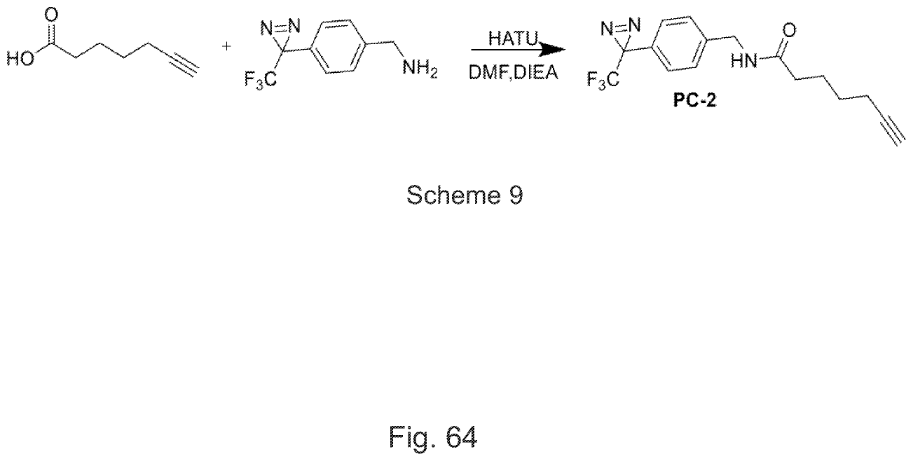

The synthesis of PC-2 is summarized in FIG. 64 (Scheme 9). To a solution of 6-heptynoic acid (63 mg, 1 eq.) in DMF was added HATU (228.1 mg, 1.2 eq.), 4-[3-(Trifluoromethyl)-3H-diazirin-3-yl]benzylamine hydrochloride (113.2 mg, 0.9 eq.) and DIEA (77.5 mg, 1.2 eq). The solution was stirred at room temperature overnight. The reaction was quenched by water. The organic phase was extracted with EA and further purified by column chromatography (PE:EA=3:1). ¹H NMR (400 MHZ, Chloroform-d) δ 7.34-7.28 (m, 2H), 7.18-7.13 (m, 2H), 5.81 (s, 1H), 4.45 (d, J=5.9 Hz, 2H), 2.30-2.23 (m, 2H), 2.23-2.19 (m, 2H), 1.94 (t, J=2.7 Hz, 1H), 1.84-1.73 (m, 2H), 1.60-1.52 (m, 2H) (FIG. 9A). ¹³C NMR (101 MHZ, chloroform-d) δ 172.54, 140.24, 128.32, 128.14, 126.86, 83.99, 68.69, 42.98, 36.04, 27.92, 24.70, 18.19 (FIG. 9B). ESI-HRMS (positive mode) m/z: [M+H]⁺ calculated for C₁₆H₁₇F₃N₃O: 324.1319, found: 306.1269 (FIG. 9C). HCLP chromatogram of the as-obtained PC-2 shows a single peak, suggesting the high purity of the compound (FIG. 9D).

### Example 2C

### Synthesis of Control Complex Alkyne-Pt(II)

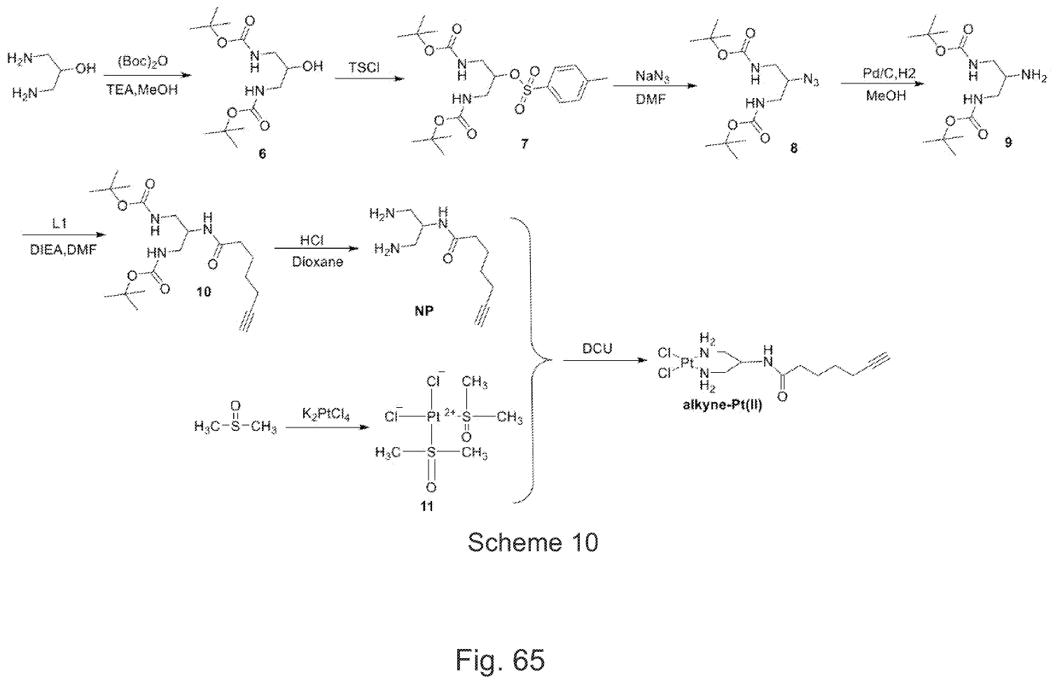

The synthesis of alkyne-Pt(II) is summarized in FIG. 65 (Scheme 10). As illustrated in FIG. 65, the formation of alkyne-Pt(II) involves the reaction of compound NP and compound 11, which are respectively, prepared as follows.

### Synthesis of Compound NP

As shown, the synthesis of compound NP commences at the step of synthesizing the compound 6. Specifically, a solution of 1,3-diaminopropan-2-ol (900.1 mg, 10 mmol) and TEA (1.38 mL, 10 mmol) in methanol (3 mL) was heated to 45° C. Then di-tert-butyl pyrocarbonate (20 mL, 80 mmol) in methanol (3 mL) was added dropwise. The reaction mixture was stirred at 45° C. for 30 min. After additional stirring at room temperature for 2 h, the solvent was removed under reduced pressure. The crude product was extracted with EA to yield compound 6 as white powder. ¹H NMR (400 MHz, Chloroform-d) δ 5.48 (t, J=6.2 Hz, 2H), 3.67 (q, J=5.3 Hz, 1H), 3.10 (t, J=5.7 Hz, 4H), 1.36 (s, 18H).

Compound 6 is then converted, in order, to compounds 7 to 10 as follows. To a solution of compound 6 (2.62 g, 9 mmol) in DCM was added TEA (1.86 mL, 13.4 mmol) and tosyl chloride (2.04 g, 10.74 mmol) slowly. The mixture was stirred for 2 h at room temperature. The mixture was extracted DCM, washed with 1 M HCl and saturated NaHCO₃. The organic phase was dried and purified with column (PE:EA=5:1) to yield compound 7 as white powder. ¹H NMR (400 MHZ, Chloroform-d) δ 7.81 (d, J=8.0 Hz, 2H), 7.35 (d, J=8.1 Hz, 2H), 5.01 (s, 2H), 4.54 (s, 1H), 3.48-3.11 (m, 4H), 2.45 (s, 3H), 1.41 (s, 18H).

Then, to a solution of compound 7 (2.06 g, 4.6 mmol) in 15 mL dry DMF was added NaN3 (1.2 g, 18.4 mmol). The mixture was heated overnight at 60° C. Saturated NaHCO3 was then added to quench the reaction. Upon extraction with DCM, the organic phase was washed with brine and dried over Na₂SO₄. The solvent was evaporated under vacuum and purified with column (PE:EA=10:1) to give compound 8 as white powder. ¹H NMR (400 MHZ, Chloroform-d) δ 7.81 (d, J=8.0 Hz, 2H), 7.35 (d, J=8.1 Hz, 2H), 5.01 (s, 2H), 4.54 (s, 1H), 3.48-3.11 (m, 4H), 2.45 (s, 3H), 1.41 (s, 18H).

After that, to a solution of compound 8 (1.15 g, 3.65 mmol) in 5 mL MeOH was added Pd—C (10%, 300 mg) and the mixture was hydrogenated with balloon for 24 h. The reaction mixture was filtered over celite and evaporated under reduced pressure and purified by column (MeOH:DCM=30:1) to yield the desired product (compound 9 as clear oil, which solidified upon standing. ¹H NMR (400 MHZ, Chloroform-d) δ 5.07 (s, 2H), 3.64 (p, J=5.5 Hz, 1H), 3.36 (s, 2H), 3.14 (dd, J=13.0, 6.7 Hz, 2H), 1.44 (s, 18H).

Compound 9 is further converted to compound 10 by reacting with compound L1. Specifically, to a solution of EDCI (75.8 mg, 0.394 mmol) in anhydrous DCM was added L1 (50 mg, 0.329 mmol) while stirring in an ice bath. Compound 9 (114 mg, 0.394 mmol) in dry DCM was added dropwise. The reaction was then stirred for additionally 2 days at room temperature. The reaction was then quenched with H₂O while stirring for 15 min. The mixture was then filtered and the filtrate was washed with HCl (1M) and saturated NaHCO₃, and finally purified with column (PE:EA=3:1) to yield compound 10 as white powder.

Finally, to a flask charged with compound 10 (60 mg, 0.14 mmol) chilled on ice was added 4 M HCl in dioxane. The mixture was stirred for 2 h at room temperature. Then the solvent was evaporated directly to yield product NP as yellow solid. ¹H NMR (600 MHz, DMSO-d6) δ 8.23 (t, J=6.5 Hz, 7H), 4.20 (dq, J=8.2, 4.0, 3.4 Hz, 1H), 3.07-2.97 (m, 2H), 2.91 (ddd, J=13.3, 7.9, 5.6 Hz, 2H), 2.76 (t, J=2.7 Hz, 1H), 2.19 (t, J=7.6 Hz, 2H), 2.16 (td, J=7.1, 2.7 Hz, 2H), 1.64-1.57 (m, 2H), 1.48-1.41 (m, 2H). ¹³C NMR (151 MHZ, DMSO-d6) δ 173.53, 84.89, 71.74, 45.79, 35.22, 28.1, 24.18, 18.06. ESI-HRMS (positive mode) m/z: [M+H]⁺ calcd. for C₁₀H₁₉N₃O: 198.1061, found: 198.1596.

### Synthesis of Compound 11

Adding DMSO (106 μL, 1.5 mmol) to an aqueous solution of potassium platinochloride (310 mg, 0.75 mmol) in 10 mL of water and allowing the solution to be stirred at room temperature for 2 h. The precipitate was filtered, washed with water, ethanol and ether, and dried in vacuo overnight. ¹H NMR (300 MHz, DMF-d7) δ 3.85 (s, 12H).

Synthesis of alkyne-Pt(II)

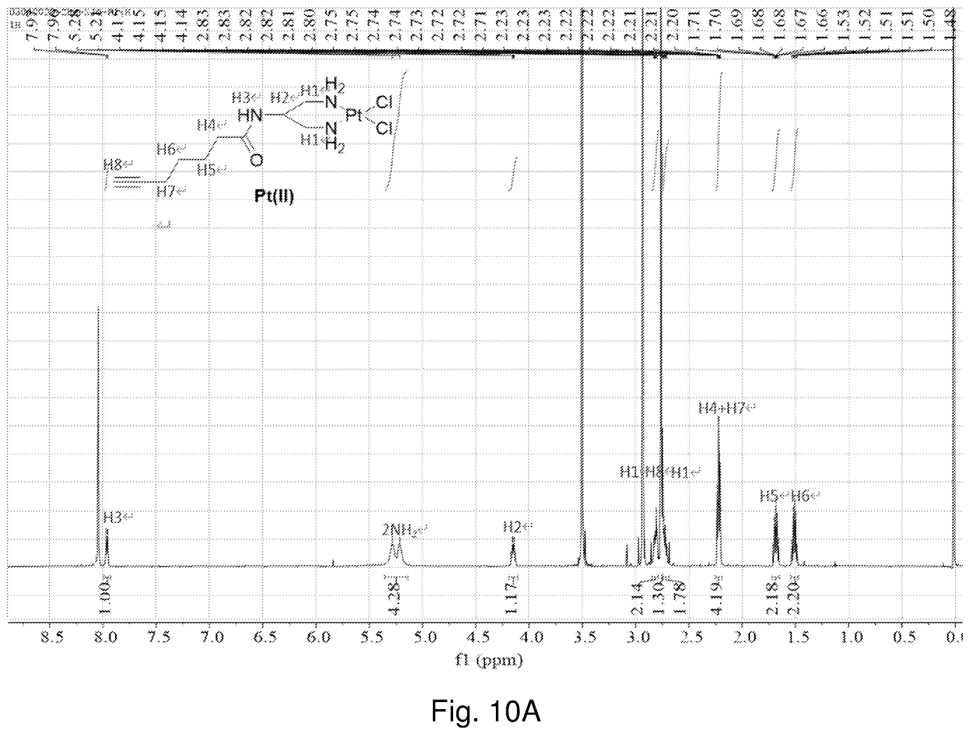

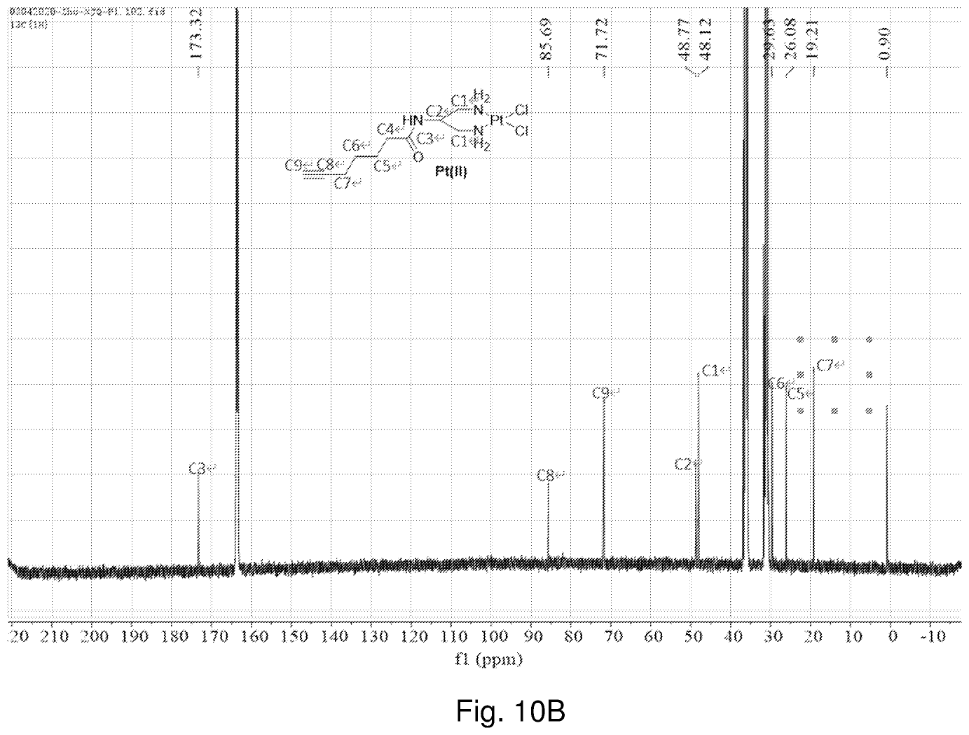

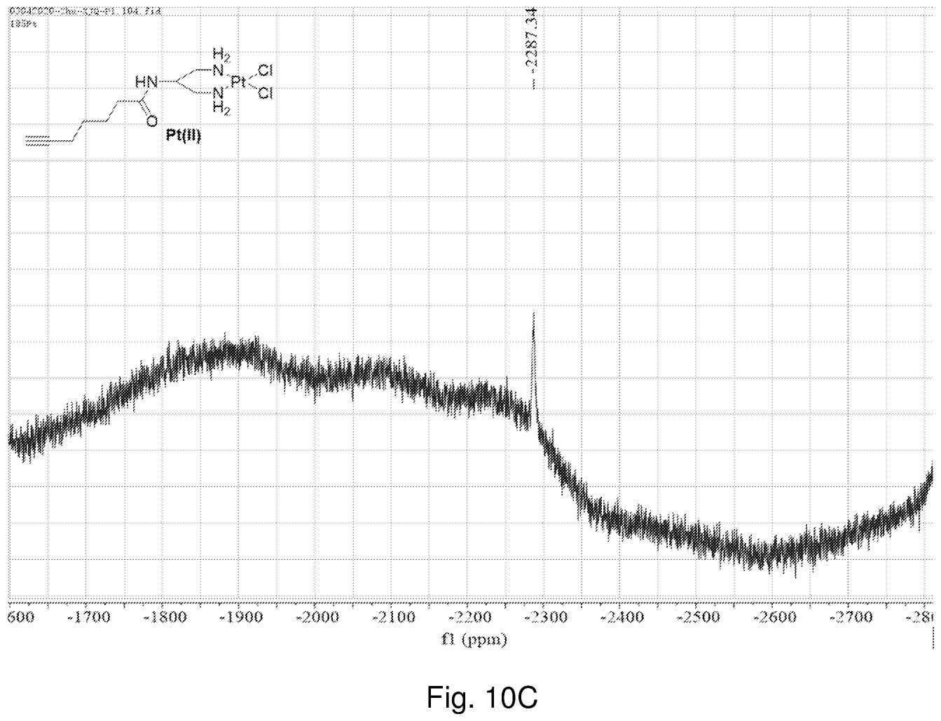

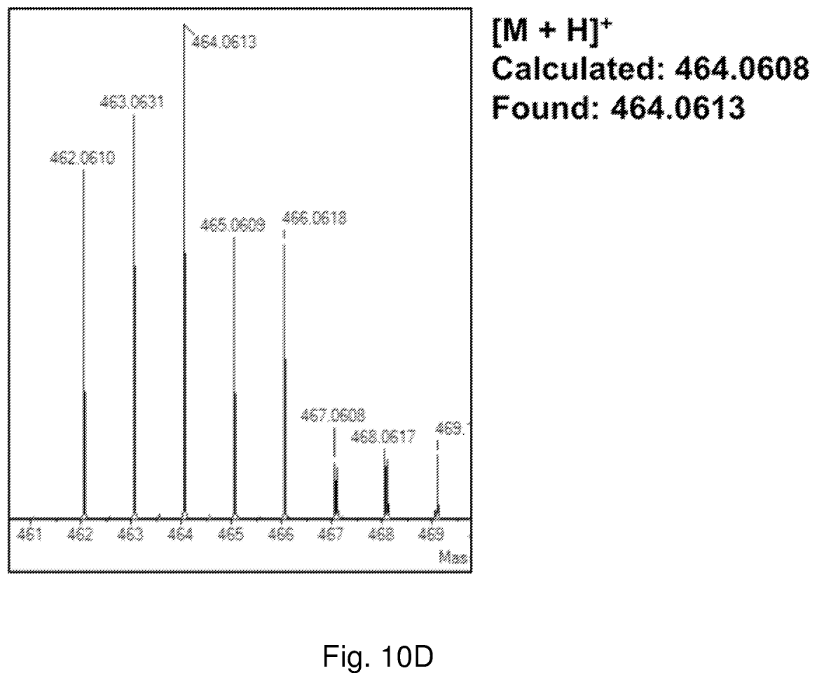

To a solution of NP (22.3 mg, 0.1 mmol) in anhydrous DMF was added DBU (29.9 μL, 0.2 mmol), then compound 11 (42.2 mg, 0.1 mmol). The mixture solution was stirred in the dark at room temperature for 48 h. Then water was then added to the reaction mixture and the clear-brown solution was refrigerated overnight. The solid was isolated by filtration, washed with water, ethanol, Et₂O and dried in vacuum to yield alkyne-Pt(II) as yellow powder. ¹H NMR (600 MHZ, DMF-d7) δ 7.96 (d, J=7.8 Hz, 1H), 5.25 (d, J=42.0 Hz, 4H), 4.19-4.11 (m, 1H), 2.82 (dt, J=9.3, 3.5 Hz, 2H), 2.75 (d, J=2.7 Hz, 1H), 2.74-2.69 (m, 2H), 2.25-2.19 (m, 4H), 1.68 (p, J=7.5 Hz, 2H), 1.51 (p, J=7.2 Hz, 2H) (FIG. 10A). ¹³C NMR (151 MHZ, DMF-d7) δ 173.32, 85.69, 71.72, 48.77, 48.12, 26.08, 19.21 (FIG. 10B). ¹⁹⁵Pt NMR (129 MHZ, DMF-d7): δ −2287.34 (s) (FIG. 10C). ESI-HRMS (positive mode) m/z: [M+H]⁺ calcd. for C₁₀H₁₉Cl₂N₃OPt: 464.0608, found: 464.0613 (FIG. 10D).

### Example 3

### Structural Characterization

Three of the representative Pt(II) anticancer drugs, including cisplatin, carboplatin, and oxaliplatin that are used in clinics worldwide, as well as transplatin, were selected as the equatorial cores of the Pt(IV) structures of the present invention. Molecular scaffolds lacking strong absorbance at 365 nm, in particular, acetic acid, succinic acid, and succinic ester, were selected as innocent axial ligands.

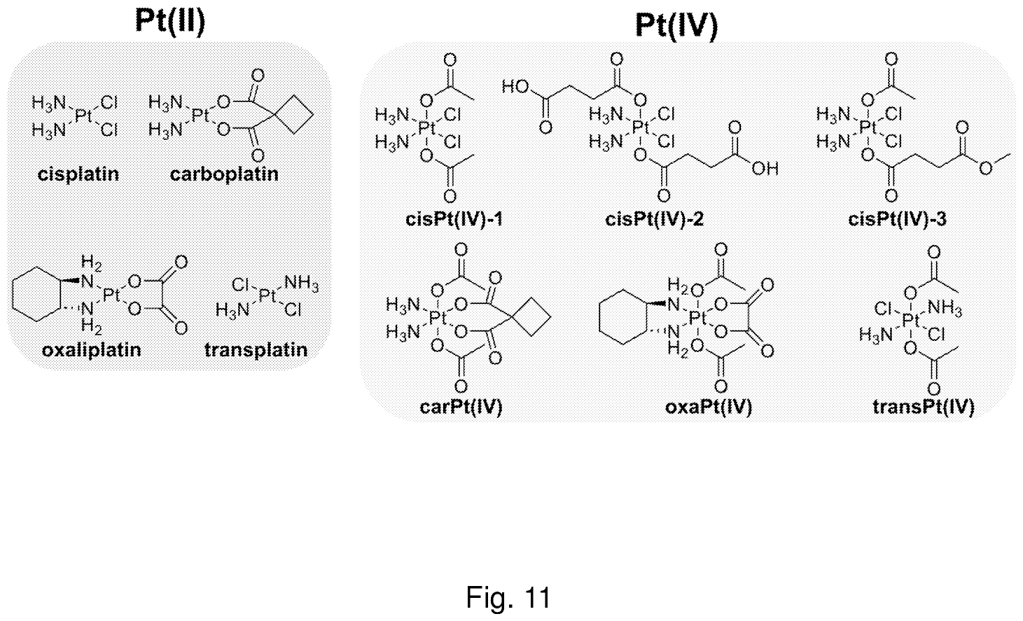

Six Pt(IV) complexes were readily obtained in this example by functionalizing the hydroxyl group of dihydroxido Pt(IV) species with anhydrides or NHS esters via the carboxylate linkage (FIG. 11). The synthesized complexes were fully characterized by electrospray ionization high-resolution mass spectrometry (ESI-HRMS) and multinuclear NMR spectroscopy (¹H, ¹³C, and ¹⁹⁵Pt), and the purities of the complexes were determined by analytical reversed-phase HPLC (RP-HPLC, 254 nm; >95%) (FIGS. 1A-1E, 2A-2E, 3A-3E, 4A-4E, 5A-5E, and 6A-6E).

### Example 4

### Photolysis Properties

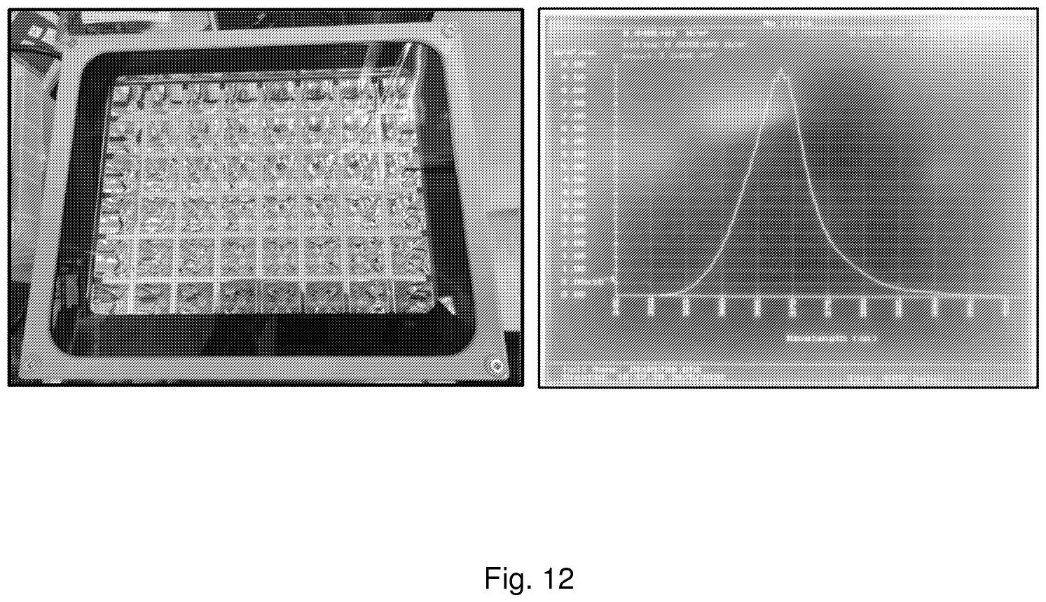

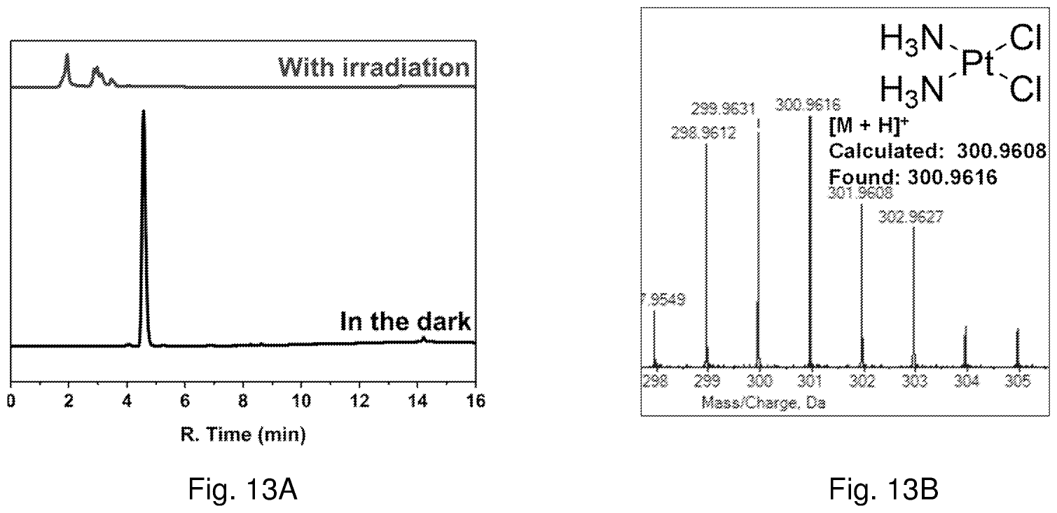

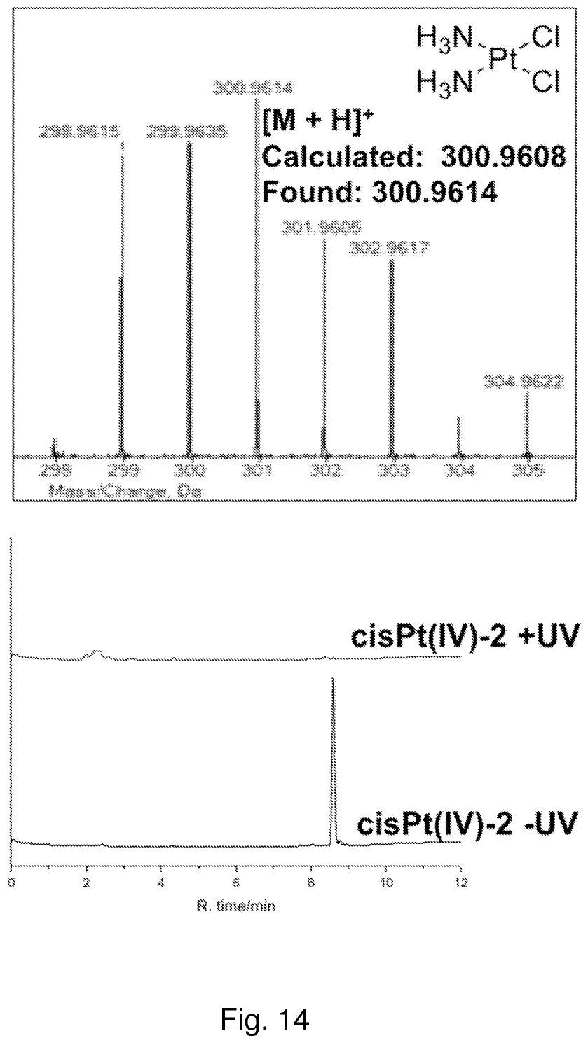

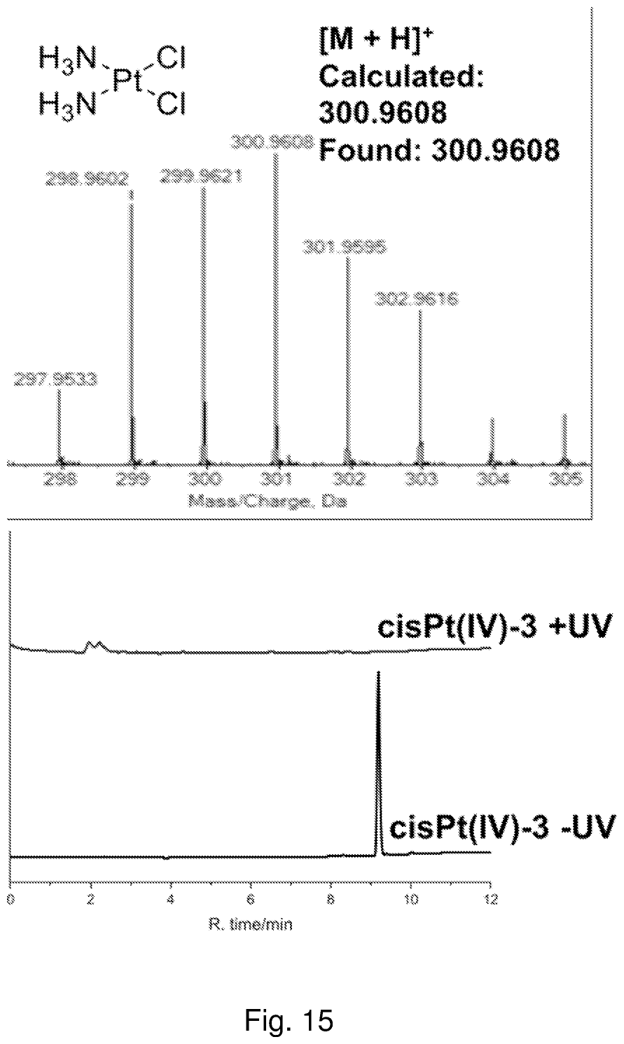

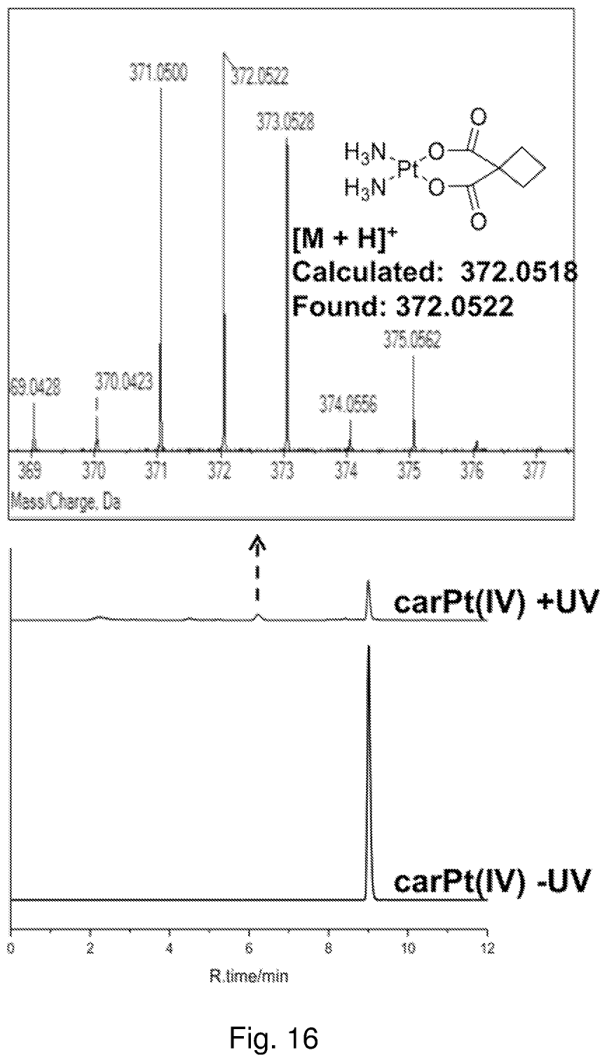

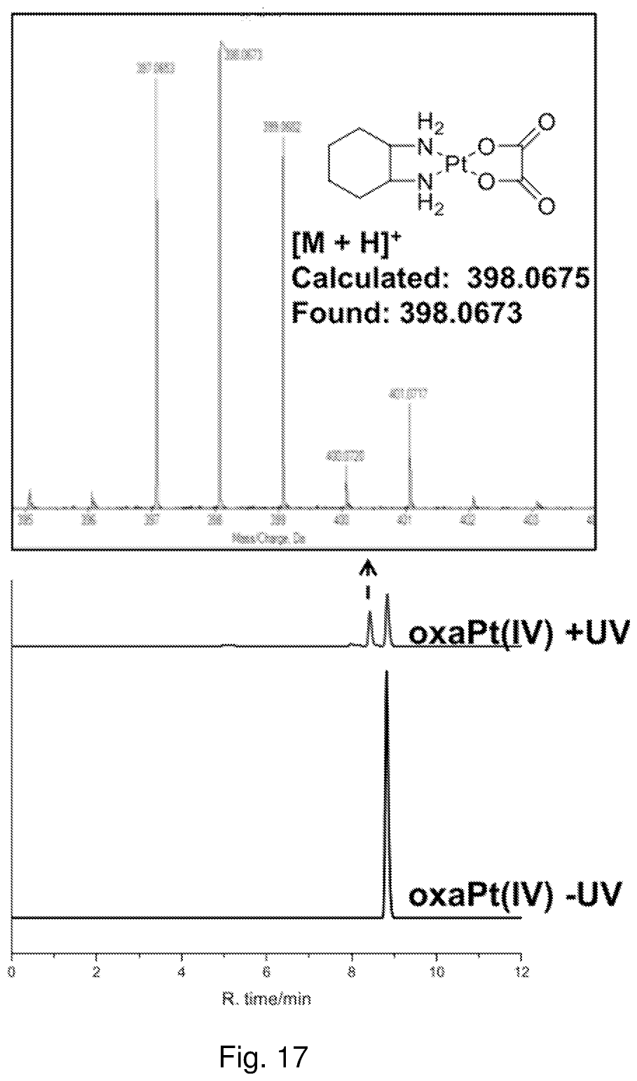

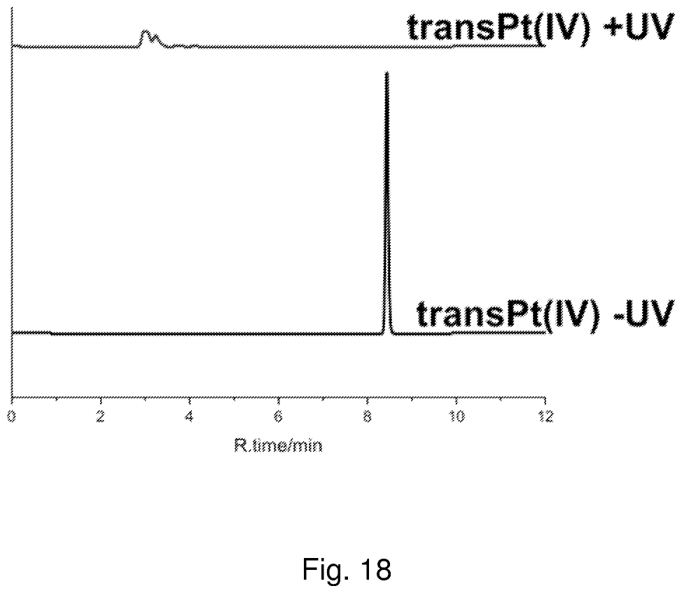

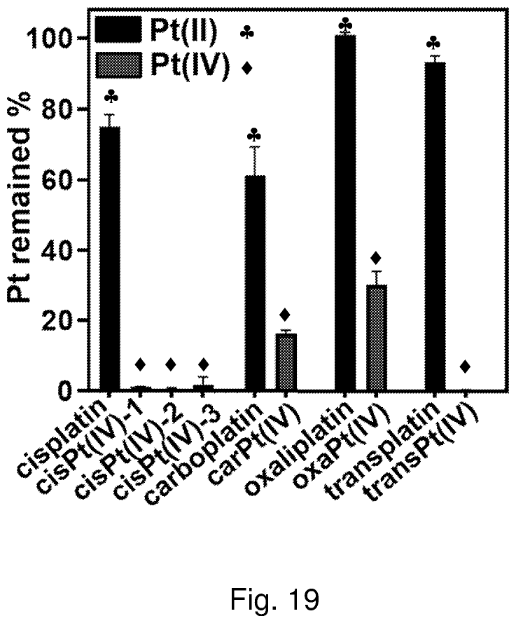

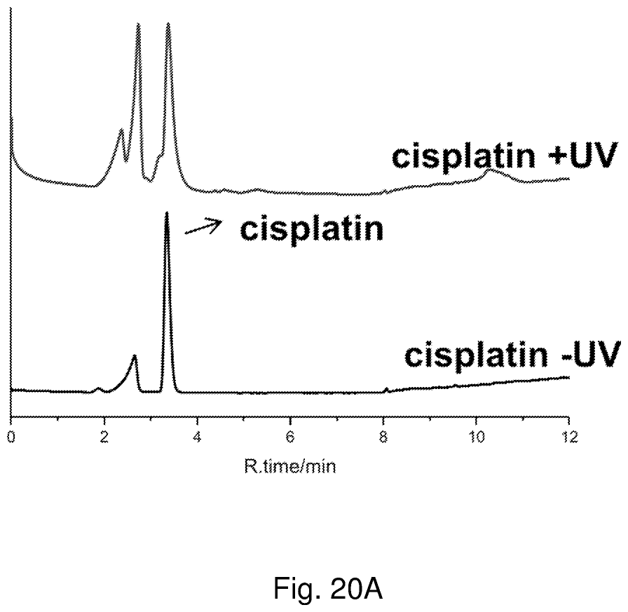

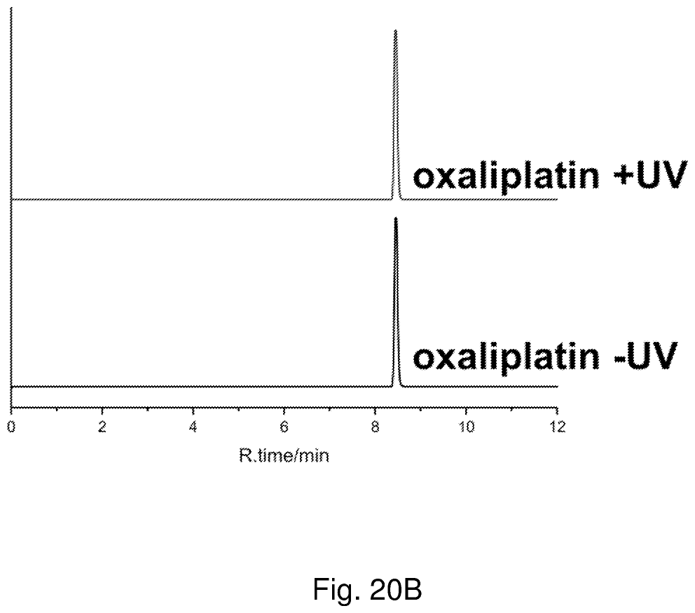

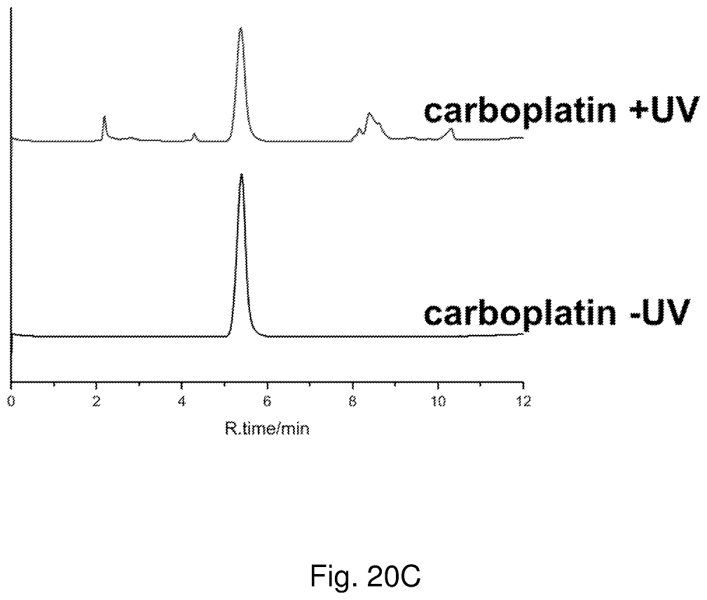

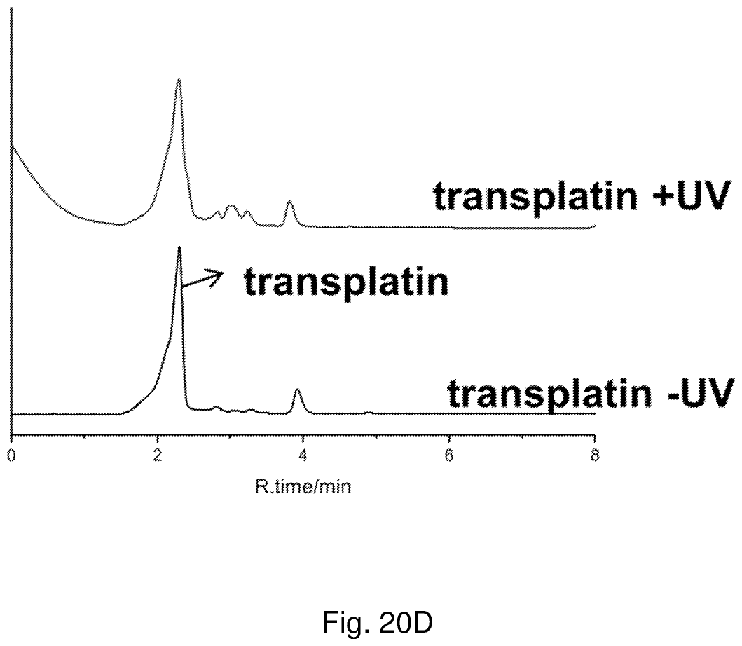

The photolysis properties of the Pt(IV) complexes in water were investigated using HPLC. Upon irradiation by 365 nm UV light (FIG. 12) at a power density of about 9.8 mW/cm² for 10 min, the peak height of all Pt(IV) complexes in the HPLC chromatogram decreased significantly (FIGS. 13A-13B, 14-18), and various new species emerged, indicating the rapid photolysis of the Pt(IV) complexes. In contrast, the corresponding Pt(II) complexes were relatively resistant to photolysis; for instance, 75% of cisplatin and 99% of oxaliplatin remained stable under the experimental conditions (FIGS. 19 and 20A-20D). A similar photolysis properties was observed for transplatin and transPt(IV) (FIGS. 14-19, and 20A-20D). The formation of Pt(II) counterpart from the Pt(IV) complexes with a cis geometry was confirmed using HPLC and ESI-HRMS (FIGS. 13B and 14-18).

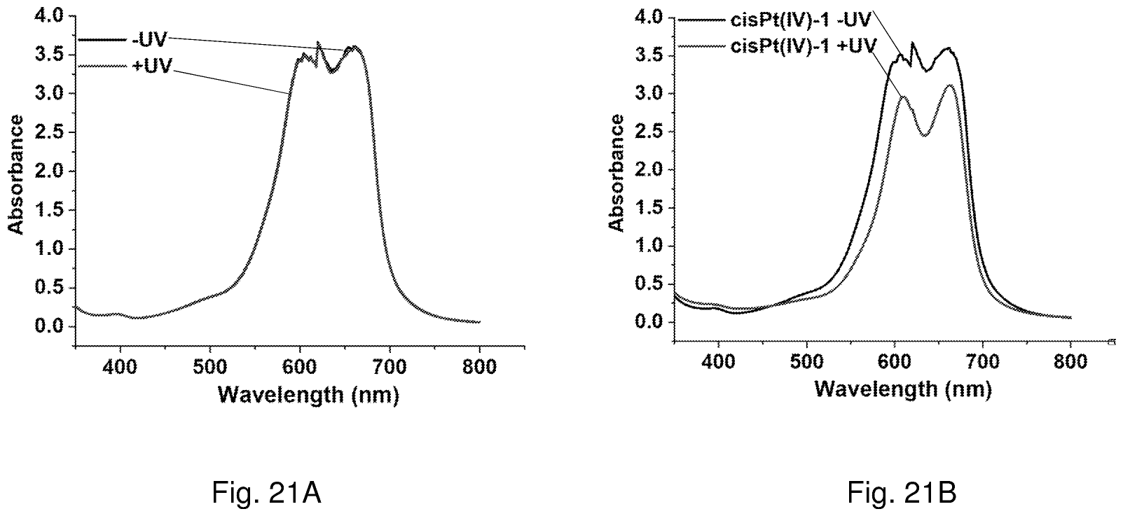

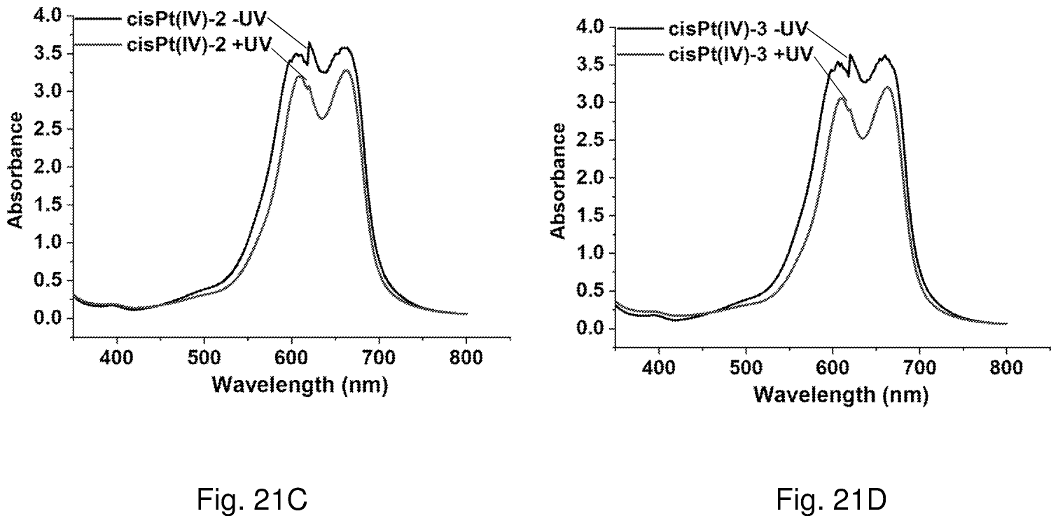

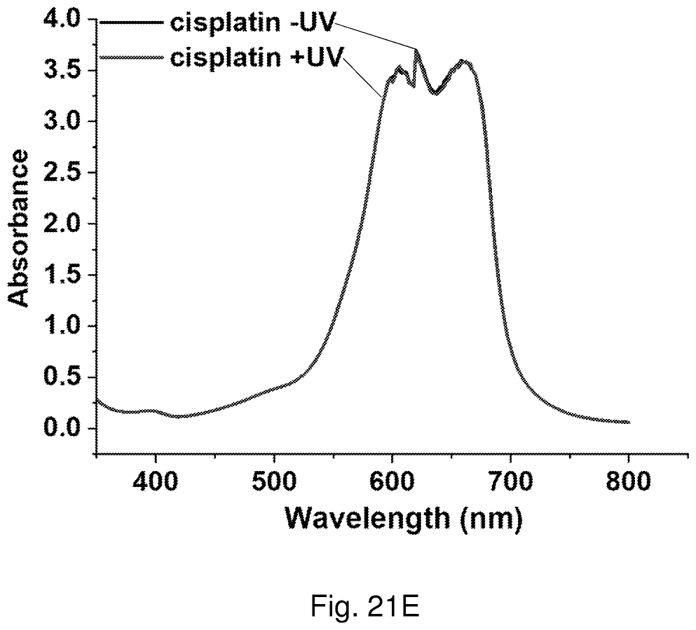

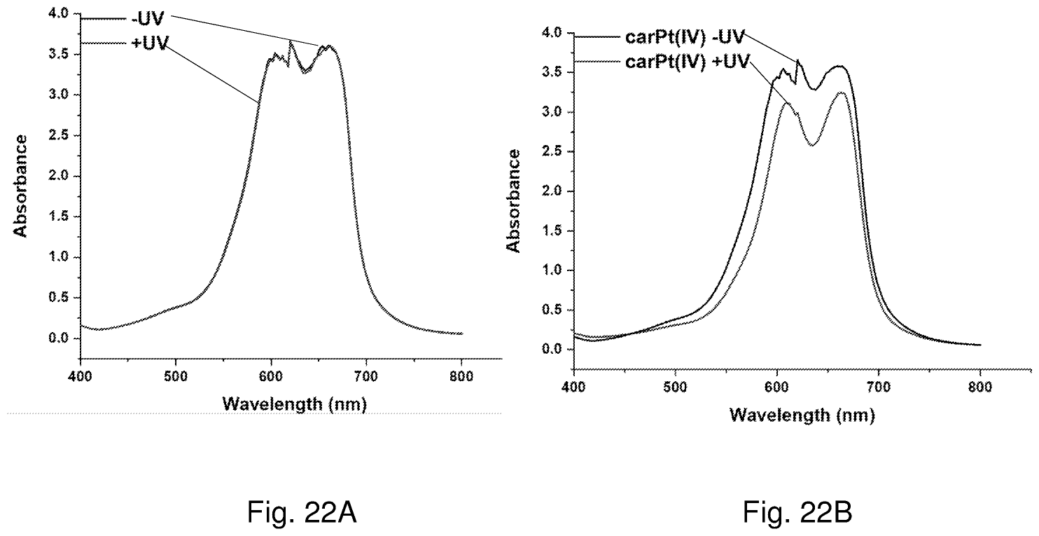

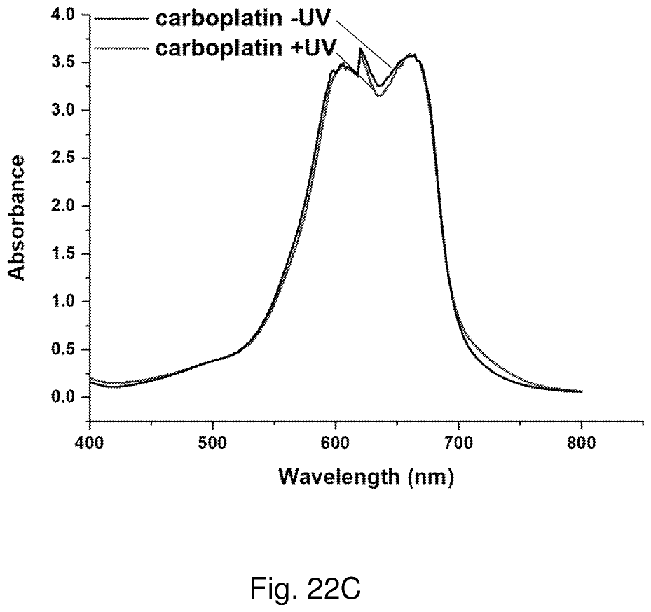

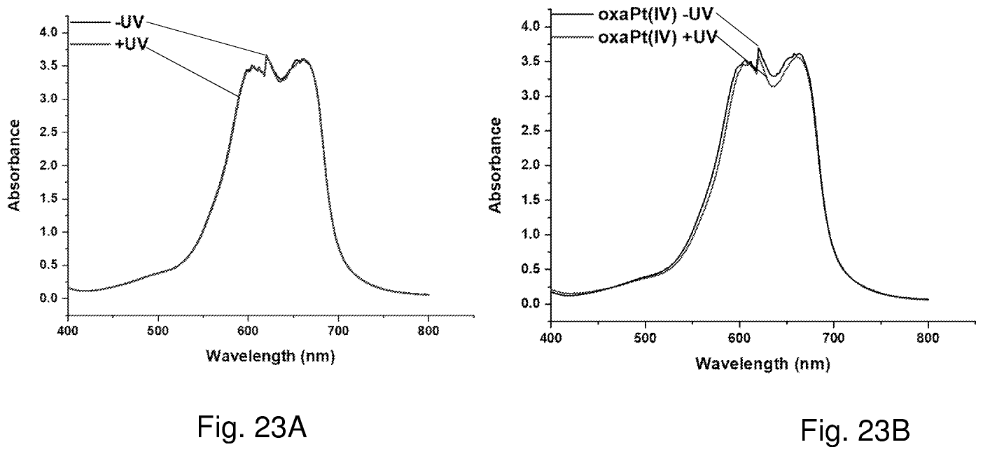

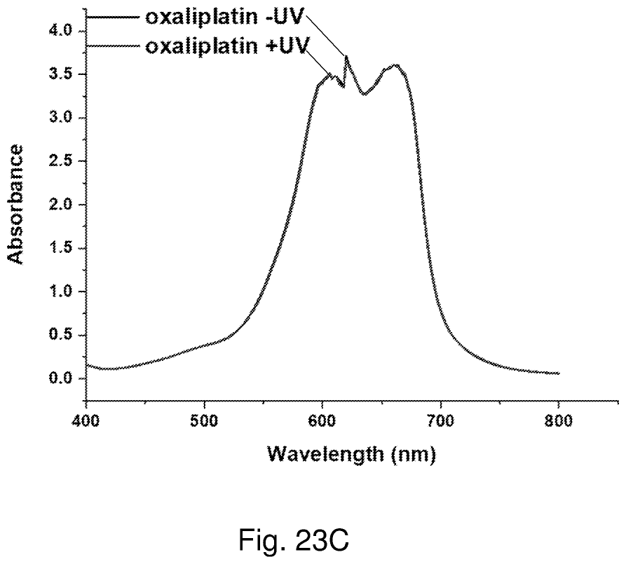

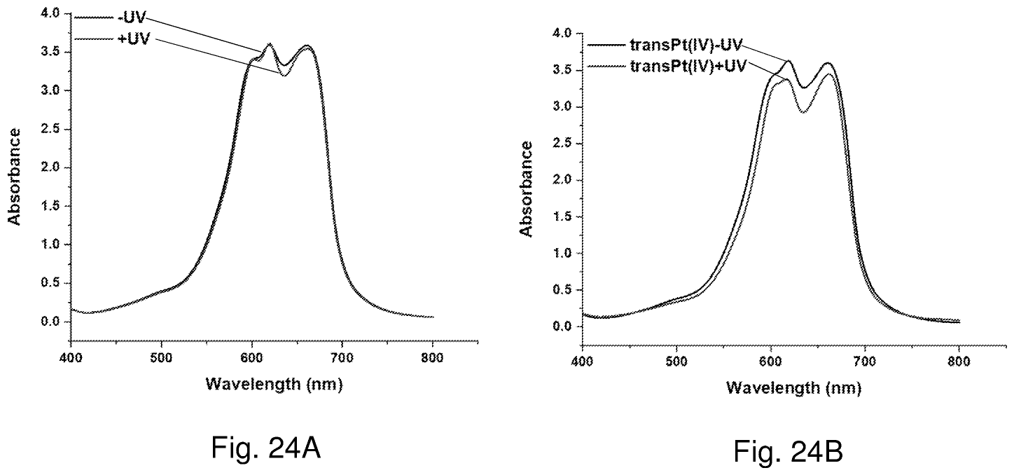

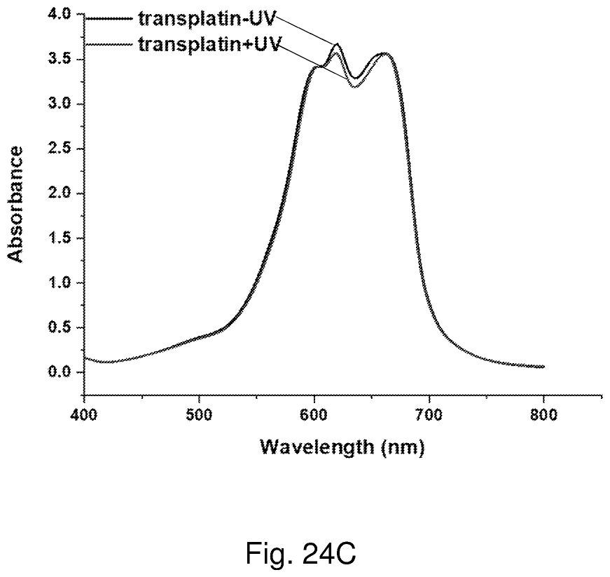

It is believed that the photolysis of the Pt(IV) complexes may involve a radical-forming mechanism. Accordingly, the photolysis mechanism of the Pt(IV) complexes was investigated. The generation of radicals following photolysis of the complexes was first determined by methylene blue (MB) degradation experiments. As shown in FIGS. 21A-21E, 22A-22C, 23A-23C, and 24A-24C, MB was fully degraded in the Pt(IV) complex-treated group following 10 mins of irradiation. In contrast, MB remained intact in Pt(II) complex-treated samples. These results suggest the production of reactive radical species following the photolysis of Pt(IV) complexes but not the Pt(II) counterparts. A similar effect was also observed for transplatin and transPt(IV) (FIGS. 24B and 24C), implying that the oxidation state rather than the coordination geometry determines the ability to generate reactive radical species.

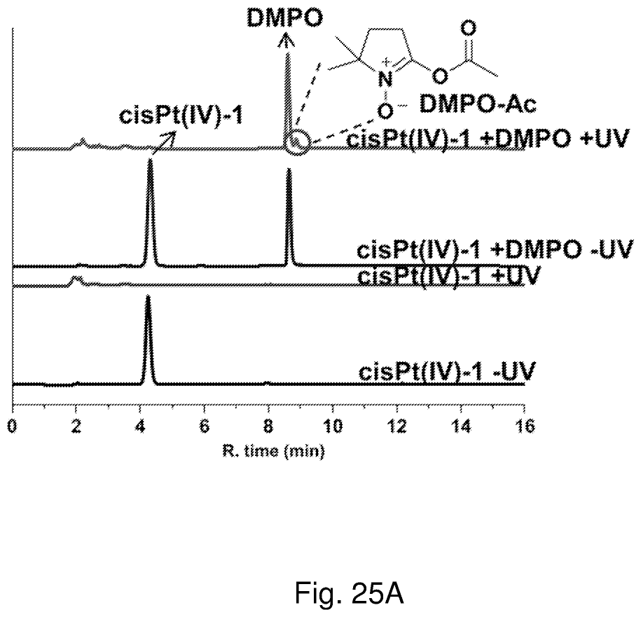

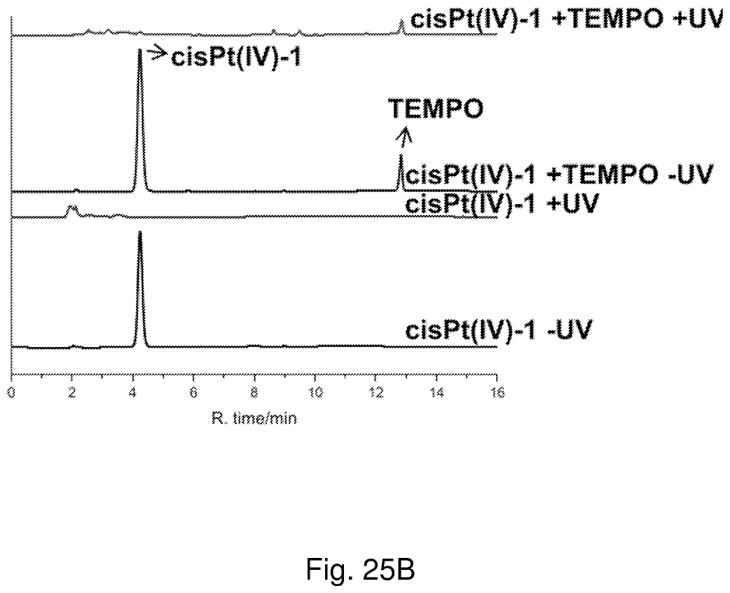

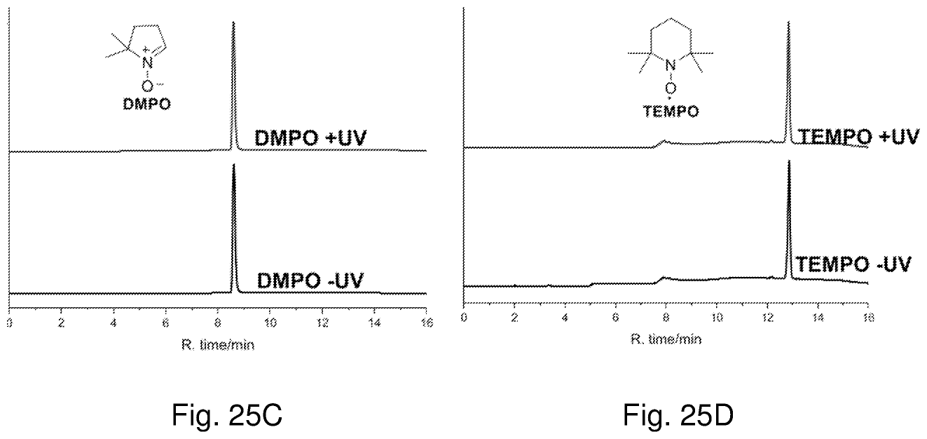

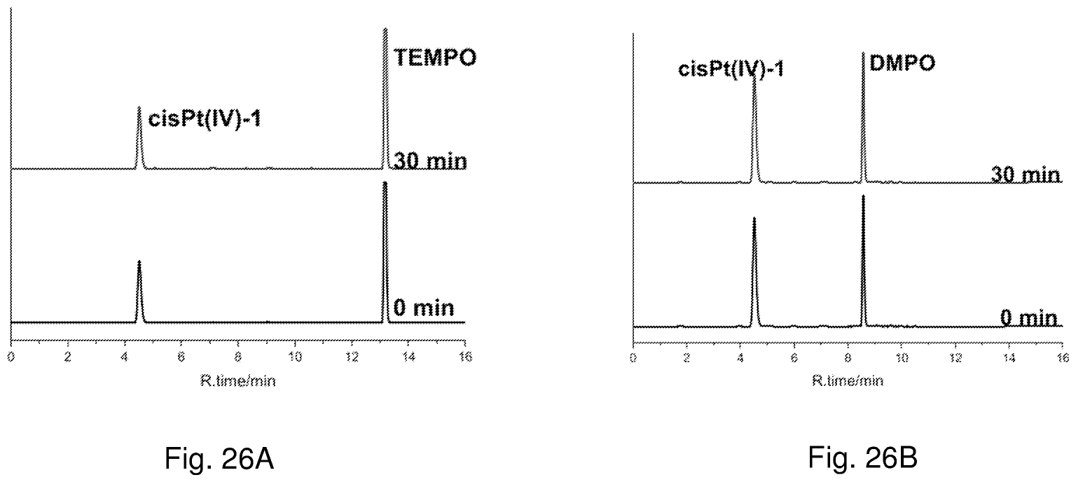

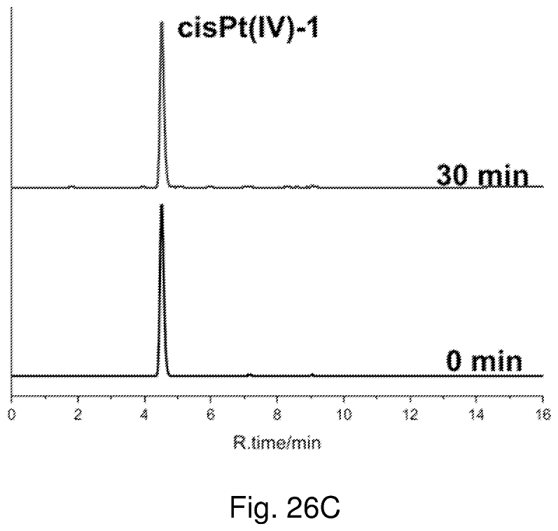

To further explore the photolysis mechanism, radical spin trap experiments were conducted. Cis-Pt(IV)-1 was used as an example complex for the experiment. Typically, the photolysis of the cis-Pt(IV)-1 complex was monitored in the presence of the two common spin traps: 5,5-dimethyl-1-pyrroline N-oxide (DMPO) and (2,2,6,6-tetramethylpiperidine-1-yl)oxyl (TEMPO). As shown in FIGS. 25A and 25B, different new species were observed in the presence of the radical spin traps after 10 mins of irradiation. In contrast, the radical spin traps themselves were stable upon irradiation, and the free cisPt(IV)-1 as well as its mixture with radical spin traps remained intact in the dark for about 30 min (FIGS. 25C-25D and 26A-26C), showing that the radical species were formed as a result of the photolysis of cisPt(IV)-1.

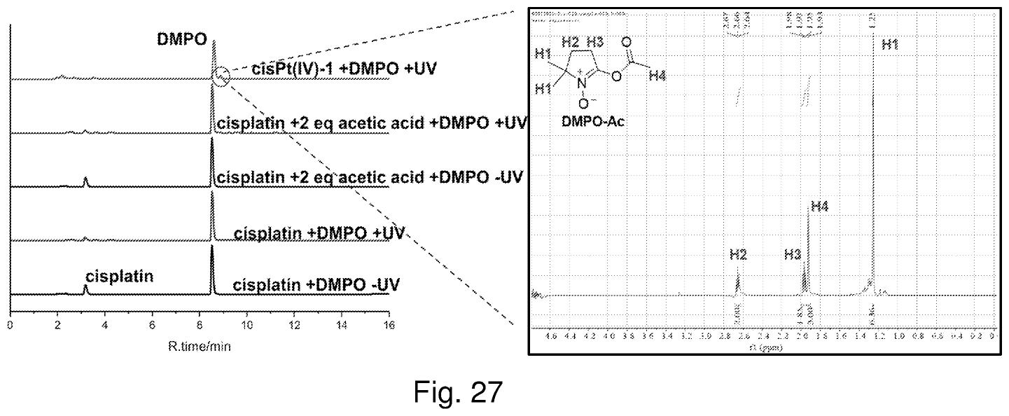

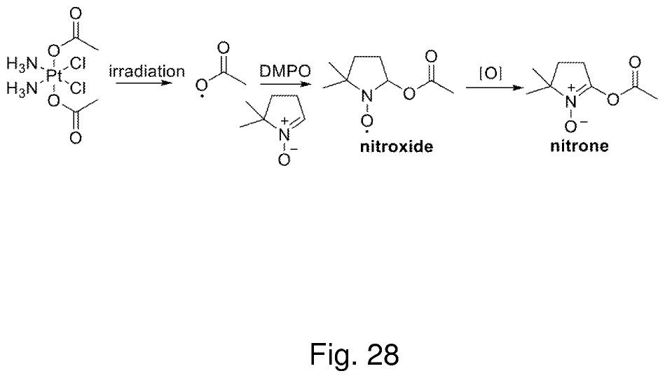

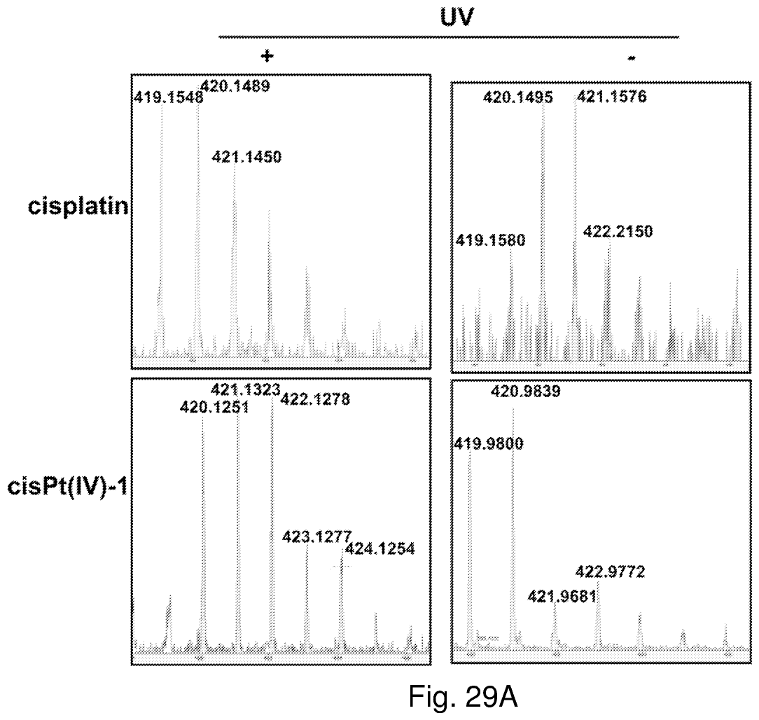

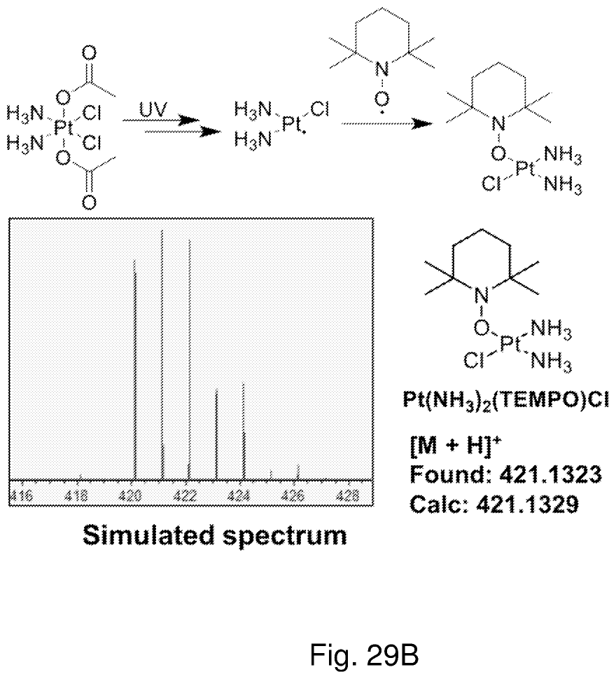

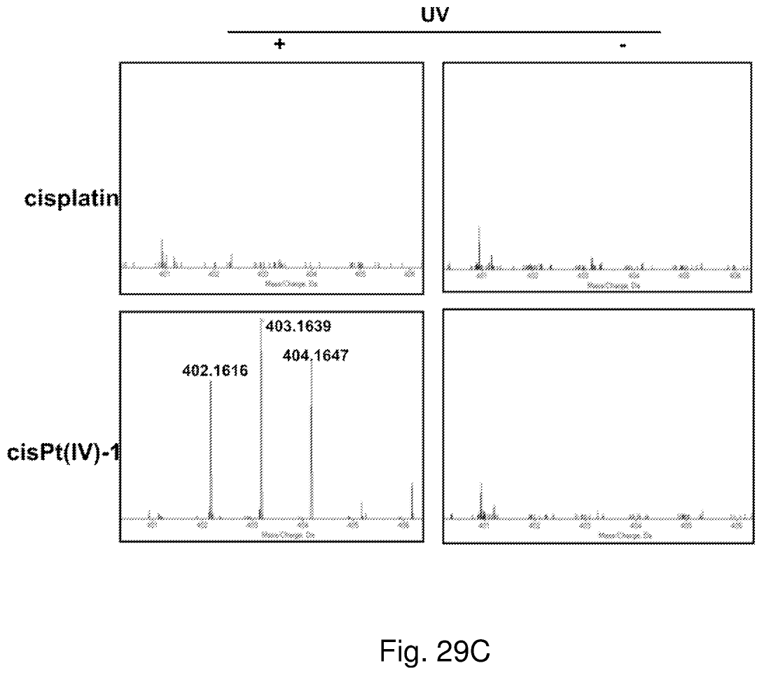

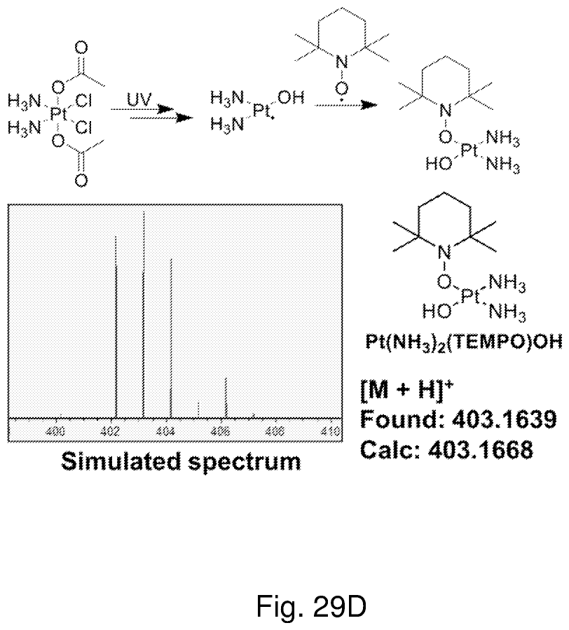

Notably, a new peak at 8.9 min appeared in the HPLC chromatogram of the mixture of DMPO and cisPt(IV)-1 upon irradiation, which was subsequently separated and characterized as the spin adduct of DMPO with acetato ligand (DMPO-Ac), evidencing the formation of acetate radicals (FIGS. 25A and 27-28). The platinum-containing spin adducts of TEMPO were further identified by platinum's characteristic isotopic pattern via ESI-HRMS. Two spin adducts of TEMPO with platinum, namely [Pt(NH₃)₂(TEMPO)Cl] and [Pt(NH₃)₂(TEMPO)OH], were observed in the solution of cisPt(IV)-1 upon irradiation, indicating the formation of Pt(I) radicals (FIGS. 29A-29D). In contrast, these spin-adduct products with platinum could not be observed in the Pt(II)-treated groups (FIGS. 27-28 and 29A-29D), indicating that these radical species originated directly from the photolysis of Pt(IV) complexes rather than the resultant Pt(II) counterpart.

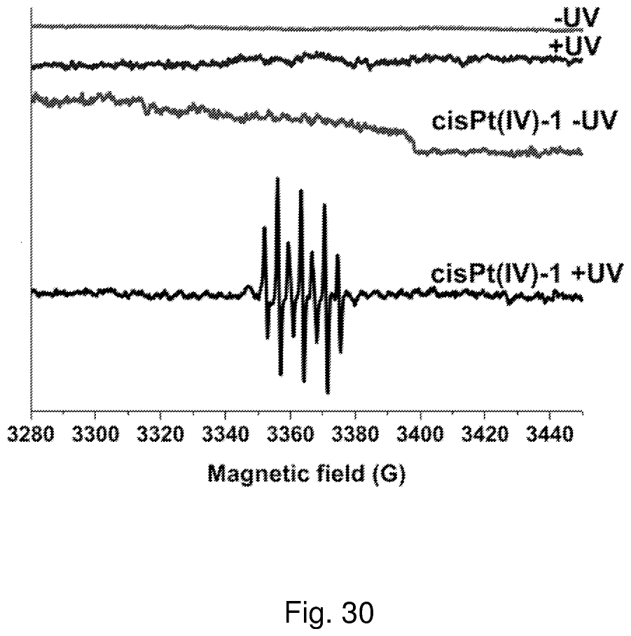

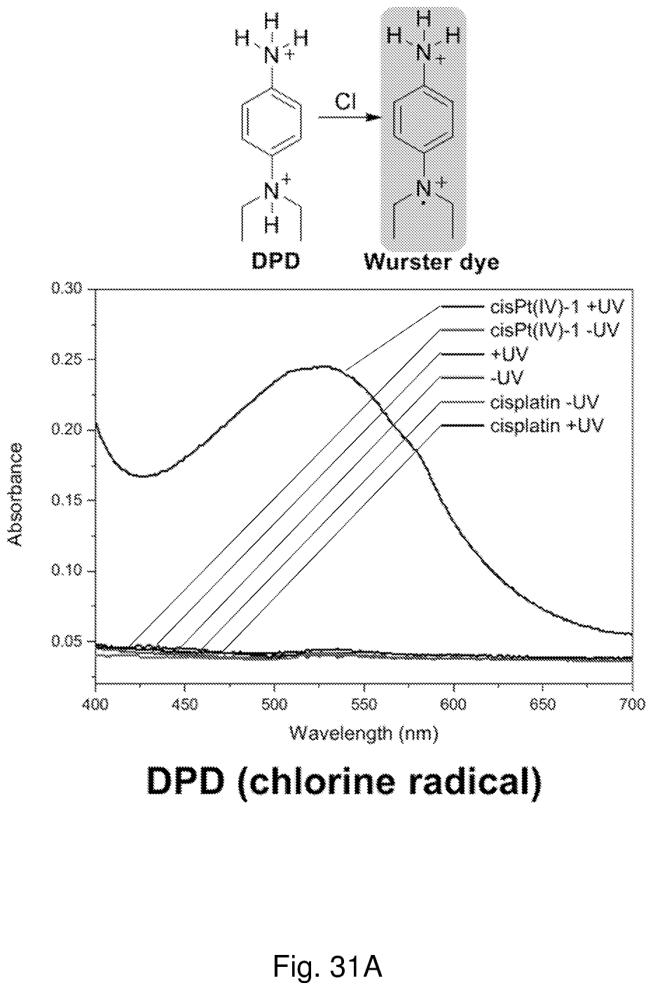

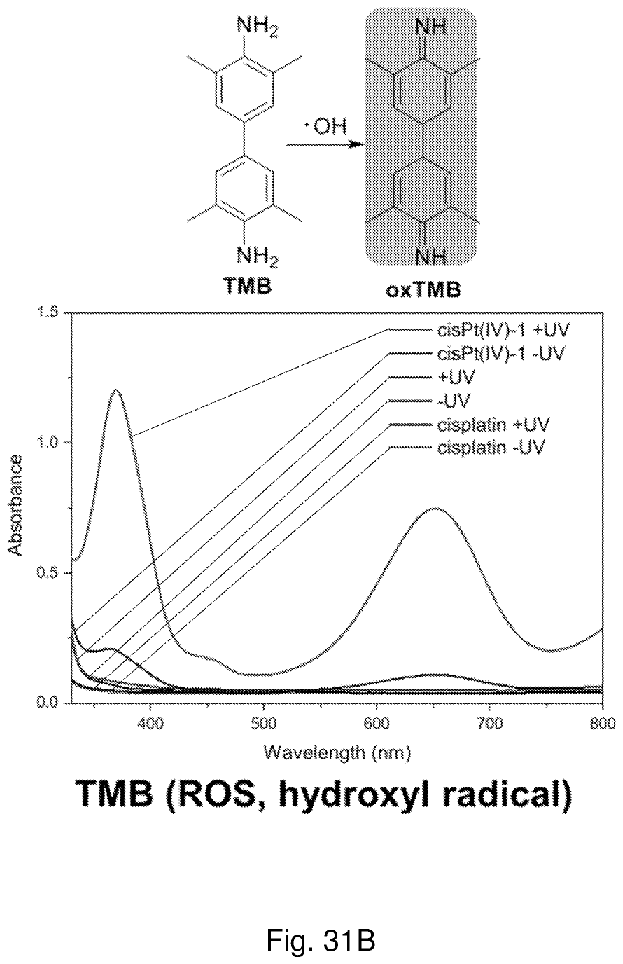

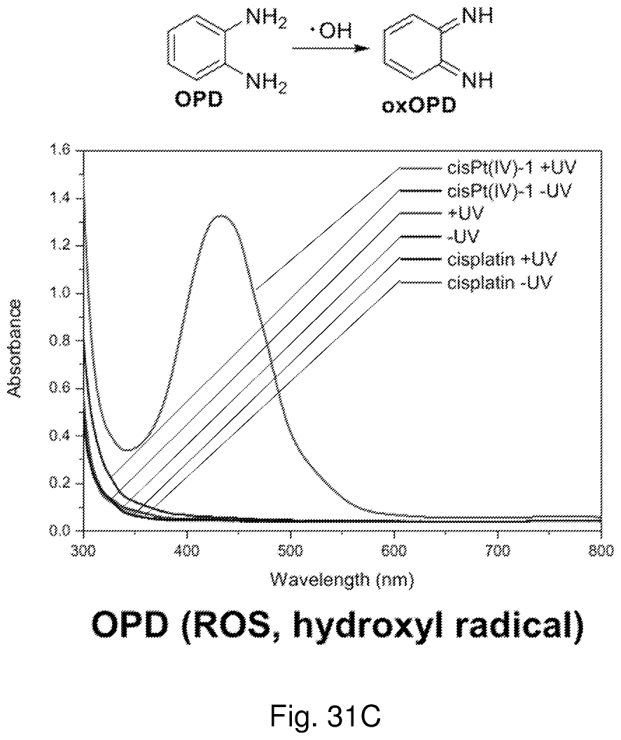

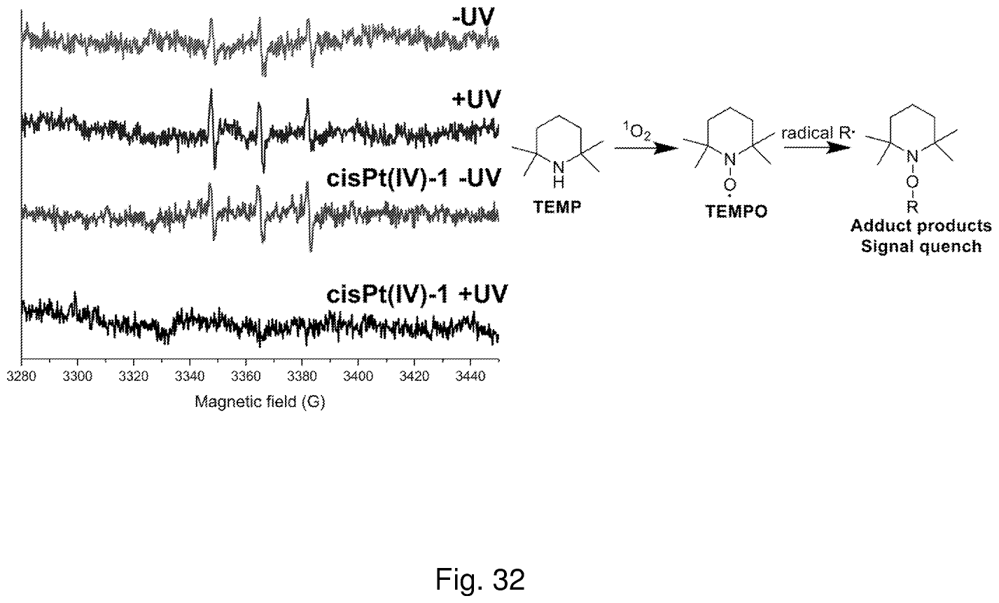

To reveal more about the reactive radical species, electron paramagnetic resonance (EPR) measurements using DMPO and (2,2,6,6-tetramethylpiperidine)oxyl (TEMP) as the radical spin traps. As shown in FIG. 30, a seven-line EPR spectrum of DMPOX, which was identified as the oxidized derivative of DMPO by chlorine radicals, appeared in the cisPt(IV)-1-treated group upon irradiation, suggesting the presence of chlorine radicals during the photolysis process. The N,N-diethyl-p-phenylenediamine (DPD) standard method was then used to verify the formation of chlorine radicals. An absorbance spectrum peak at 515 nm, known to correspond to Würster dye, a magenta colored compound formed by the reaction between colorless DPD and free chlorine, was observed in the cisPt(IV)-1-treated group upon irradiation, but not the cisplatin-treated group (FIGS. 31A-31C), confirming the production of chlorine radicals following photolysis. In addition, a three-line signal, corresponding to TEMP adducts with singlet oxygen (TEMPO), disappeared in the solution of cisPt(IV)-1 upon irradiation (FIG. 32). Given that radical species quench TEMPO, it further suggests the production of radical species during the photolysis of the Pt(IV) complexes.

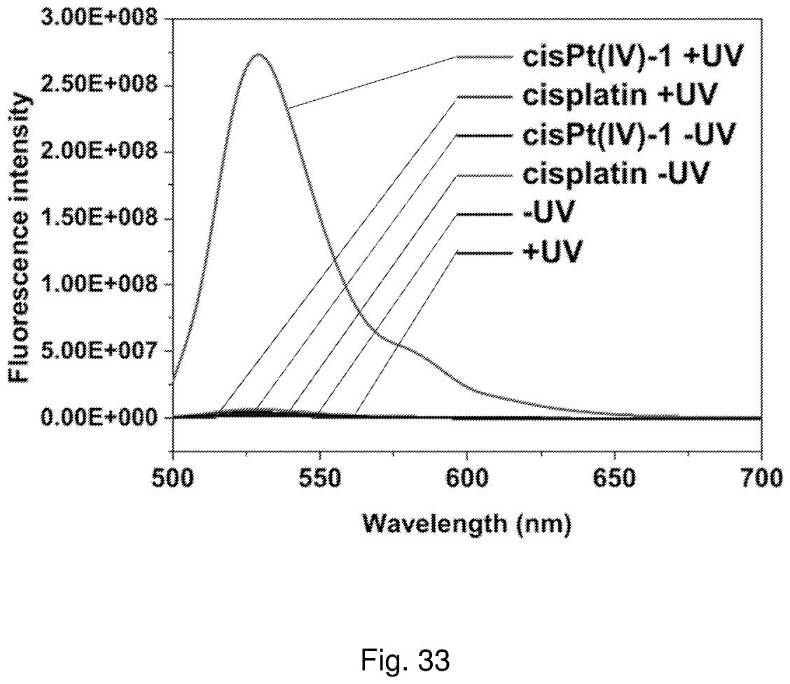

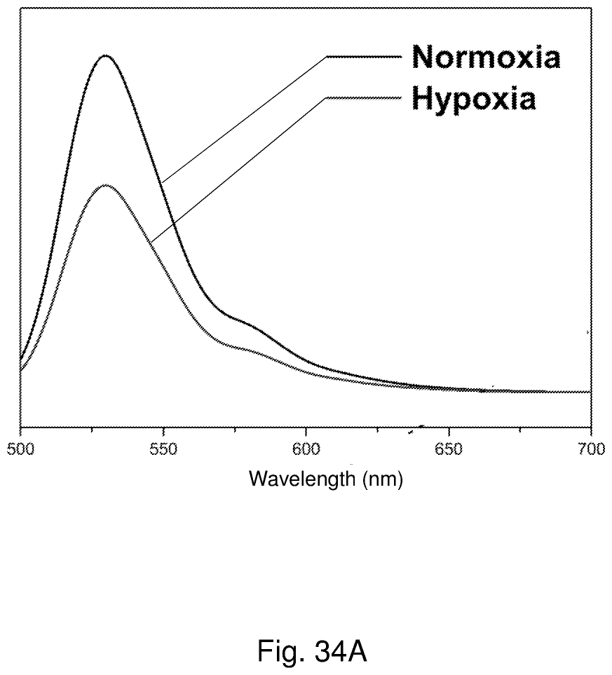

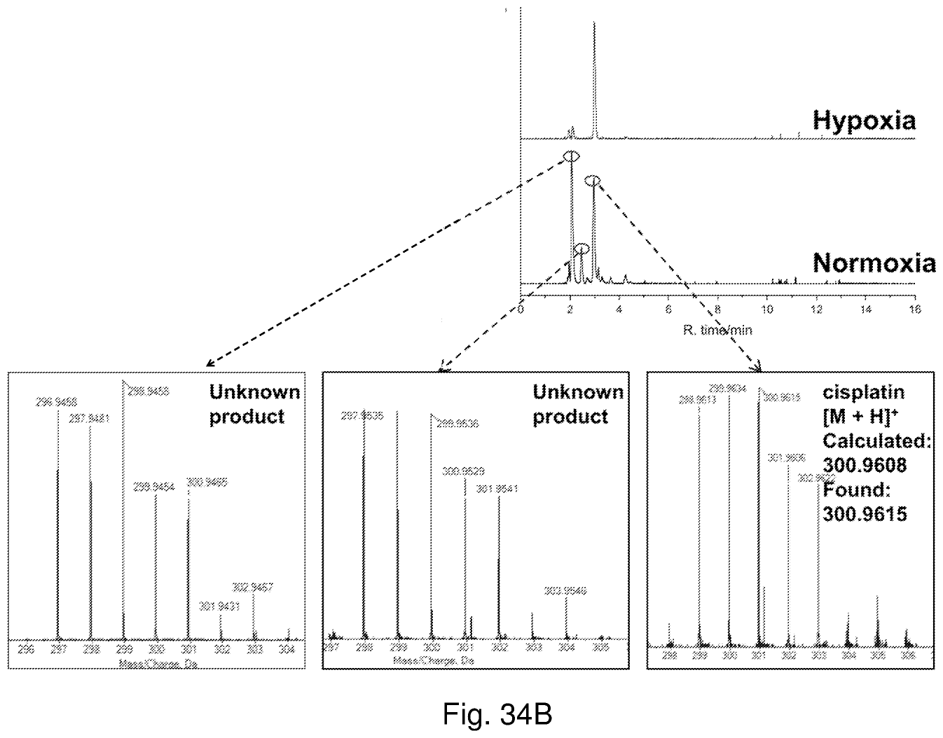

Given the fact that cisPt(IV)-1 generates reactive radical species during photolysis, it is believed that reactive oxygen species (ROS) may be further generated in the presence of oxygen and water. As a support. 2′7′-dichlorofluorescein diacetate (DCFH-DA) was used as an ROS probe to detect any ROS generated upon photolysis of cisPt(IV)-1. As shown in FIG. 33, the solution of cisPt(IV)-1 exhibited significantly increased fluorescence intensity upon irradiation (compared with the samples treated with cisplatin), suggesting the formation of ROS following the photolysis of cisPt(IV)-1. These results were further confirmed by using two colorimetric hydroxyl radical probes, 3,5,3′,5′-tetramethylbenzidine (TMB) and o-phenylenediamine (OPD). As shown in FIGS. 31A-31C, characteristic absorption peaks at 652 nm and 440 nm, of which are the oxidation products of TMB (oxTMB) and OPD (oxOPD) by hydroxyl radicals, respectively, were observed in the cisPt(IV)-1-but not cisplatin-treated group upon irradiation. It should be noted that under hypoxia, less ROS was generated than under normoxic conditions, as reflected in a decreased fluorescence intensity of DCFH-DA (FIG. 34A) and different patterns of photolysis products (FIG. 34B), suggesting that oxygen is indeed involved in the photolysis process of cisPt(IV)-1.

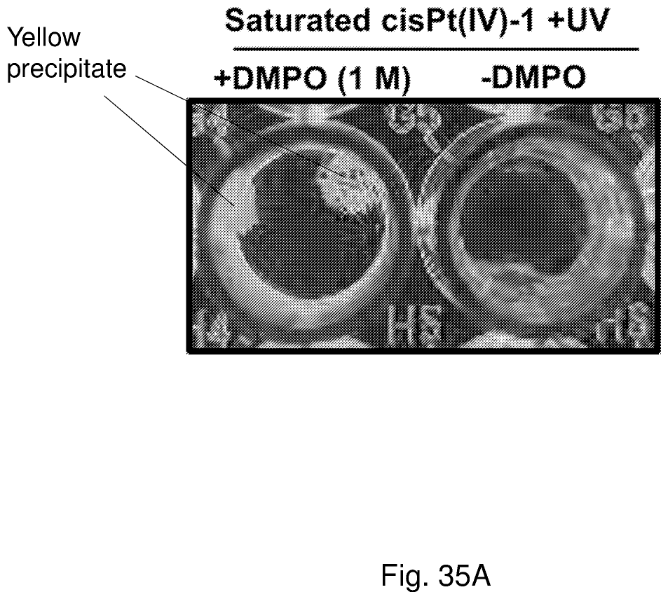

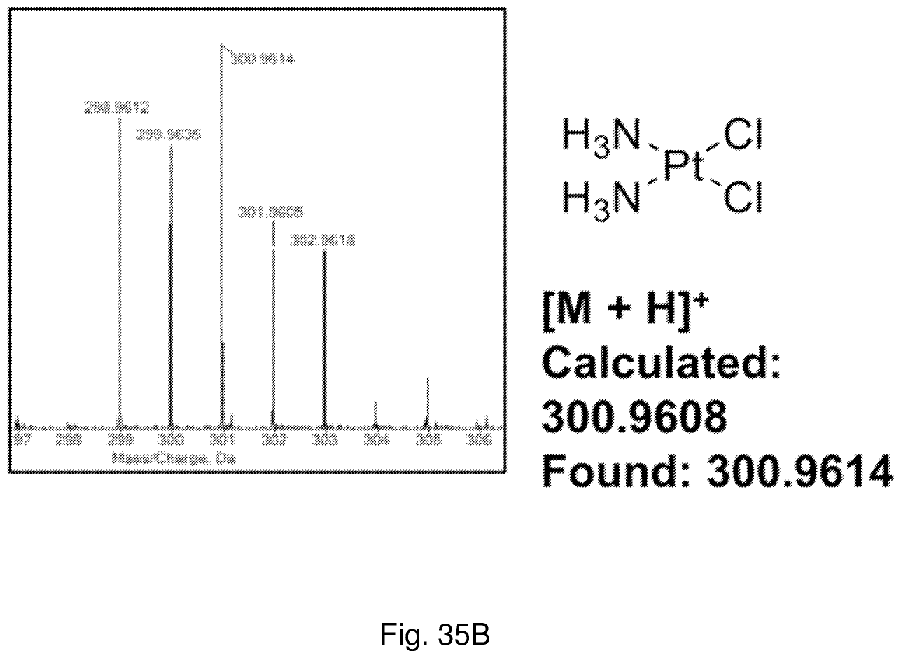

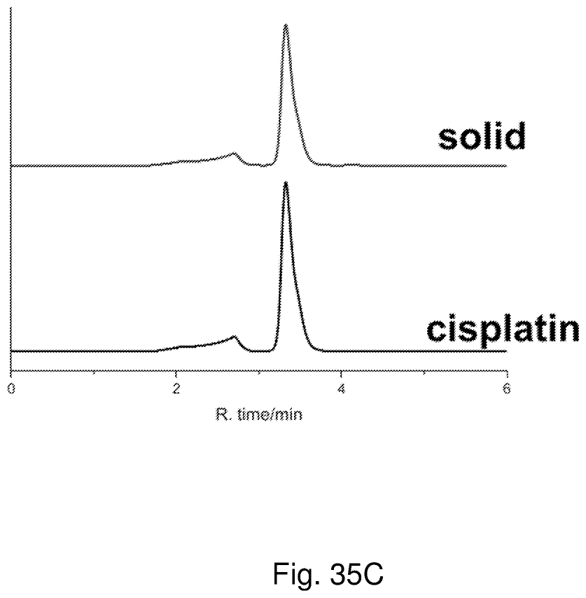

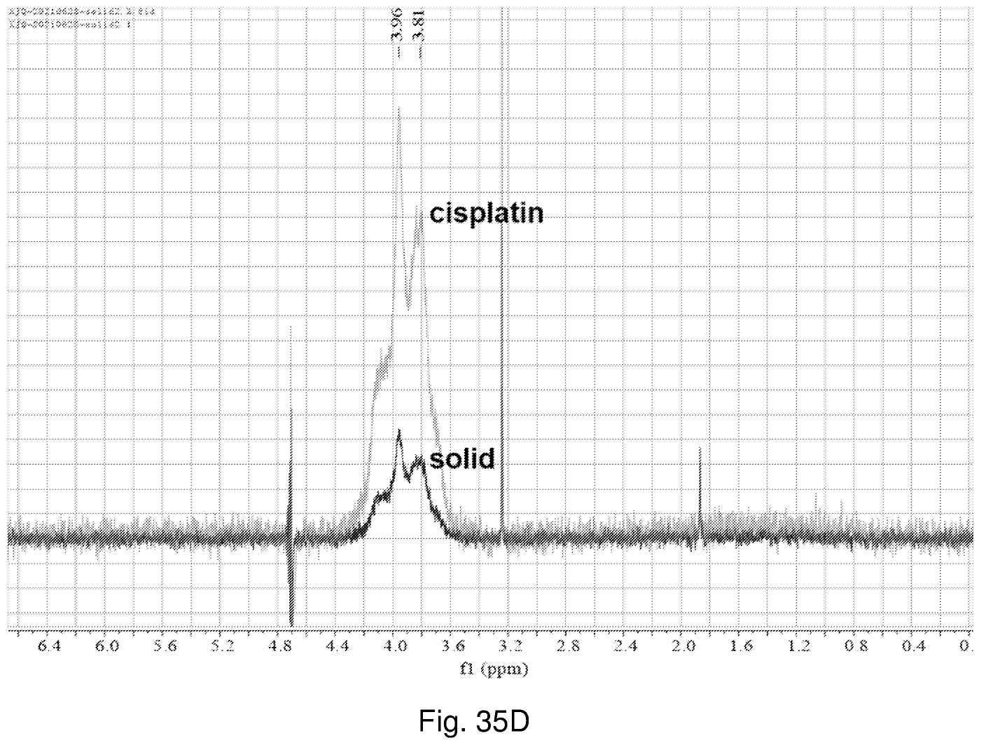

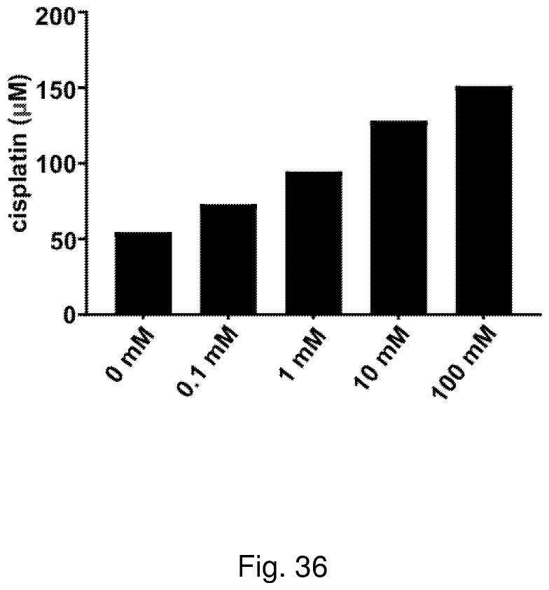

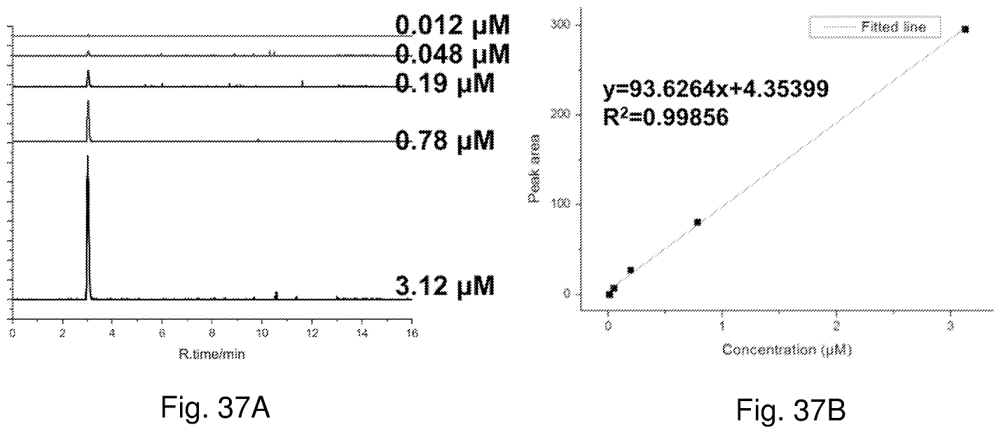

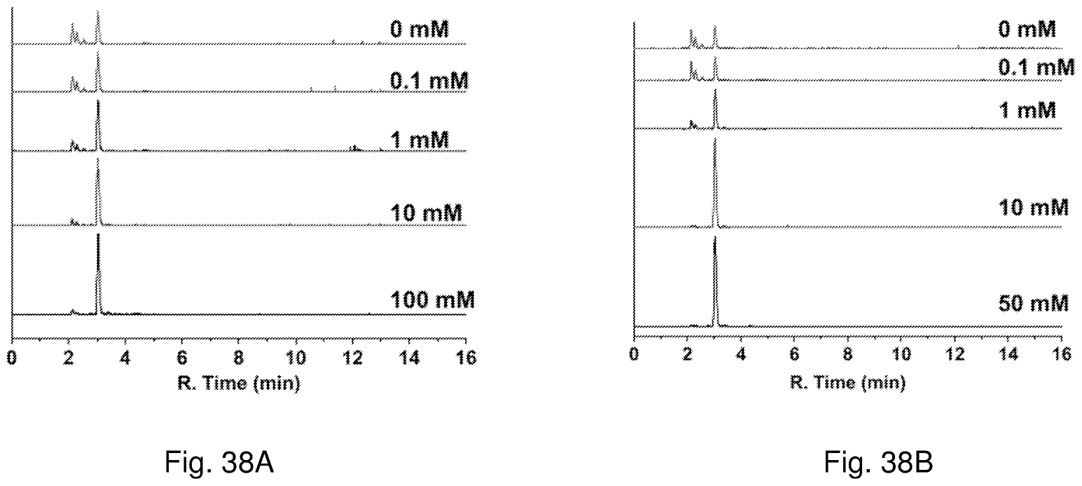

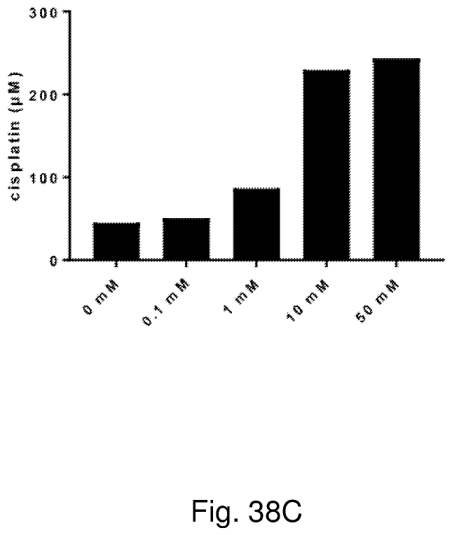

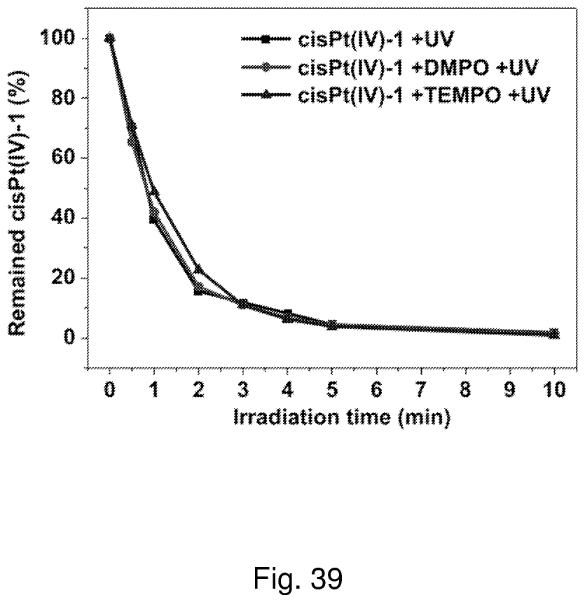

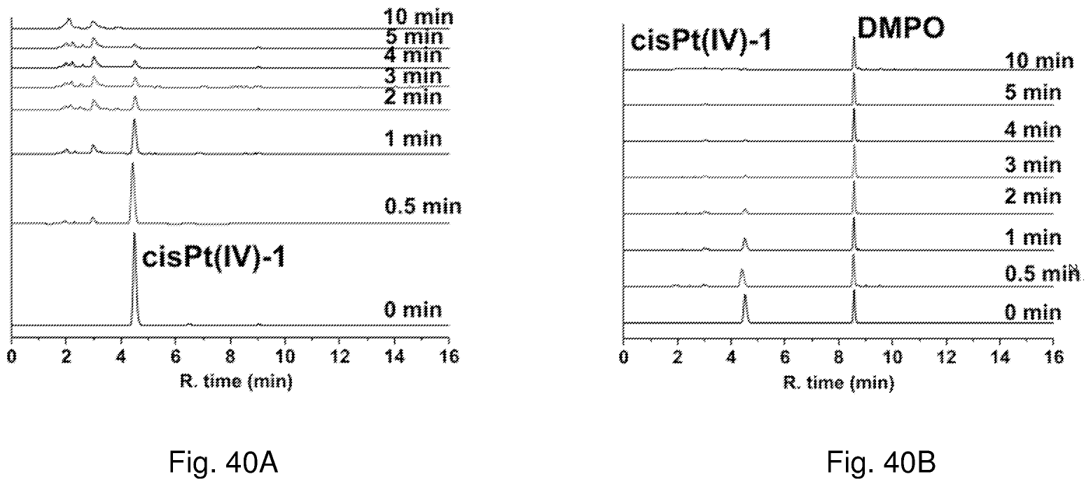

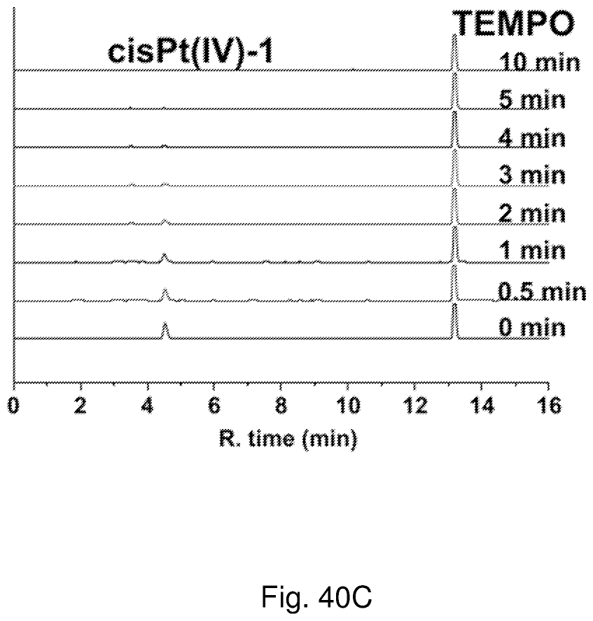

Unexpectedly, it is found that in a saturated cisPt(IV)-1 solution in water containing DMPO, a yellow precipitate was visually observed following irradiation (FIG. 35A), which was determined to be cisplatin using ESI-HRMS and proton NMR (FIGS. 35B-35D). It is believed that the radical spin trap might increase the yield of cisplatin and subsequently result in precipitation, due to the limited solubility of cisplatin in water. Accordingly, the yield of cisplatin upon photolysis of cisPt(IV)-1 was monitored by LC-HRMS with and without DMPO or TEMPO. It is found that the presence of these radical spin traps increased the yield of cisplatin in a concentration-dependent manner (FIGS. 36, 37A-37B, and 38A-38C), and no significant difference in photolysis rates were observed following the addition of the radical spin traps (FIGS. 39 and 40A-40C). Based on these observations, it is believed that cisplatin is one of the photolysis products of cisPt(IV)-1, which is attacked by the reactive radical species generated during the photolysis process. In the presence of radical spin traps, these radical species are quenched, thereby resulting in a higher yield of cisplatin.

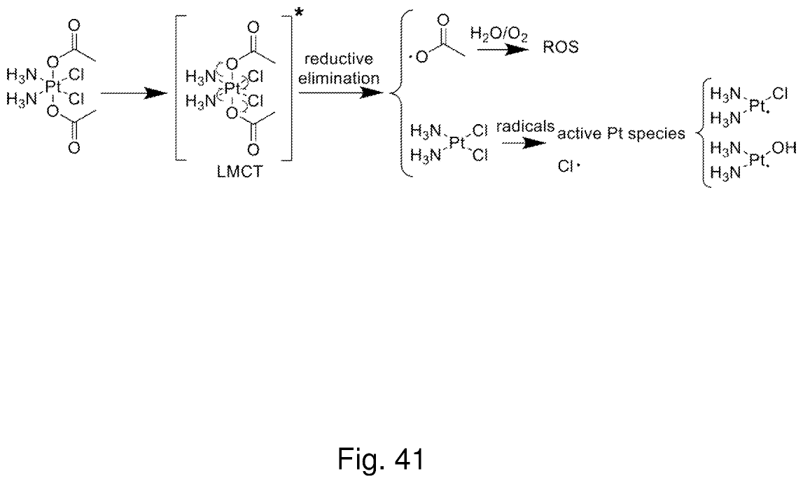

Based on the above, it is believed that the photolysis of cisPt(IV)-1 might primarily follow the mechanism as illustrated in FIG. 41. Specifically, upon irradiation, cisPt(IV)-1 is excited to the transition state via ligand-to-metal charge transfer (LMCT). Then, each acetato ligand donates one electron to the Pt(IV) center, resulting in homolytic cleavage of the Pt—O bond and subsequent reductive elimination, yielding cisplatin and acetate radicals. The latter further generates ROS in the presence of oxygen and water. These reactive radical species then attack the cisplatin, yielding various active platinum species and chlorine radicals.

### Example 5

### Photoinitiation of Polymerization and Antibacterial Properties

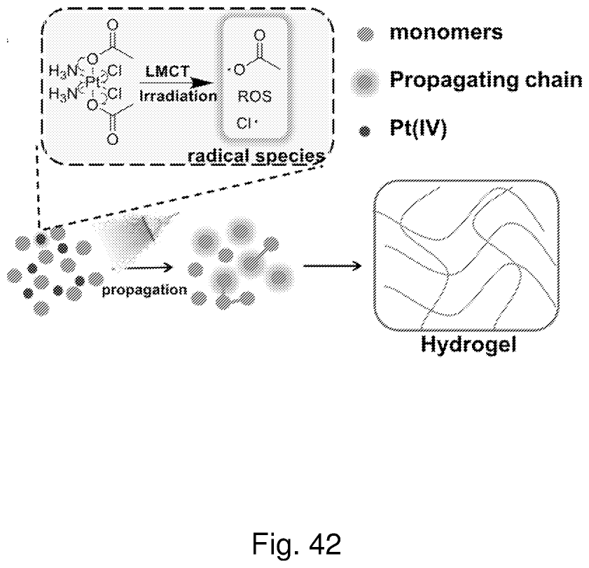

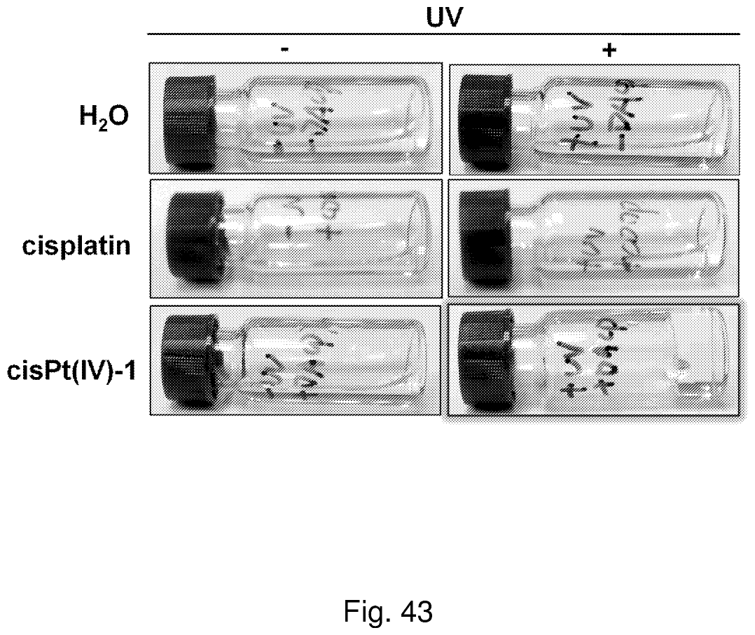

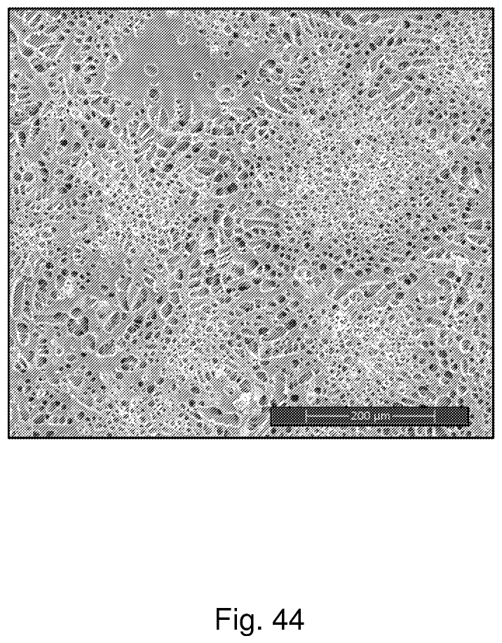

Given the generation of radical species during the photolysis of Pt(IV) complexes, it is believed that these radical species may potentially initiate the polymerization of monomers to form a macromolecular hydrogel network (FIG. 42). To examine the potential of Pt(IV) complexes as photoinitiator, a gelation experiment with acrylamide was carried out. As shown in FIG. 43, the liquid solution was transformed into a solid-like gel (Pt-gel) upon irradiation in the sample treated with cisPt(IV)-1, but not cisplatin. Scanning electron microscopy (SEM) analysis indicates that the solid-like gel has a porous network and homogeneous microstructure (FIG. 44).

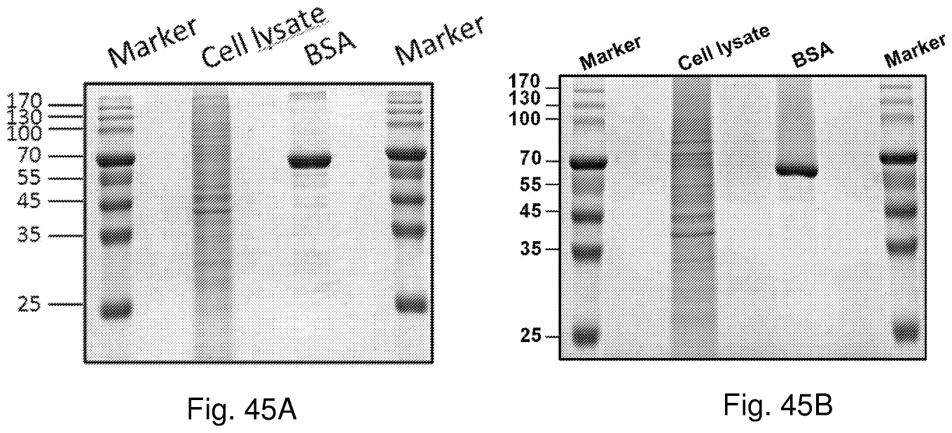

To further examine the homogeneity of this Pt-gel, gel electrophoresis of specific protein markers, cell lysate, and bovine serum albumin (BSA) was subsequently carried out using the Pt-gel. Similar to conventional APS/TEMED-gels (FIG. 45A), the protein samples separated well in the Pt-gel (FIG. 45B), suggesting the ability of Pt(IV) species to act as photoinitiators to induce photopolymerization of homogeneous polymers.

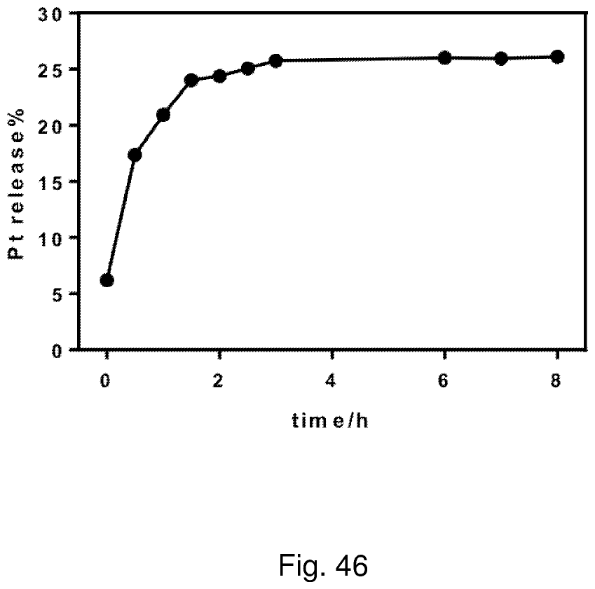

It is appreciated that low concentrations of cisplatin can inhibit cell division and induce filamentous growth in bacteria such as E. coli. Considering that the Pt(IV) complexes in this work are capable of acting as photoinitiators in the gel formation process, it is believed that, after the formation of Pt-gel, the platinum may be retained in the gel and therefore the Pt-gel may exhibit antibacterial activity. Indeed, as shown in the release profile of platinum from the Pt-gel scaffold (FIG. 46), over 75% of the photolyzed platinum species were trapped in the Pt-gel network, potentially by crosslinking.

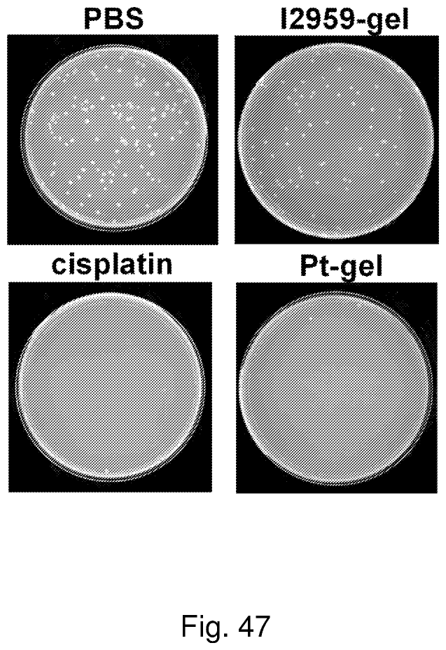

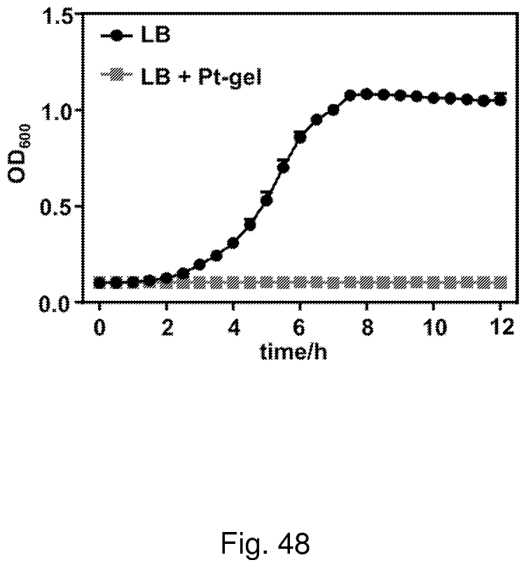

The antibacterial performance of the Pt-gel was subsequently evaluated against E. coli (DH5α), a typical pathogenic Gram-negative bacteria, using the spread plate method. Control experiments using PBS, cisplatin, and a photocrosslinked gel prepared using 12959 (a common photoinitiator) were carried out concurrently. Many colonies of bacteria could be observed on the agar plates treated with PBS and the 12959-gel. In sharp contrast, there were no visible colony-forming units (CFUs) on the agar plates treated with Pt-gel after 4 h (FIG. 47). To further confirm the antibacterial activity of the Pt-gel, E. coli was incubated with or without the Pt-gel in lysogeny broth, and the optical densities were measured at different time points. In the absence of the Pt-gel, the OD₆₀₀ values increased with incubation time and remained stable after 8 h, consistent with the growth pattern of normal bacteria. In contrast, the OD₆₀₀ values remained low and unchanged in the Pt-gel-treated sample, suggesting excellent antibacterial property of the Pt-gel (FIG. 48).

### Example 6

### Photocrosslinking Properties

In considering the photoinduced reactive Pt species, including the platinum radicals and Pt(II) species generated from the Pt(IV) complexes in this work, which could potentially react with various biological nucleophiles (i.e., the Pt(IV) complexes may be considered to be photocaged electrophiles), it is believed that the Pt(IV) complexes may be used as photocrosslinker and/or photoreactive biomolecule-labeling reagents.

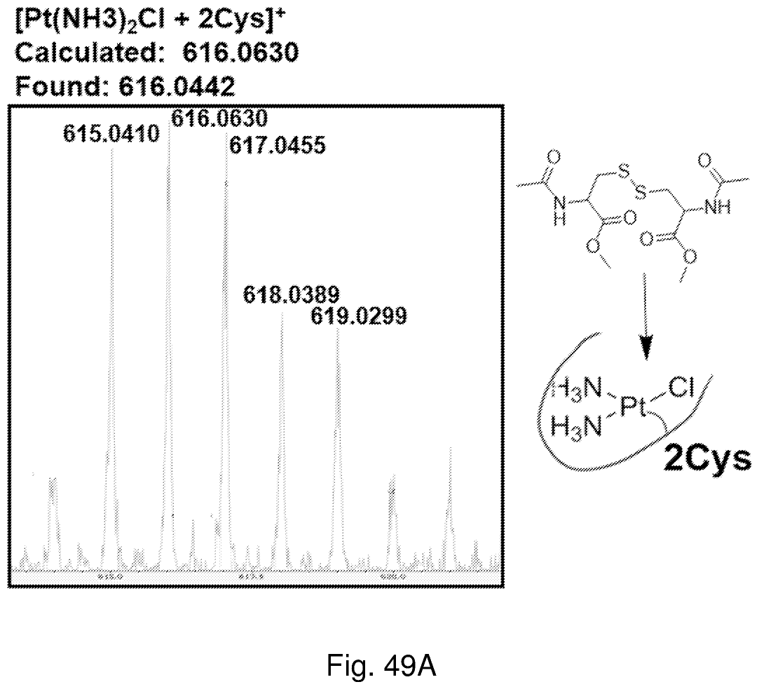

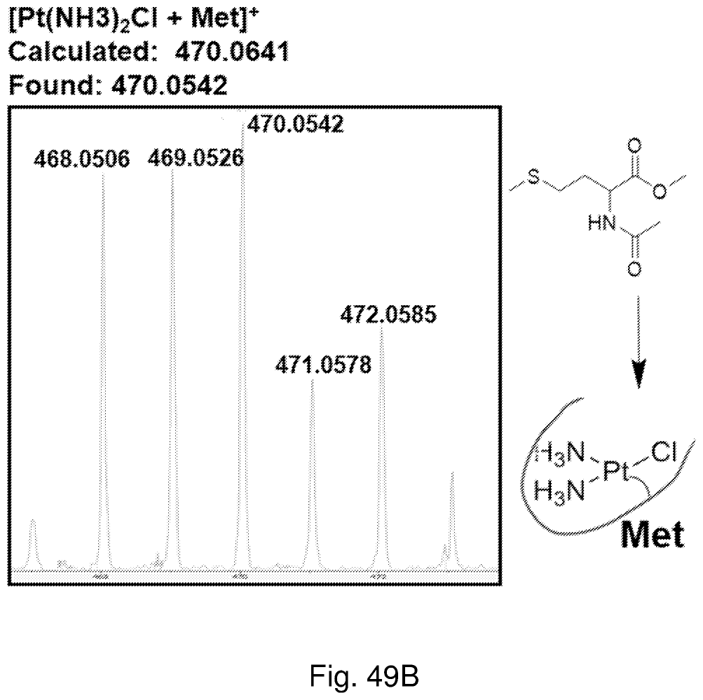

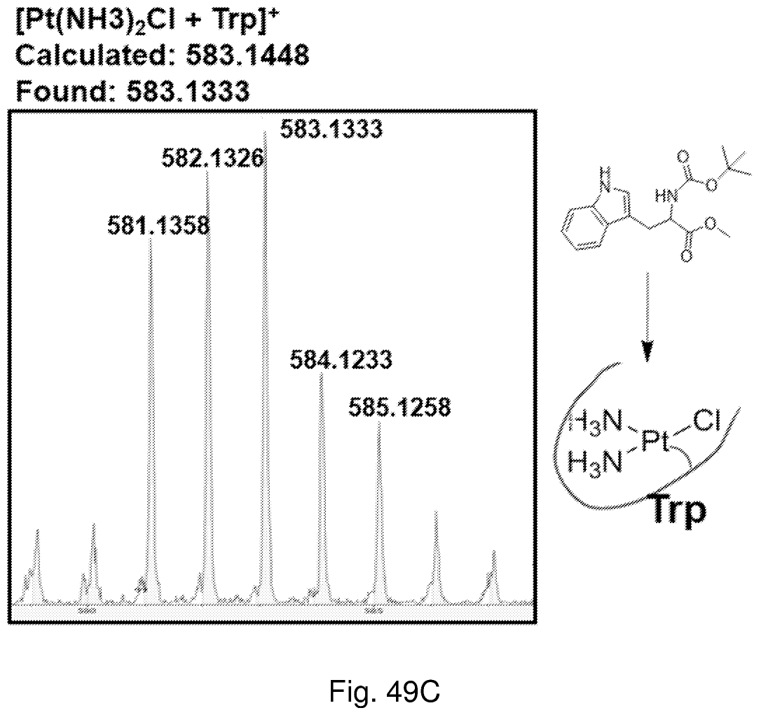

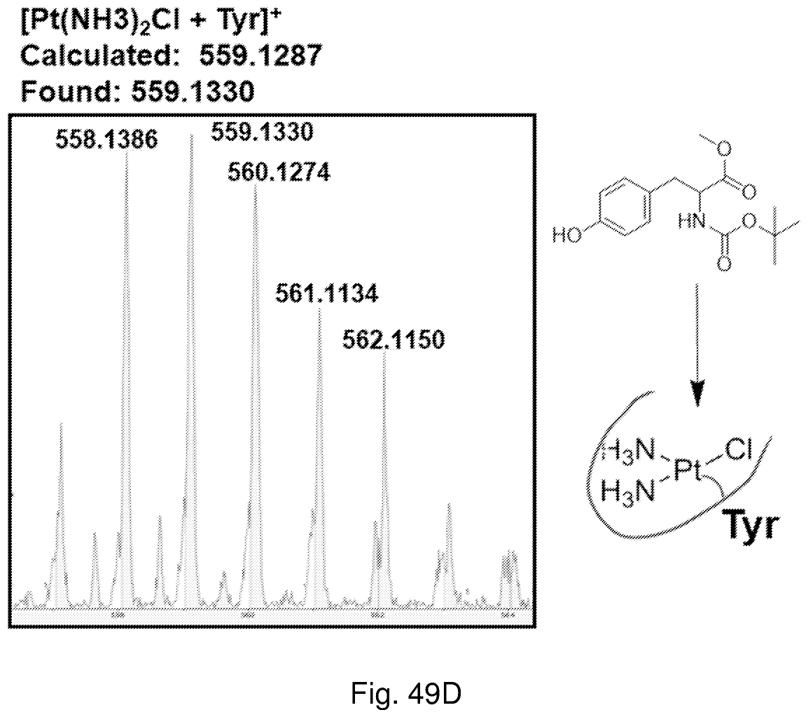

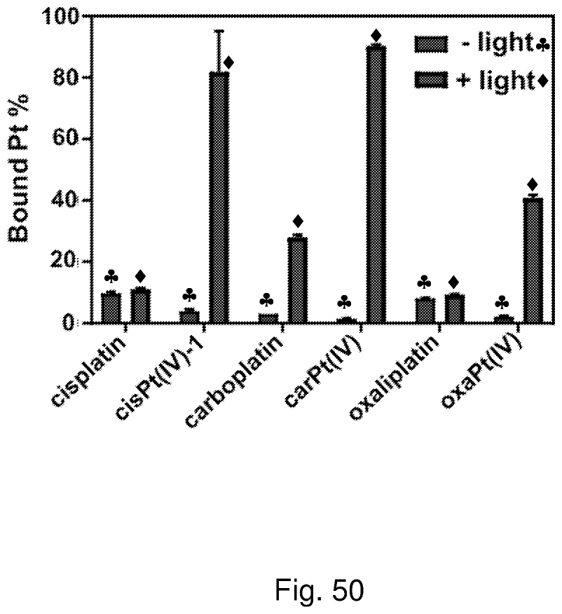

The reactivity of the Pt(IV) complexes with individual model amino acids upon photolysis was investigated. As shown in FIGS. 49A-49D, Pt-amino acid adducts were observed for all tested amino acids. Then, the binding efficiency of the Pt(IV) complexes with bovine serum albumin (BSA) was examined using ICP-OES. Without irradiation, both Pt(II) and Pt(IV) species displayed a limited ability to bind protein; less than 10% of platinum complexes bound to the BSA, and the Pt(IV) complexes exhibited a slightly lower binding ratio than the Pt(II) counterparts due to their greater kinetic inertness. In contrast, following irradiation, a significant increase in binding ratio was observed in the Pt(IV)-treated samples. In particular, cisPt(IV)-1 and carPt(IV) showed over 80% of the platinum complexes bound to BSA, indicating that the photolyzed platinum products could (photo) crosslink to the protein efficiently (FIG. 50).

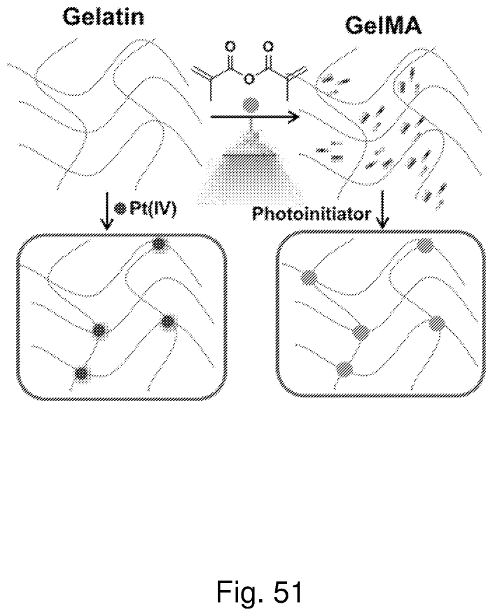

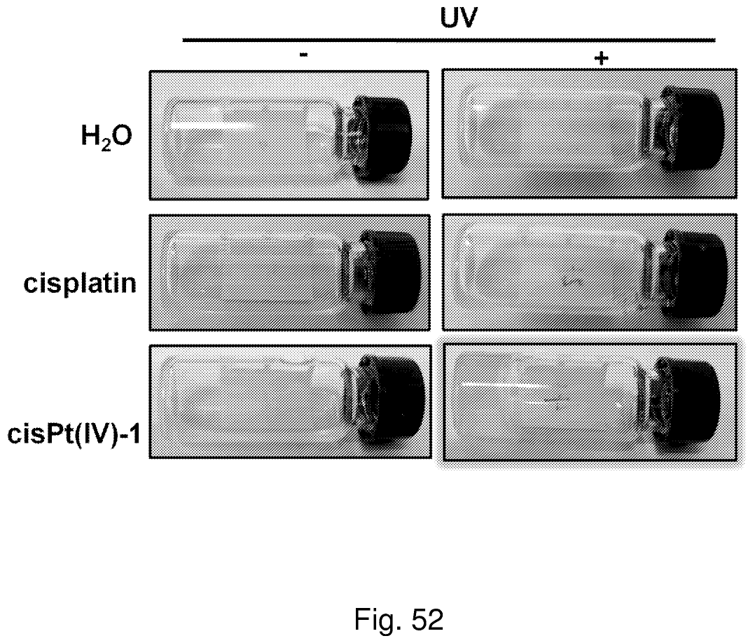

Given the above efficient protein photocrosslinking properties of the Pt(IV) complexes, it is believed that the Pt(IV) species may act as photocrosslinkers to directly produce a gelatin hydrogel (FIG. 51). To confirm the Pt(IV) complexes could achieve as such, gelation experiments using cisplatin and cisPt(IV)-1 were performed. As shown in FIG. 52, the formation of gelatin hydrogel could be clearly observed in the cisPt(IV)-1-treated samples upon irradiation, but not the cisplatin-treated samples, suggesting that the Pt(IV) complexes can be used as photocrosslinkers for the direct fabrication of gelatin hydrogel scaffolds.

### Example 7

### Photolabeling Properties

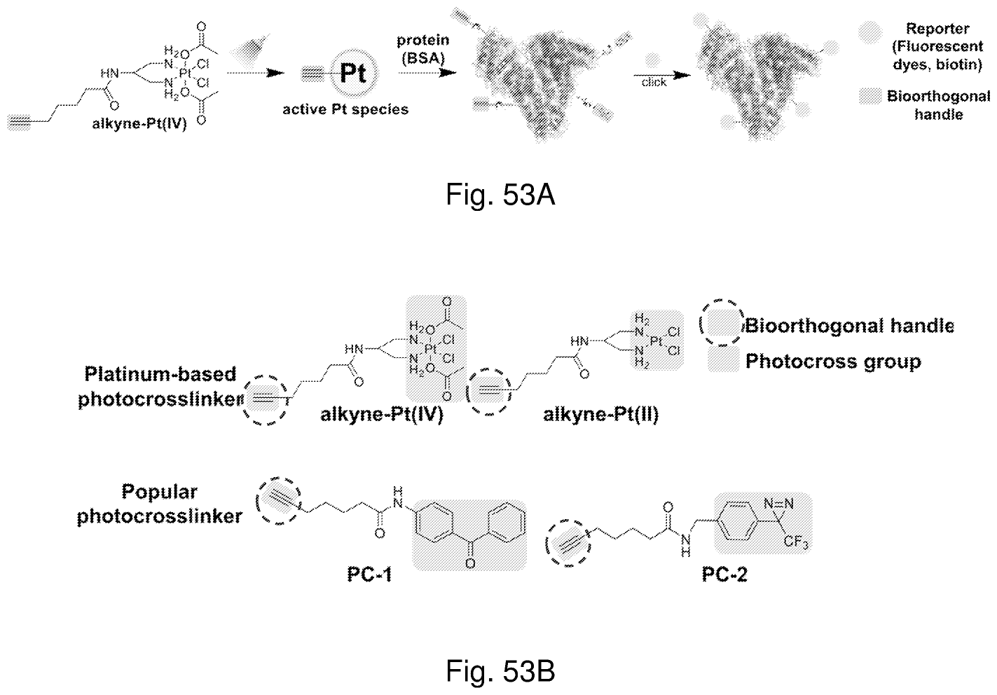

To further investigate the photocrosslinking and photolabeling properties of the Pt(IV) compelxes of the present invention, a novel photocrosslinker alkyne-Pt(IV), which is based on cisPt(IV)-1 and contains a biorthogonal handle alkyne, was designed and synthesized (FIGS. 7A-7E and 53A-53B). Upon photolysis, the reactive platinum species could crosslink to proteins instantly, allowing the subsequent introduction of reporters, such as fluorescence groups, via click chemistry (FIG. 53A). The labeling efficiency of alkyne-Pt(IV) was subsequently evaluated using BSA. Common benzophenol- and diazirine-based photocrosslinkers, PC-1 and PC-2, were synthesized for comparison (FIGS. 8A-8D, 9A-9D, and 53B). A Pt(II) counterpart, alkyne-Pt(II), was also synthesized as a control (FIGS. 53B and 10A-10D).

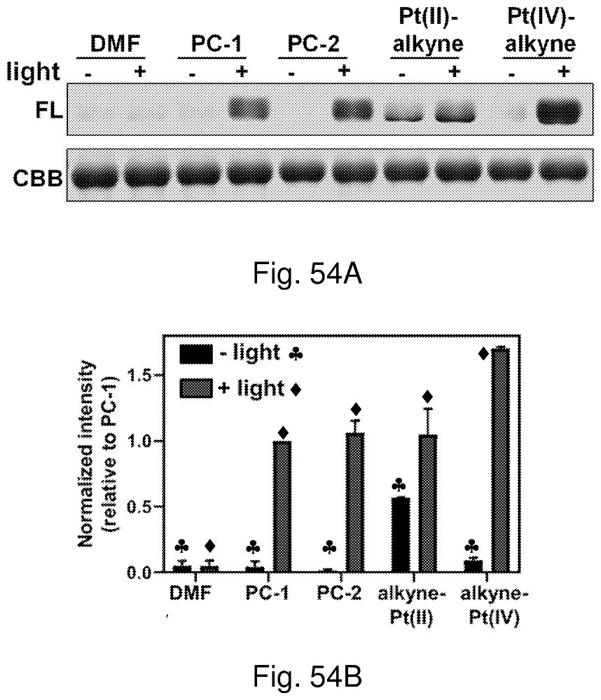

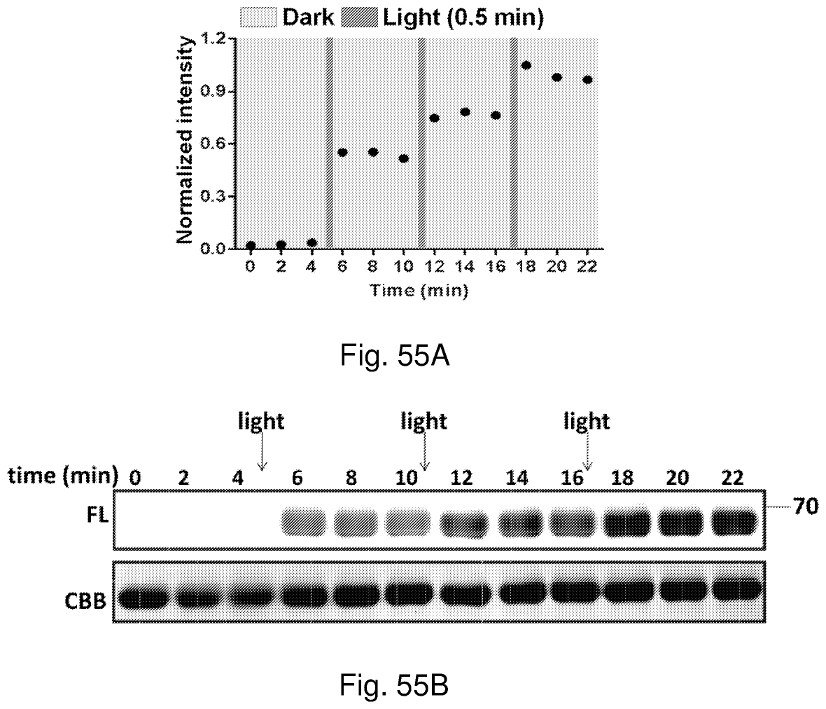

Without irradiation, all probes exhibited low labeling efficiency, with alkyne-Pt(II) exhibited a higher labeling efficiency than alkyne-Pt(IV). In sharp contrast, under irradiation, a significant enhancement in fluorescence was observed in the sample treated with alkyne-Pt(IV). The fluorescence intensity from alkyne-Pt(IV) was 1.7-fold higher than those of the organic probes PC-1 and PC-2 (FIGS. 54A and 54B). Furthermore, to examine the kinetics of the photocrosslinking process, pulse-chase labeling of proteins was carried out by switching the light on and off. The light dependence of the protein labeling was observed, with distinct increases in the fluorescence intensity following each pulse of light, indicating the instant nature of the crosslinking process and the temporal specificity afforded by the use of external light (FIGS. 55A and 55B).

The invention has been given by way of example only, and various other modifications of and/or alterations to the described embodiment may be made by persons skilled in the art without departing from the scope of the invention as specified in the appended claims.

1. A platinum-based adduct comprising a substrate to which a photolyzed active platinum species is attached, the photolyzed active platinum species is generated from a platinum(IV) complex having a structure of Formula (Ia): 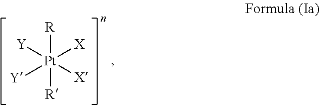
wherein X, X′, Y, Y′, R and R′ are electron donor ligands; and n is zero, any positive charge or negative charge.

2. The platinum-based adduct as claimed in claim 1, wherein at least one of the electron donor ligand comprises one or more of nitrogen-containing ligands, oxygen-containing ligands, phosphorous-containing ligands, sulfur-containing ligands or halogen-containing ligands.

3. The platinum based adduct as claimed in claim 1, wherein the photolyzed active platinum species comprises a platinum(I) radical.

4. The platinum-based adduct as claimed in claim 1, wherein the platinum(IV) complex has a structure of Formula (Ib): 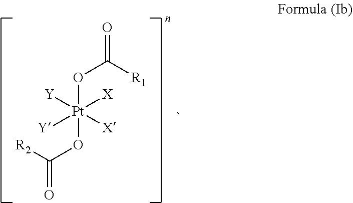
wherein:
X, X′, Y, and Y′ are independently selected from the group consisting of ammonia, halide, oxalate, diamines, dicarboxylate, and glycolate,
R₁ and R₂ are independently selected from the group consisting of a linear or branched C1-C4 alkyl chain, a carboxyl group, and a carboxylate ester-containing group; and
n is zero charge.

5. The platinum-based adduct as claimed in claim 4, wherein X and X′ are linked to form a first bidentate ligand.

6. The platinum-based adduct as claimed in claim 4, wherein Y and Y′ are linked to form a second bidentate ligand.

7. The platinum-based adduct as claimed in claim 1, wherein the substrate is hydrogel-based or biomolecule-based.

8. The platinum-based adduct as claimed in claim 4, wherein R₁ and R₂ are independently selected from the group consisting of —CH₃, —CH₂CH₃, —CH₂CH₂CH₃, —CH₂COOH, —CH₂CH₂COOH, —CH₂COOCH₃, —CH₂CH₂COOCH₃ and —CH₂COOCH₂CH₃.

9. The platinum-based adduct as claimed in claim 6, wherein X and X′ are ammonia and/or chloride; the second bidentate ligand is a diamine; R₁ and R₂ are —CH₃, —CH₂CH₂COOH, or —CH₂CH₂COOCH₃; and n is zero charge.

10. The platinum-based adduct as claimed in claim 5, wherein Y and Y′ are ammonia and/or chloride, the first bidentate ligand is cyclobutane dicarboxylate and/or oxalate, R₁ and R₂ are —CH₃, —CH₂CH₂COOH or —CH₂CH₂COOCH₃; and n is zero charge.

11. The platinum-based adduct as claimed in claim 4, wherein the platinum(IV) complex has a structure of Formula (IIa), Formula (IIb), Formula (IIc), Formula (IId), Formula (IIe), or Formula (IIf):

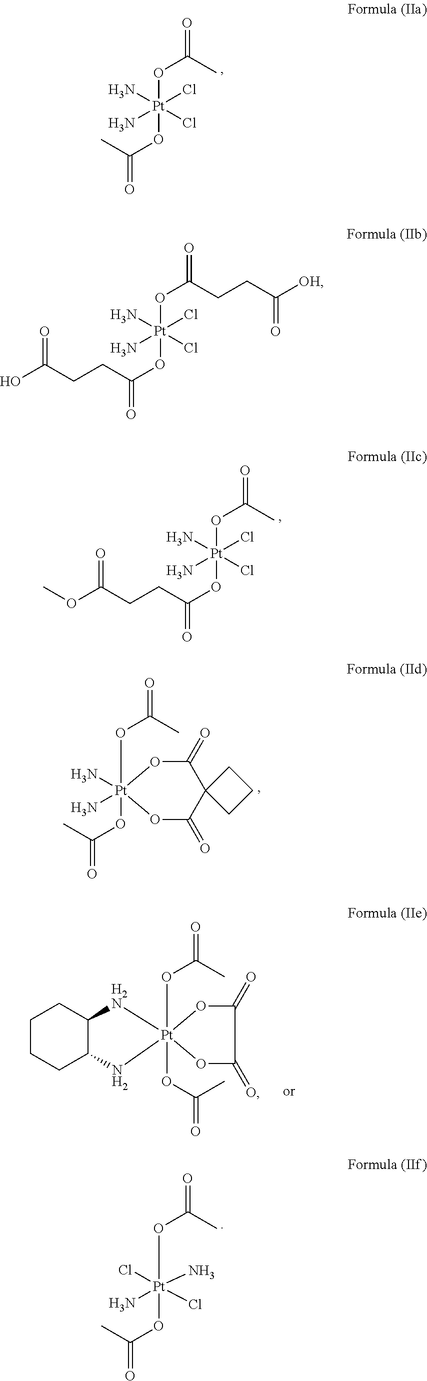

12. The platinum-based adduct as claimed in claim 11, wherein the platinum(IV) complex of Formula (IIa) generates one or more of acetate radical, cisplatin, chlorine radical, ROS and the photolyzed active platinum species.

13. The platinum-based adduct as claimed in claim 11, wherein the chemical structure of the platinum(IV) complex remains unchanged for at least 30 min in the absence of radiation.

14. The platinum-based adduct as claimed in claim 7, wherein the hydrogel-based substrate comprises any one of polyacrylamide or gelatin.

15. The platinum-based adduct as claimed in claim 7, wherein the biomolecule-based substrate comprises any one of an amino acid or polypeptide.

16. The platinum-based adduct as claimed in claim 14, wherein the hydrogel-based substrate comprises any one of 10% acrylamide/bis-acrylamide (29:1) and 5% gelatin.

17. The platinum-based adduct as claimed in claim 15, wherein the amino acid is selected from cysteine, methionine, tryptophan, and tyrosine, and the polypeptide is BSA.

18. The platinum-based adduct as claimed in claim 4, wherein the platinum(IV) complex further comprises a functional moiety that is attached to the platinum(IV) complex for functionalizing the platinum-based adduct.

19. The platinum-based adduct as claimed in claim 18, wherein the platinum(IV) complex has a structure of Formula (IIIa): 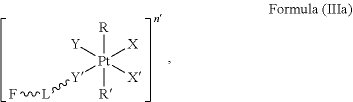
wherein:
X, X′, Y, Y′, R and R′ are as defined above;
L is a linker group;
F is the functional moiety for functionalizing the platinum-based adduct; and
n′ is zero, any positive charge or negative charge.

20. The platinum-based adduct as claimed in claim 19, wherein the functional moiety has a reactive group that is complementary to the reactive group of a fluorescent reporting unit.

21. The platinum-based adduct as claimed in claim 20, wherein the platinum(IV) complex has a structure of Formula (IIIb): 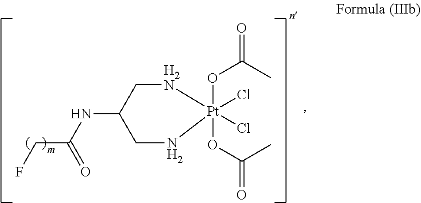
wherein:
m is 1-6;
F is the functional moiety which is a biorthogonal group selected from the group consisting of azide, terminal alkyne, activated cyclooctyne, and tetrazine; and
n′ is zero charge.

22. The platinum-based adduct as claimed in claim 21, wherein the platinum(IV) has a structure of Formula (IV):

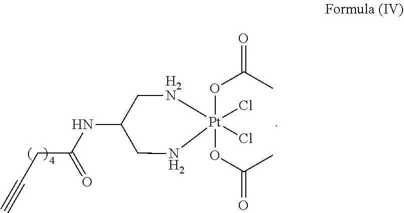

23. A photoinitiator for hydrogelation comprising a photolyzed active platinum species generated from a platinum(IV) complex having a structure of Formula (Ia): 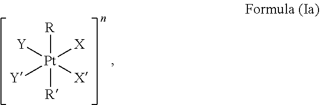
wherein X, X′, Y, Y′, R and R′ are electron donor ligands; and n is zero, any positive charge or negative charge.

24. The photoinitiator as claimed in claim 23, wherein the platinum(IV) complex has a structure of Formula (Ib): 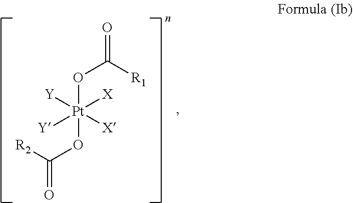
wherein:
X, X′, Y, and Y′ are independently selected from the group consisting of ammonia, halide, oxalate, diamines, dicarboxylate, and glycolate,
R₁ and R₂ are independently selected from the group consisting of a linear or branched C1-C4 alkyl chain, a carboxyl group, and a carboxylate ester-containing group; and
n is zero charge.

25. The photoinitiator as claimed in claim 23, wherein 75% of photolyzed platinum species is retained after exposure to radiation.

26. The photoinitiator as claimed in claim 24, wherein the platinum(IV) complex has a structure of Formula (IIa):

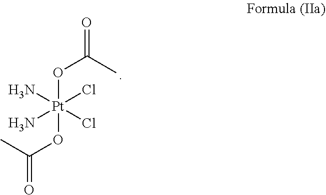

27. A photocrosslinker for hydrogelation or protein labeling comprising a photolyzed active platinum species generated from a platinum(IV) complex having a structure of Formula (Ia) or Formula (IIIa): 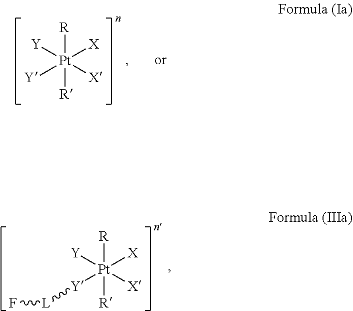
wherein:
X, X′, Y, Y′, R and R′ are electron donor ligands;
L is a linker group;
F is a functional moiety for protein labeling; and
n and n′ are each independently being zero, any positive charge or negative charge.

28. The photocrosslinker as claimed in claim 27, wherein the platinum(IV) complex has a structure of Formula (Ib): 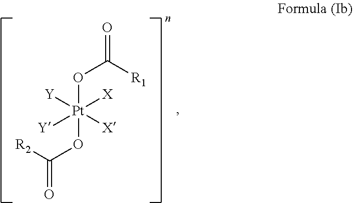
wherein:
X, X′, Y, and Y′ are independently selected from the group consisting of ammonia, halide, oxalate, diamines, dicarboxylate, and glycolate,
R₁ and R₂ are independently selected from the group consisting of a linear or branched C1-C4 alkyl chain, a carboxyl group, and a carboxylate ester-containing group; and
n is zero charge.

29. The photocrosslinker as claimed in claim 28, wherein the platinum(IV) complex has a structure of Formula (IIa):

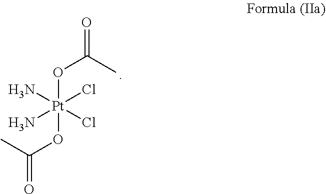

30. The photocrosslinker as claimed in claim 27, wherein the platinum(IV) complex has a structure of Formula (IIIb): 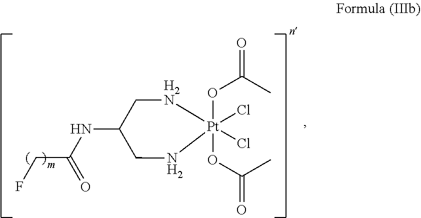
wherein:
m is 1-6;
F is the functional moiety which is a biorthogonal group selected from the group consisting of azide, terminal alkyne, activated cyclooctyne, and tetrazine; and
n′ is zero charge.

31. The photocrosslinker as claimed in claim 30, wherein the platinum(IV) has a structure of Formula (IV):

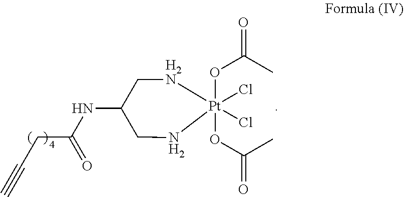

32. An antibacterial platinum-based adduct comprising a substrate to which a photolyzed active platinum species is attached, the photolyzed active platinum species is generated from a platinum(IV) complex having a structure of Formula (IIa):

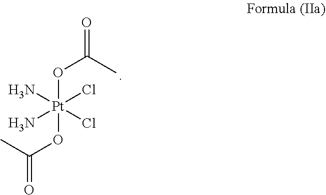

33. The antibacterial platinum-based adduct as claimed in claim 32, wherein the substrate comprises polyacrylamide and has an antibacterial activity against E. coli (DH5α).

34. A method of preparing the platinum-based adduct as claimed in claim 1, comprising the steps of:
providing a solution mixture comprising a hydrogel precursor or a biomolecule, and a platinum(IV) complex having a structure of Formula (Ia); and
administering to the solution mixture radiation in an amount and of a wavelength effective to activate the platinum(IV) complex to form a photolyzed platinum species comprising a platinum(1) radical, which reacts with the hydrogel precursor or the biomolecule to form the platinum-based adduct.

35. The method as claimed in claim 34, wherein the radiation is UV radiation.

36. The method as claimed in claim 35, wherein the UV radiation has a wavelength of about 360 nm to about 370 nm.

37. The method as claimed in claim 36, wherein the UV radiation is applied at a power density from about 9 mW/cm² to about 10 mW/cm².
